(19) United States
(12) Patent Application Publication
(10) Pub. No.: US 2023/0183193 A1
(43) Pub. Date: Jun. 15, 2023

(54) 4-DIFLUOROMETHYL BENZOYL AMIDES WITH HERBICIDAL ACTION

(71) Applicant: BAYER CROPSCIENCE AKTIENGESELLSCHAFT, Monheim am Rhein (DE)

(72) Inventors: Hartmut AHRENS, Langen (DE); Joerg TIEBES, Frankfurt (DE); Christian WALDRAFF, Bad Vilbel (DE); Hansjoerg DIETRICH, Liederbach am Taunus (DE); Elmar GATZWEILER, Bad Nauheim (DE); Christopher Hugh ROSINGER, Hofheim (DE); Anu Bheemaiah MACHETTIRA, Frankfurt am Main (DE)

(21) Appl. No.: 18/054,089

(22) Filed: Nov. 9, 2022

Related U.S. Application Data
(63) Continuation of application No. 16/610,216, filed on Nov. 1, 2019, now abandoned.

(30) Foreign Application Priority Data
May 4, 2017 (EP) 17169505.9

(51) Int. Cl. C07D 257/04; A01N 43/26; A01N 43/653; A01N 43/82; C07C 317/14; C07C 321/28; C07D 255/02; C07D 271/04; C07D 271/06

(52) U.S. Cl. CPC C07D 257/04; A01N 43/26; A01N 43/653; A01N 43/82; C07C 317/14; C07C 321/28; C07D 255/02; C07D 271/04; C07D 271/06

(57) ABSTRACT

Benzoylamides of the general formula (I) are described as herbicides.

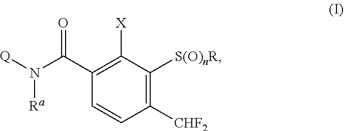

In this formula (I), X, R and R^{a} represent radicals such as alkyl, cycloalkyl and halogen. Q represents a five-membered heterocycle.

### CROSS-REFERENCE TO RELATED APPLICATIONS

This application is a continuation of U.S. application Ser. No. 16/610,216, filed 1 Nov. 2019, which is a continuation of a National Stage entry of International Application No. PCT/EP2018/060709, filed 26 Apr. 2018, which claims priority to European Patent Application No. 17169505.9, filed 4 May 2017. The content of each of these applications is herein incorporated by reference in their entirety.

### BACKGROUND

### Field

The invention relates to the technical field of the herbicides, especially that of the herbicides for selective control of weeds and weed grasses in crops of useful plants.

### Description of the Related Art

WO 2011/035874 A1, WO 2012/126932 A1, WO 2012/028579 A1 and WO 2016/146561 A1 describe herbicidally active benzoylamides which differ from one another essentially by the nature of the heterocyclic substituent. These benzoylamides may be substituted in the 2-, 3- and 4-positions of the phenyl ring by a large number of different radicals. WO 2016/146561 A1 discloses, under the tabulated examples 1-38 and 1-41, the sodium salts of the two compounds 4-difluoromethyl-3-ethylsulfinyl-2-methyl-N-(5-methyl-1,3,4-oxadiazol-2-yl)benzamide and 4-difluoromethyl-3-ethylsulfonyl-2-methyl-N-(5-methyl-1,3,4-oxadiazol-2-yl)benzamide. However, the benzoylamides known from the publications mentioned above do not always have adequate herbicidal efficacy and/or compatibility with crop plants.

### SUMMARY

It is an object of the present invention to provide alternative herbicidally active compounds. This object is achieved by the benzoylamides according to the invention described below, which carry an alkyl, cycloalkyl or halogen group in the 2-position of the phenyl ring, a sulfur-containing radical in the 3-position and a CHF₂ group in the 4-position.

Accordingly, the present invention provides benzoylamides of the formula (I) and salts thereof

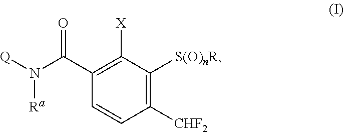

in which the symbols and indices are defined as follows:

Q represents a radical Q1, Q2, Q3 or Q4,

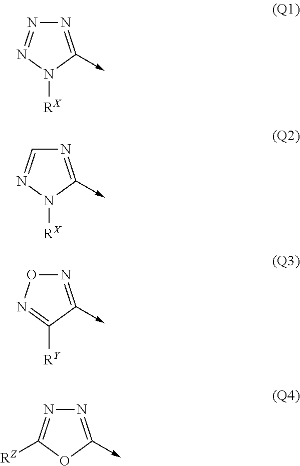

X represents (C₁-C₆)-alkyl, (C₃-C₆)-cycloalkyl or halogen,

R represents (C₁-C₆)-alkyl, (C₃-C₆)-cycloalkyl, (C₃-C₆)-cycloalkyl-(C₁-C₆)-alkyl, (C₁-C₆)-alkyl-O—(C₁-C₆)-alkyl,

R^{a} represents hydrogen, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₆)-cycloalkyl, halo-(C₃-C₆)-cycloalkyl, (C₃-C₆)-cycloalkyl-(C₁-C₆)-alkyl, halo-(C₃-C₆)-cycloalkyl-(C₁-C₆)-alkyl, R¹(O)C—(C₁-C₆)-alkyl, R¹O(O)C—(C₁-C₆)-alkyl, (R¹)₂N(O)C—(C₁-C₆)-alkyl, NC—(C₁-C₆)-alkyl, R¹O—(C₁-C₆)-alkyl, R¹(O)CO—(C₁-C₆)-alkyl, R²(O)₂SO—(C₁-C₆)-alkyl, (R¹)₂N—(C₁-C₆)-alkyl, R¹(O)C(R¹)N—(C₁-C₆)-alkyl, R²(O)₂S(R¹)N—(C₁-C₆)-alkyl, R²(O)ₙS—(C₁-C₆)-alkyl, R¹O(O)₂S—(C₁-C₆)-alkyl, (R¹)₂N(O)₂S—(C₁-C₆)-alkyl, R¹(O)C, R¹O(O)C, (R¹)₂N(O)C, R¹O, (R¹)₂N, R²O(O)C(R¹)N, (R¹)₂N(O)C(R¹)N, R²(O)₂S, or benzyl substituted in each case by s radicals from the group consisting of methyl, ethyl, methoxy, nitro, trifluoromethyl and halogen,

R^{X} represents (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, where the six radicals mentioned above are in each case substituted by s radicals from the group consisting of nitro, cyano, (R⁶)₃Si, (R⁵O)₂(O)P, R²(O)ₙS, (R¹)₂N, R¹O, R¹(O)C, R¹O(O)C, R¹(O)CO, R²O(O)CO, R¹(O)C(R¹)N, R²(O)₂S(R¹)N, (C₃-C₆)-cycloalkyl, heteroaryl, heterocyclyl and phenyl, where the four last-mentioned radicals are substituted by s radicals from the group consisting of (C₁-C₆)-alkyl, halo-(C₁-C₆)-alkyl, (C₁-C₆)-alkoxy, halo-(C₁-C₆)-alkoxy and halogen, and where heterocyclyl carries n oxo groups,

or R^{X} represents (C₃-C₇)-cycloalkyl, heteroaryl, heterocyclyl or phenyl, where the four radicals mentioned above are in each case substituted by s radicals from the group consisting of halogen, nitro, cyano, (C₁-C₆)-alkyl, halo-(C₁-C₆)-alkyl, (C₃-C₆)-cycloalkyl, (C₁-C₆)-alkyl-S(O)ₙ, (C₁-C₆)-alkoxy, halo-(C₁-C₆)-alkoxy and (C₁-C₆)-alkoxy-(C₁-C₄)-alkyl,

R^{Y} represents hydrogen, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₇)-cycloalkyl, (C₁-C₆)-alkoxy, halo-(C₁-C₆)-alkoxy, (C₂-C₆)-alkenyloxy, (C₂-C₆)-alkynyloxy, cyano, nitro, methylsulfenyl, methylsulfinyl, methylsulfonyl, acetylamino, benzoylamino, methoxycarbonyl, ethoxycarbonyl, methoxycarbonylmethyl, ethoxycarbonylmethyl, benzoyl, methylcarbonyl, piperidinylcarbonyl, trifluoromethylcarbonyl, halogen, amino, aminocarbonyl, methylaminocarbonyl, dimethylaminocarbonyl, methoxymethyl, or represents heteroaryl, heterocyclyl or phenyl, each of which is substituted by s radicals from the group consisting of (C₁-C₆)-alkyl, halo-(C₁-C₆)-alkyl, (C₁-C₆)-alkoxy, halo-(C₁-C₆)-alkoxy and halogen, and where heterocyclyl carries n oxo groups,

R^{Z} represents hydrogen, (C₁-C₆)-alkyl, R¹O—(C₁-C₆)-alkyl, R′₂CH₂, (C₃-C₇)-cycloalkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, R¹O, R¹(H)N, methoxycarbonyl, ethoxycarbonyl, methylcarbonyl, dimethylamino, trifluoromethylcarbonyl, acetylamino, methylsulfenyl, methylsulfinyl, methylsulfonyl, or represents heteroaryl, heterocyclyl, benzyl or phenyl, each of which is substituted by s radicals from the group consisting of halogen, nitro, cyano, (C₁-C₆)-alkyl, halo-(C₁-C₆)-alkyl, (C₃-C₆)-cycloalkyl, (C₁-C₆)-alkyl-S(O)ₙ, (C₁-C₆)-alkoxy, halo-(C₁-C₆)-alkoxy and (C₁-C₆)-alkoxy-(C₁-C₄)-alkyl, where heterocyclyl carries n oxo groups,

R¹ represents hydrogen, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₆)-cycloalkyl, (C₃-C₆)-cycloalkenyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, phenyl-N(R³)—(C₁-C₆)-alkyl, heteroaryl-N(R³)—(C₁-C₆)-alkyl, heterocyclyl-N(R³)—(C₁-C₆)-alkyl, phenyl-S(O)ₙ—(C₁-C₆)-alkyl, heteroaryl-S(O)ₙ—(C₁-C₆)-alkyl or heterocyclyl-S(O)ₙ—(C₁-C₆)-alkyl, where the fifteen last-mentioned radicals are in each case substituted by s radicals from the group consisting of nitro, halogen, cyano, thiocyanato, (C₁-C₆)-alkyl, halo-(C₁-C₆)-alkyl, (C₃-C₆)-cycloalkyl, R³O(O)C, (R³)₂N(O)C, R³O, (R³)₂N, R⁴(O)ₙS, R³O(O)₂S, (R³)₂N(O)₂S and R³O—(C₁-C₆)-alkyl, and where heterocyclyl carries n oxo groups,

R² represents (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₆)-cycloalkyl, (C₃-C₆)-cycloalkenyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, phenyl-N(R³)—(C₁-C₆)-alkyl, heteroaryl-N(R³)—(C₁-C₆)-alkyl, heterocyclyl-N(R³)—(C₁-C₆)-alkyl, phenyl-S(O)ₙ—(C₁-C₆)-alkyl, heteroaryl-S(O)ₙ—(C₁-C₆)-alkyl or heterocyclyl-S(O)ₙ—(C₁-C₆)-alkyl, where the fifteen last-mentioned radicals are in each case substituted by s radicals from the group consisting of nitro, halogen, cyano, thiocyanato, (C₁-C₆)-alkyl, halo-(C₁-C₆)-alkyl, (C₃-C₆)-cycloalkyl, R³O(O)C, (R³)₂N(O)C, R³O, (R³)₂N, R⁴(O)ₙS, R³O(O)₂S, (R³)₂N(O)₂S and R³O—(C₁-C₆)-alkyl, and where heterocyclyl carries n oxo groups,

R³ represents hydrogen, (C₁-C₆)-alkyl, halo-(C₁-C₆)-alkyl, (C₂-C₆)-alkenyl, (C₂-C₆)-alkynyl, (C₃-C₆)-cycloalkyl, (C₃-C₆)-cycloalkyl-(C₁-C₆)-alkyl or phenyl,

R⁴ represents (C₁-C₆)-alkyl, halo-(C₁-C₆)-alkyl, (C₂-C₆)-alkenyl, (C₂-C₆)-alkynyl, (C₃-C₆)-cycloalkyl, (C₃-C₆)-cycloalkyl-(C₁-C₆)-alkyl or phenyl,

R⁵ represents hydrogen or (C₁-C₄)-alkyl,

R⁶ represents (C₁-C₄)-alkyl,

R′ represents acetoxy, acetamido, N-methylacetamido, benzoyloxy, benzamido, N-methylbenzamido, methoxycarbonyl, ethoxycarbonyl, benzoyl, methylcarbonyl, piperidinylcarbonyl, morpholinylcarbonyl, trifluoromethylcarbonyl, aminocarbonyl, methylaminocarbonyl, dimethylaminocarbonyl, (C₃-C₆)-cycloalkyl, or represents heteroaryl or heterocyclyl, in each case substituted by s radicals from the group consisting of methyl, ethyl, methoxy, trifluoromethyl and halogen,

n represents 0, 1 or 2,

s represents 0, 1, 2 or 3,

with the proviso that the compounds 4-difluoromethyl-3-ethylsulfinyl-2-methyl-N-(5-methyl-1,3,4-oxadiazol-2-yl)benzamide and 4-difluoromethyl-3-ethylsulfonyl-2-methyl-N-(5-methyl-1,3,4-oxadiazol-2-yl)benzamide and their sodium salts are excluded.

In the radicals Q1, Q2, Q3 and Q4, the arrow denotes the bond to the amide nitrogen atom of the compounds of the formula (I).

### DETAILED DESCRIPTION OF A PREFERRED EMBODIMENT

In the formula (I) and all the formulae which follow, alkyl radicals having more than two carbon atoms may be straight-chain or branched. Alkyl radicals are, for example, methyl, ethyl, n-propyl or isopropyl, n-, iso-, t- or 2-butyl, pentyls, hexyls such as n-hexyl, isohexyl and 1,3-dimethylbutyl. Analogously, alkenyl is, for example, allyl, 1-methylprop-2-en-1-yl, 2-methylprop-2-en-1-yl, but-2-en-1-yl, but-3-en-1-yl, 1-methylbut-3-en-1-yl and 1-methylbut-2-en-1-yl. Alkynyl is, for example, propargyl, but-2-yn yl, but-3-yn-1-yl, 1-methylbut-3-yn-1-yl. The multiple bond may be in any position in each unsaturated radical. Cycloalkyl is a carbocyclic saturated ring system having three to six carbon atoms, for example cyclopropyl, cyclobutyl, cyclopentyl or cyclohexyl.

Halogen is fluorine, chlorine, bromine or iodine.

Depending on the nature of the substituents and the manner in which they are attached, the compounds of the general formula (I) may be present as stereoisomers. If, for example, one or more asymmetrically substituted carbon atoms are present, there may be enantiomers and diastereomers. Stereoisomers likewise occur when n is 1 (sulfoxides). Stereoisomers can be obtained from the mixtures obtained in the preparation by customary separation methods, for example by chromatographic separation processes. It is likewise possible to selectively prepare stereoisomers by using stereoselective reactions with use of optically active starting materials and/or auxiliaries. The invention also relates to all the stereoisomers and mixtures thereof that are encompassed by the general formula (I) but are not defined specifically.

The compounds of the formula (I) are capable of forming salts. Suitable bases are, for example, organic amines such as trialkylamines, morpholine, piperidine or pyridine, and the hydroxides, carbonates and bicarbonates of ammonium, alkali metals or alkaline earth metals, especially sodium hydroxide, potassium hydroxide, sodium carbonate, potassium carbonate, sodium bicarbonate and potassium bicarbonate. These salts are compounds in which the acidic hydrogen is replaced by an agriculturally suitable cation, for example metal salts, especially alkali metal salts or alkaline earth metal salts, in particular sodium and potassium salts, or else ammonium salts, salts with organic amines or quaternary ammonium salts, for example with cations of the formula [NRR′R″R′″]⁺ in which R to R′″ each independently of one another represent an organic radical, in particular alkyl, aryl, aralkyl or alkylaryl. Also suitable are alkylsulfonium and alkylsulfoxonium salts, such as (C₁-C₄)-trialkylsulfonium and (C₁-C₄)-trialkylsulfoxonium salts.

The compounds of the formula (I) can form salts through adduct formation of a suitable inorganic or organic acid, for example mineral acids such as HCl, HBr, H₂SO₄, H₃PO₄ or HNO₃, or organic acids, for example carboxylic acids such as formic acid, acetic acid, propionic acid, oxalic acid, lactic acid or salicylic acid or sulfonic acids such as p-toluenesulfonic acid, with a basic group such as amino, alkylamino, dialkylamino, piperidino, morpholino or pyridino. In such a case, these salts will comprise the conjugated base of the acid as the anion.

Preference is given to compounds of the general formula (I) where the symbols and indices are defined as follows:

Q represents a radical Q1, Q2, Q3 or Q4,

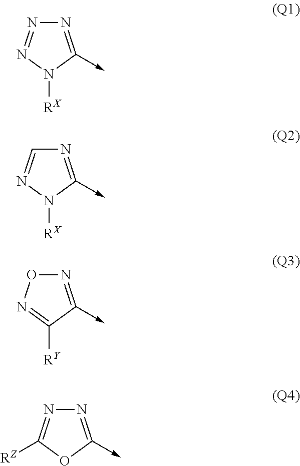

X represents (C₁-C₆)-alkyl or (C₃-C₆)-cycloalkyl,

R represents (C₁-C₆)-alkyl, (C₃-C₆)-cycloalkyl, (C₃-C₆)-cycloalkyl-(C₁-C₆)-alkyl, (C₁-C₆)-alkyl-O—(C₁-C₆)-alkyl,

R^{a} represents hydrogen,

R^{X} represents (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, where the six radicals mentioned above are in each case substituted by s radicals from the group consisting of R²(O)ₙS, (R¹)₂N, R¹O, R¹(O)C, R¹O(O)C, R¹(O)CO, R²O(O)CO, R¹(O)C(R¹)N, R²(O)₂S(R¹)N, (C₃-C₆)-cycloalkyl, heteroaryl, heterocyclyl and phenyl, where the four last-mentioned radicals are substituted by s radicals from the group consisting of (C₁-C₆)-alkyl, halo-(C₁-C₆)-alkyl, (C₁-C₆)-alkoxy and halogen, and where heterocyclyl carries n oxo groups, or R^{X} represents (C₃-C₇)-cycloalkyl, where this radical is substituted by s radicals from the group consisting of halogen, (C₁-C₆)-alkyl and halo-(C₁-C₆)-alkyl,

R^{Y} represents hydrogen, (C₁-C₆)-alkyl, halo-(C₁-C₆)-alkyl, (C₃-C₇)-cycloalkyl, (C₁-C₆)-alkoxy, methoxycarbonyl, methoxycarbonylmethyl, halogen, amino, aminocarbonyl or methoxymethyl,

R^{Z} represents hydrogen, (C₁-C₆)-alkyl, R¹O—(C₁-C₆)-alkyl, R′CH₂, (C₃-C₇)-cycloalkyl, halo-(C₁-C₆)-alkyl, R¹O, R¹(H)N, methoxycarbonyl, acetylamino or methylsulfonyl,

R¹ represents hydrogen, (C₁-C₆)-alkyl, halo-(C₁-C₆)-alkyl, (C₃-C₆)-cycloalkyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, where the nine last-mentioned radicals are in each case substituted by s radicals from the group consisting of nitro, halogen, (C₁-C₆)-alkyl, halo-(C₁-C₆)-alkyl, R³O(O)C, (R³)₂N(O)C, R³O, (R³)₂N, R⁴(O)ₙS and R³O—(C₁-C₆)-alkyl, and where heterocyclyl carries n oxo groups,

R² represents (C₁-C₆)-alkyl, halo-(C₁-C₆)-alkyl, (C₃-C₆)-cycloalkyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, where the nine last-mentioned radicals are in each case substituted by s radicals from the group consisting of nitro, halogen, (C₁-C₆)-alkyl, halo-(C₁-C₆)-alkyl, R³O(O)C, (R³)₂N(O)C, R³O, (R³)₂N, R⁴(O)ₙS and R³O—(C₁-C₆)-alkyl, and where heterocyclyl carries n oxo groups,

R³ represents hydrogen or (C₁-C₆)-alkyl,

R⁴ represents (C₁-C₆)-alkyl,

R′ represents acetoxy, acetamido, methoxycarbonyl or (C₃-C₆)-cycloalkyl,

n represents 0, 1 or 2,

s represents 0, 1, 2 or 3.

Preference is also given to compounds of the general formula (I) where the symbols and indices are defined as follows:

Q represents a radical Q1, Q2, Q3 or Q4,

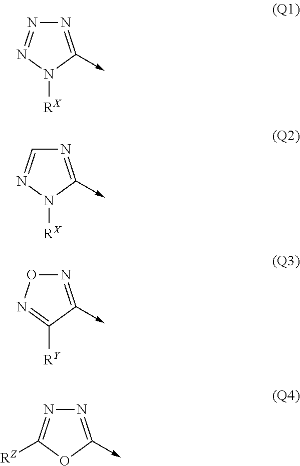

X represents halogen,

R represents (C₁-C₆)-alkyl, (C₃-C₆)-cycloalkyl, (C₃-C₆)-cycloalkyl-(C₁-C₆)-alkyl, (C₁-C₆)-alkyl-O—(C₁-C₆)-alkyl,

R^{a} represents hydrogen,

R^{X} represents (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, where the six radicals mentioned above are in each case substituted by s radicals from the group consisting of R²(O)ₙS, (R¹)₂N, R¹O, R¹(O)C, R¹O(O)C, R¹(O)CO, R²O(O)CO, R¹(O)C(R¹)N, R²(O)₂S(R¹)N, (C₃-C₆)-cycloalkyl, heteroaryl, heterocyclyl and phenyl, where the four last-mentioned radicals are substituted by s radicals from the group consisting of (C₁-C₆)-alkyl, halo-(C₁-C₆)-alkyl, (C₁-C₆)-alkoxy and halogen, and where heterocyclyl carries n oxo groups,

or R^{X} represents (C₃-C₇)-cycloalkyl, where this radical is substituted by s radicals from the group consisting of halogen, (C₁-C₆)-alkyl and halo-(C₁-C₆)-alkyl,

R^{Y} represents hydrogen, (C₁-C₆)-alkyl, halo-(C₁-C₆)-alkyl, (C₃-C₇)-cycloalkyl, (C₁-C₆)-alkoxy, methoxycarbonyl, methoxycarbonylmethyl, halogen, amino, aminocarbonyl or methoxymethyl,

R^{Z} represents hydrogen, (C₁-C₆)-alkyl, R¹O—(C₁-C₆)-alkyl, R′CH₂, (C₃-C₇)-cycloalkyl, halo-(C₁-C₆)-alkyl, R¹O, R¹(H)N, methoxycarbonyl, acetylamino or methylsulfonyl,

R¹ represents hydrogen, (C₁-C₆)-alkyl, halo-(C₁-C₆)-alkyl, (C₃-C₆)-cycloalkyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl (C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, where the nine last-mentioned radicals are in each case substituted by s radicals from the group consisting of nitro, halogen, (C₁-C₆)-alkyl, halo-(C₁-C₆)-alkyl, R³O(O)C, (R³)₂N(O)C, R³O, (R³)₂N, R⁴(O)ₙS and R³O—(C₁-C₆)-alkyl, and where heterocyclyl carries n oxo groups,

R² represents (C₁-C₆)-alkyl, halo-(C₁-C₆)-alkyl, (C₃-C₆)-cycloalkyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, where the nine last-mentioned radicals are in each case substituted by s radicals from the group consisting of nitro, halogen, (C₁-C₆)-alkyl, halo-(C₁-C₆)-alkyl, R³O(O)C, (R³)₂N(O)C, R³O, (R³)₂N, R⁴(O)ₙS and R³O—(C₁-C₆)-alkyl, and where heterocyclyl carries n oxo groups,

R³ represents hydrogen or (C₁-C₆)-alkyl,

R⁴ represents (C₁-C₆)-alkyl,

R′ represents acetoxy, acetamido, methoxycarbonyl or (C₃-C₆)-cycloalkyl,

n represents 0, 1 or 2,

s represents 0, 1, 2 or 3.

Very particular preference is given to compounds of the general formula (I) in which the symbols and indices are defined as follows:

Q represents a radical Q1, Q2, Q3 or Q4,

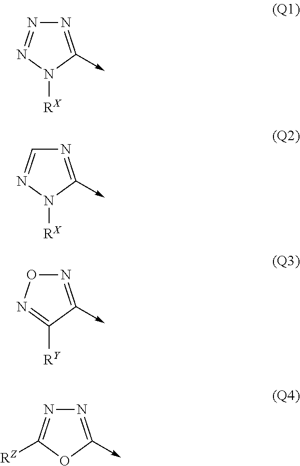

X represents methyl, ethyl or cyclopropyl,

R represents methyl, ethyl, cyclopropylmethyl or methoxyethyl,

R^{a} represents hydrogen,

R^{X} represents methyl, ethyl or n-propyl,

R^{Y} represents methyl or chlorine,

R^{Z} represents methyl,

n represents 0, 1 or 2.

Very particular preference is also given to compounds of the general formula (I) in which the symbols and indices are defined as follows:

Q represents a radical Q1, Q2, Q3 or Q4,

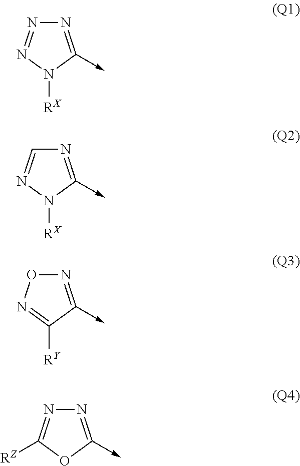

X represents fluorine, chlorine, bromine or iodine,

R represents methyl, ethyl, cyclopropylmethyl or methoxyethyl,

R^{a} represents hydrogen,

R^{X} represents methyl, ethyl or n-propyl,

R^{Y} represents methyl or chlorine,

R^{Z} represents methyl,

n represents 0, 1 or 2.

In all the formulae specified hereinafter, the substituents and symbols have the same meaning as described in formula (I), unless defined differently.

Compounds of the invention in which Q represents Q1 or Q2, and the aminotetrazoles and aminotriazoles that underlie these amides, can be prepared, for example, by the methods specified in WO 2012/028579 A1.

Compounds of the invention in which Q represents Q3, and the aminofurazans that underlie these amides, can be prepared, for example, by the methods specified in WO 2011/035874A1.

Compounds of the invention in which Q is Q4 can be prepared, for example, by the methods specified in WO 2012/126932 A1. The 2-amino-1,3,4-oxadiazoles that underlie these amides are commercially available or synthetically obtainable by standard methods that are known from the literature.

The benzoyl chlorides that underlie the compounds (I) according to the invention, or the corresponding benzoic acids, can be prepared, for example, by the method shown in scheme 1. The 2-hydroxybenzoic esters required for this purpose can be obtained by the process specified in WO 2014/090766 A1 (see in particular synthesis example 2 on p. 6 of that document). The hydroxyl group is methylated, followed by ester hydrolysis. After the formation of the oxazoline group, the methoxy group can be nucleophilically exchanged for alkyl, cycloalkyl or amino groups (A. I. Meyers et al., J. Org. Chem., 1978, 43 (7), 1372-1379; A. I. Meyers et al., J. Org. Chem., 1977, 42 (15), 2653-2654; A. I. Meyers et al., Tetrahedron, 1994, 50 (8), 2297-2360; T. W. Greene, P. G. M. Wuts, Protective Groups in Organic Synthesis, 2nd Edition, John Wiley & Sons, Inc. 1991, p. 265 ff.; Z. Hell et al., Tetrahedron Letters, 2002, 43, 3985-3987.). Subsequent oxazoline cleavage affords the substituted 4-difluoromethylbenzoic acid which can be modified further depending on the substitution pattern desired. For example, 2-aminobenzoic acids can be converted via a Sandmeyer reaction into their 2-halobenzoic acids.

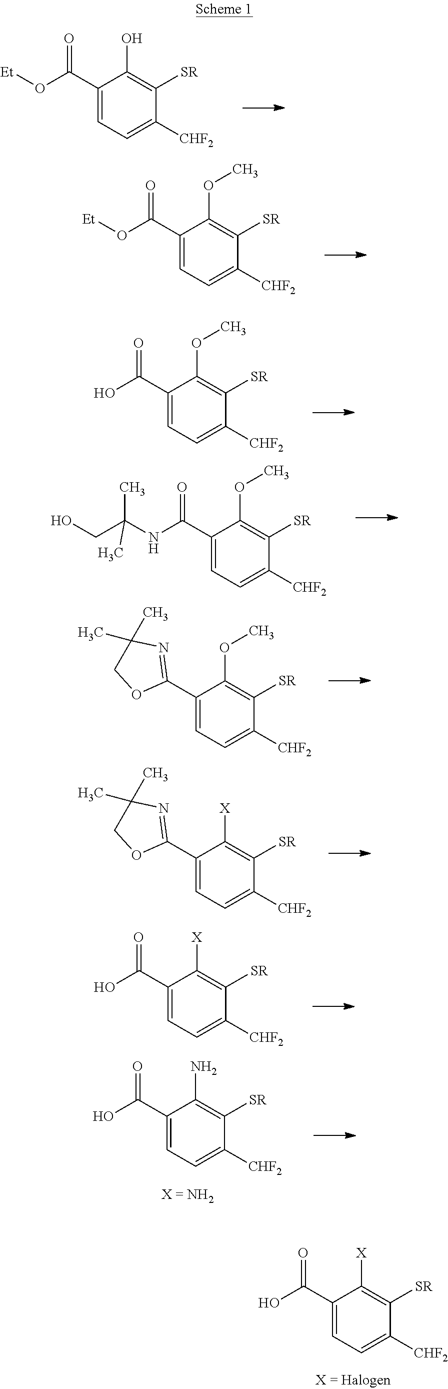

The prior art discloses a number of other methods for introducing a difluoromethyl group, for example: Y. Lu, C. Liu, Q.-Y. Chen, Curr. Org. Chem., 2015, 19, 1638-1650.

The thioether can be oxidized further, for example according to scheme 2, to give the corresponding sulfoxide or sulfone. Oxidation methods that lead selectively to the sulfoxide or sulfone are known from the literature. A number of oxidation systems are suitable, for example peracids such as meta-chloroperbenzoic acid, which are optionally generated in situ (for example peracetic acid in the system acetic acid/hydrogen peroxide/sodium tungstate(VI)) (Houben-Weyl, Methoden der Organischen Chemie [Methods of Organic Chemistry], Georg Thieme Verlag Stuttgart, Vol. E 11, expanded and supplementary volumes to the 4th edition 1985, p. 702 ff., p. 718 ff. and p. 1194 ff.).

The substitution pattern and the oxidizing agent are among the factors that decide the point in the synthesis cascade at which the oxidation of the thioether is appropriate. An oxidation may be appropriate, for example, as shown in scheme 2, at the stage of the free benzoic acid or at the stage of the amide of the formula (I) where R^{a}═H and n=0.

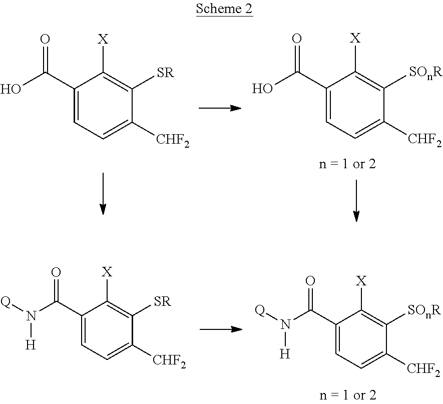

It may be appropriate to alter the sequence of the reaction steps. For instance, benzoic acids bearing a sulfoxide cannot be converted directly to their acid chlorides. One option here is first to prepare the amide of the formula (I) where R^{a}═H and n=0 at the thioether stage and then to oxidize the thioether to the sulfoxide.

The workup of the respective reaction mixtures is generally effected by known processes, for example by crystallization, aqueous-extractive workup, by chromatographic methods or by a combination of these methods.

The preparation of the compounds (I) according to the invention can, as described above, proceed via substituted benzoic acids of the formula (II) or corresponding benzoyl chlorides of the formula (III).

Compounds of the formula (II) are novel and are very well-suited as intermediates for the preparation of the compounds of the formula (I) according to the invention. The present invention therefore further provides compounds of the formula (II)

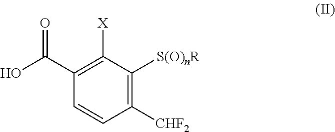

in which the symbols and indices are defined as follows:

X represents (C₃-C₆)-cycloalkyl or halogen,

R represents (C₁-C₆)-alkyl, (C₃-C₆)-cycloalkyl, (C₃-C₆)-cycloalkyl-(C₁-C₆)-alkyl, (C₁-C₆)-alkyl-O—(C₁-C₆)-alkyl,

n represents 0, 1 or 2.

Preference is given to compounds (II) in which

X represents cyclopropyl, fluorine, chlorine, bromine or iodine,

R represents methyl, ethyl, cyclopropylmethyl or methoxyethyl,

n represents 0, 1 or 2.

Compounds of the formula (III) are likewise novel and are very well-suited as intermediates for the preparation of the compounds of the formula (I) according to the invention. The present invention therefore further provides compounds of the formula (III)

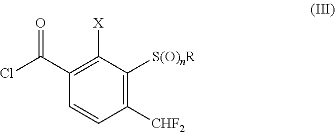

in which the symbols and indices are defined as follows:

X represents (C₁-C₆)-alkyl, (C₃-C₆)-cycloalkyl or halogen,

R represents (C₁-C₆)-alkyl, (C₃-C₆)-cycloalkyl, (C₃-C₆)-cycloalkyl-(C₁-C₆)-alkyl, (C₁-C₆)-alkyl (C₁-C₆)-alkyl,

n represents 0, 1 or 2.

Preference is given to compounds (III) in which

X represents methyl, ethyl, cyclopropyl, fluorine, chlorine, bromine or iodine,

R represents methyl, ethyl, cyclopropylmethyl or methoxyethyl,

n represents 0, 1 or 2.

Collections of compounds of the formula (I) and/or salts thereof which can be synthesized by the abovementioned reactions can also be prepared in a parallelized manner, in which case this may be accomplished in a manual, partly automated or fully automated manner. It is possible, for example, to automate the conduct of the reaction, the workup or the purification of the products and/or intermediates. Overall, this is understood to mean a procedure as described, for example, by D. Tiebes in Combinatorial Chemistry—Synthesis, Analysis, Screening (editor: Gunther Jung), Wiley, 1999, on pages 1 to 34.

For the parallelized conduct of the reaction and workup, it is possible to use a number of commercially available instruments, for example Calypso reaction blocks from Barnstead International, Dubuque, Iowa 52004-0797, USA or reaction stations from Radleys, Shirehill, Saffron Walden, Essex, CB11 3AZ, England, or MultiPROBE Automated Workstations from Perkin Elmer, Waltham, Mass. 02451, USA. For the parallelized purification of compounds of the general formula (I) and salts thereof or of intermediates which occur in the course of preparation, available apparatuses include chromatography apparatuses, for example from ISCO, Inc., 4700 Superior Street, Lincoln, Nebr. 68504, USA.

The apparatuses detailed lead to a modular procedure in which the individual working steps are automated, but manual operations have to be carried out between the working steps. This can be circumvented by using partly or fully integrated automation systems in which the respective automation modules are operated, for example, by robots. Automation systems of this type can be obtained, for example, from Caliper, Hopkinton, Mass. 01748, USA.

The implementation of single or multiple synthesis steps can be supported by the use of polymer-supported reagents/scavenger resins. The specialist literature describes a series of experimental protocols, for example in ChemFiles, Vol. 4, No. 1, Polymer-Supported Scavengers and Reagents for Solution-Phase Synthesis (Sigma-Aldrich).

Aside from the methods described here, compounds of the general formula (I) and salts thereof can be prepared completely or partially by solid-phase-supported methods. For this purpose, individual intermediates or all intermediates in the synthesis or a synthesis adapted for the corresponding procedure are bound to a synthesis resin. Solid-phase-supported synthesis methods are described adequately in the technical literature, for example Barry A. Bunin in “The Combinatorial Index”, Academic Press, 1998 and Combinatorial Chemistry—Synthesis, Analysis, Screening (editor: Gunther Jung), Wiley, 1999. The use of solid-phase-supported synthesis methods permits a number of protocols, which are known from the literature and which for their part may be performed manually or in an automated manner. The reactions can be performed, for example, by means of IRORI technology in microreactors from Nexus Biosystems, 12140 Community Road, Poway, Calif. 92064, USA.

Both in the solid and in the liquid phase, the implementation of individual or several synthesis steps may be supported by the use of microwave technology. The specialist literature describes a series of experimental protocols, for example in Microwaves in Organic and Medicinal Chemistry (editors: C. O. Kappe and A. Stadler), Wiley, 2005.

The preparation by the processes described here gives compounds of the formula (I) and salts thereof in the form of substance collections, which are called libraries. The present invention also provides libraries comprising at least two compounds of the formula (I) and salts thereof.

The compounds of the invention have excellent herbicidal efficacy against a broad spectrum of economically important mono- and dicotyledonous annual harmful plants. The active compounds also act efficiently on perennial weeds which produce shoots from rhizomes, root stocks and other perennial organs and which are difficult to control.

The present invention therefore also provides a method for controlling unwanted plants or for regulating the growth of plants, preferably in plant crops, in which one or more compound(s) of the invention is/are applied to the plants (for example harmful plants such as monocotyledonous or dicotyledonous weeds or unwanted crop plants), the seed (for example grains, seeds or vegetative propagules such as tubers or shoot parts with buds) or the area on which the plants grow (for example the area under cultivation). The compounds of the invention can be deployed, for example, prior to sowing (if appropriate also by incorporation into the soil), prior to emergence or after emergence. Specific examples of some representatives of the monocotyledonous and dicotyledonous weed flora which can be controlled by the compounds of the invention are as follows, though there is no intention to restrict the enumeration to particular species.

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria and Sorghum.

Dicotyledonous weeds of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola and Xanthium.

If the compounds of the invention are applied to the soil surface before germination, either the emergence of the weed seedlings is prevented completely or the weeds grow until they have reached the cotyledon stage, but then they stop growing and ultimately die completely after three to four weeks have passed.

If the active compounds are applied post-emergence to the green parts of the plants, growth stops after the treatment, and the harmful plants remain at the growth stage at the time of application, or they die completely after a certain time, so that in this manner competition by the weeds, which is harmful to the crop plants, is eliminated very early and in a sustained manner.

Although the compounds of the invention have outstanding herbicidal activity against monocotyledonous and dicotyledonous weeds, crop plants of economically important crops, for example dicotyledonous crops of the genera Arachis, Beta, Brassica, Cucumis, Cucurbita, Helianthus, Daucus, Glycine, Gossypium, Ipomoea, Lactuca, Linum, Lycopersicon, Miscanthus, Nicotiana, Phaseolus, Pisum, Solanum, Vicia, or monocotyledonous crops of the genera Allium, Ananas, Asparagus, Avena, Hordeum, Oryza, Panicum, Saccharum, Secale, Sorghum, Triticale, Triticum, Zea, in particular Zea and Triticum, will be damaged to a negligible extent only, if at all, depending on the structure of the particular compound of the invention and its application rate. For these reasons, the present compounds are very suitable for selective control of unwanted plant growth in plant crops such as agriculturally useful plants or ornamental plants.

In addition, the compounds of the invention, depending on their particular chemical structure and the application rate deployed, have outstanding growth-regulating properties in crop plants. They intervene in the plants' own metabolism with regulatory effect, and can thus be used for the controlled influencing of plant constituents and to facilitate harvesting, for example by triggering desiccation and stunted growth. In addition, they are also suitable for general control and inhibition of unwanted vegetative growth without killing the plants. Inhibition of vegetative growth plays a major role for many mono- and dicotyledonous crops since, for example, this can reduce or completely prevent lodging.

By virtue of their herbicidal and plant growth regulatory properties, the active compounds can also be used to control harmful plants in crops of genetically modified plants or plants modified by conventional mutagenesis. In general, the transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material.

It is preferable with a view to transgenic crops to use the compounds of the invention in economically important transgenic crops of useful plants and ornamentals, for example of cereals such as wheat, barley, rye, oats, millet/sorghum, rice and corn or else crops of sugar beet, cotton, soybean, oilseed rape, potato, manioc, tomato, peas and other vegetables.

Preferably, the compounds of the invention can be used as herbicides in crops of useful plants which are resistant, or have been made resistant by genetic engineering, to the phytotoxic effects of the herbicides.

Conventional ways of producing novel plants which have modified properties in comparison to existing plants consist, for example, in traditional cultivation methods and the generation of mutants. Alternatively, novel plants with modified properties can be generated with the aid of recombinant methods (see, for example, EP-A-0221044, EP-A-0131624). For example, there have been descriptions in several cases of:
genetic modifications of crop plants for the purpose of modifying the starch synthesized in the plants (for example WO 92/11376, WO 92/14827, WO 91/19806),
transgenic crop plants which are resistant to particular herbicides of the glufosinate type (cf., for example, EP-A-0242236, EP-A-242246) or glyphosate type (WO 92/00377) or of the sulfonylurea type (EP-A-0257993, U.S. Pat. No. 5,013,659),
transgenic crop plants, for example cotton, which is capable of producing Bacillus thuringiensis toxins (Bt toxins), which make the plants resistant to certain pests (EP-A-0142924, EP-A-0193259),
transgenic crop plants having a modified fatty acid composition (WO 91/13972),
genetically modified crop plants with novel constituents or secondary metabolites, for example novel phytoalexins, which bring about an increased disease resistance (EPA 309862, EPA0464461),
genetically modified plants having reduced photorespiration, which have higher yields and higher stress tolerance (EPA 0305398),
transgenic crop plants which produce pharmaceutically or diagnostically important proteins (“molecular pharming”),
transgenic crop plants which feature higher yields or better quality,
transgenic crop plants which feature a combination, for example, of the abovementioned novel properties (“gene stacking”).

Numerous molecular biology techniques which can be used to produce novel transgenic plants with modified properties are known in principle; see, for example, I. Potrykus and G. Spangenberg (eds.) Gene Transfer to Plants, Springer Lab Manual (1995), Springer Verlag Berlin, Heidelberg, or Christou, “Trends in Plant Science” 1 (1996) 423-431.

For such genetic manipulations, nucleic acid molecules which allow mutagenesis or sequence alteration by recombination of DNA sequences can be introduced into plasmids. With the aid of standard methods, it is possible, for example, to undertake base exchanges, remove parts of sequences or add natural or synthetic sequences. To join the DNA fragments with one another, adapters or linkers can be placed onto the fragments, see, for example, Sambrook et al., 1989, Molecular Cloning, A Laboratory Manual, 2nd edition, Cold Spring Harbor Laboratory Press, Cold Spring Harbor, N.Y., or Winnacker “Gene and Klone” [Genes and clones], VCH Weinheim 2nd edition 1996.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product. To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Such sequences are known to those skilled in the art (see, for example, Braun et al., EMBO J. 11 (1992), 3219-3227, Wolter et al., Proc. Natl. Acad. Sci. USA 85 (1988), 846-850; Sonnewald et al., Plant J. 1 (1991), 95-106). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants.

Thus, transgenic plants can be obtained whose properties are altered by overexpression, suppression or inhibition of homologous (=natural) genes or gene sequences or expression of heterologous (=foreign) genes or gene sequences.

The compounds of the invention can be used with preference in transgenic crops which are resistant to growth regulators, for example dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, the glyphosates, glufosinates or benzoylisoxazoles and analogous active compounds.

When the active compounds of the invention are employed in transgenic crops, not only do the effects towards harmful plants observed in other crops occur, but frequently also effects which are specific to the application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also provides for the use of the compounds of the invention as herbicides for control of harmful plants in transgenic crop plants.

The compounds of the invention can be applied in the form of wettable powders, emulsifiable concentrates, sprayable solutions, dusting products or granules in the customary formulations. The invention therefore also provides herbicidal and plant-growth-regulating compositions which comprise the compounds of the invention.

The compounds of the invention can be formulated in various ways, according to the biological and/or physicochemical parameters required. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes. These individual formulation types are known in principle and are described, for example, in: Winnacker-Küchler, “Chemische Technologie” [Chemical Engineering], volume 7, C. Hanser Verlag Munich, 4th Ed. 1986, Wade van Valkenburg, “Pesticide Formulations”, Marcel Dekker, N.Y., 1973, K. Martens, “Spray Drying” Handbook, 3rd Ed. 1979, G. Goodwin Ltd. London.

The formulation auxiliaries required, such as inert materials, surfactants, solvents and further additives, are likewise known and are described, for example, in: Watkins, “Handbook of Insecticide Dust Diluents and Carriers”, 2nd Ed., Darland Books, Caldwell N.J.; H. v. Olphen, “Introduction to Clay Colloid Chemistry”, 2nd Ed., J. Wiley & Sons, N.Y.; C. Marsden, “Solvents Guide”, 2nd Ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [Interface-active Ethylene Oxide Adducts], Wiss. Verlagsgesellschaft, Stuttgart 1976; Winnacker-Küchler, “Chemische Technologie” [Chemical Engineering], volume 7, C. Hanser Verlag Munich, 4th Ed. 1986.

Wettable powders are preparations uniformly dispersible in water which, alongside the active compound apart from a diluent or inert substance, also comprise surfactants of an ionic and/or nonionic type (wetting agent, dispersant), e.g. polyethoxylated alkylphenols, polyethoxylated fatty alcohols, polyethoxylated fatty amines, fatty alcohol polyglycolethersulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the herbicidally active compounds are finely ground, for example in customary apparatuses such as hammer mills, blower mills and air-jet mills, and simultaneously or subsequently mixed with the formulation auxiliaries.

Emulsifiable concentrates are produced by dissolving the active compound in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulfonates such as calcium dodecylbenzenesulfonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters, or polyoxyethylene sorbitan esters, for example polyoxyethylene sorbitan fatty acid esters.

Dusting products are obtained by grinding the active compound with finely distributed solids, for example talc, natural clays, such as kaolin, bentonite and pyrophyllite, or diatomaceous earth.

Suspension concentrates may be water- or oil-based. They may be prepared, for example, by wet-grinding by means of commercial bead mills and optional addition of surfactants as have, for example, already been listed above for the other formulation types.

Emulsions, for example oil-in-water emulsions (EW), can be produced, for example, by means of stirrers, colloid mills and/or static mixers using aqueous organic solvents and optionally surfactants as already listed above, for example, for the other formulation types.

Granules can be produced either by spraying the active compound onto adsorptive granular inert material or by applying active compound concentrates to the surface of carriers, such as sand, kaolinites or granular inert material, by means of adhesives, for example polyvinyl alcohol, sodium polyacrylate or else mineral oils. Suitable active compounds can also be granulated in the manner customary for the production of fertilizer granules—if desired as a mixture with fertilizers.

Water-dispersible granules are produced generally by the customary processes such as spray-drying, fluidized-bed granulation, pan granulation, mixing with high-speed mixers and extrusion without solid inert material.

For the production of pan, fluidized-bed, extruder and spray granules, see e.g. processes in “Spray-Drying Handbook” 3rd Ed. 1979, G. Goodwin Ltd., London, J. E. Browning, “Agglomeration”, Chemical and Engineering 1967, pages 147 ff.; “Perry's Chemical Engineer's Handbook”, 5th Ed., McGraw-Hill, New York 1973, pp. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical preparations contain generally 0.1 to 99% by weight, especially 0.1 to 95% by weight, of compounds of the invention.

In wettable powders, the active compound concentration is, for example, about 10 to 90% by weight, the remainder to 100% by weight consisting of customary formulation constituents. In emulsifiable concentrates, the active compound concentration may be about 1% to 90% and preferably 5% to 80% by weight. Dust-type formulations contain

1% to 30% by weight of active compound, preferably usually 5% to 20% by weight of active compound; sprayable solutions contain about 0.05% to 80% by weight, preferably 2% to 50% by weight of active compound. In the case of water-dispersible granules, the active compound content depends partially on whether the active compound is in liquid or solid form and on which granulation auxiliaries, fillers, etc., are used. In the water-dispersible granules, the content of active compound is, for example, between 1% and 95% by weight, preferably between 10% and 80% by weight.

In addition, the active compound formulations mentioned optionally comprise the respective customary stickers, wetters, dispersants, emulsifiers, penetrants, preservatives, antifreeze agents and solvents, fillers, carriers and dyes, defoamers, evaporation inhibitors and agents which influence the pH and the viscosity.

On the basis of these formulations, it is also possible to produce combinations with other pesticidally active substances, for example insecticides, acaricides, herbicides, fungicides, and also with safeners, fertilizers and/or growth regulators, for example in the form of a finished formulation or as a tank mix.

For application, the formulations in commercial form are, if appropriate, diluted in a customary manner, for example in the case of wettable powders, emulsifiable concentrates, dispersions and water-dispersible granules with water. Dust-type preparations, granules for soil application or granules for scattering and sprayable solutions are not normally diluted further with other inert substances prior to application.

The required application rate of the compounds of the formula (I) varies with the external conditions, including, inter alia, temperature, humidity and the type of herbicide used. It can vary within wide limits, for example between 0.001 and 1.0 kg/ha or more of active substance, but it is preferably between 0.005 and 750 g/ha.

The examples which follow illustrate the invention.

### Chemical Examples

### Synthesis of 4-(difluoromethyl)-2-methyl-3-(methylsulfanyl)-N-(1-methyl-1H-1,2,4-triazol-5-yl)benzamide (Example No. 4-1)

### Step 1: Synthesis of ethyl 4-difluoromethyl-2-methoxy-3-methylthiobenzoate

66.02 g (477.7 mmol) of potassium carbonate were added to 104.4 g (398.1 mmol) of ethyl 4-difluoromethyl-2-hydroxy-3-methylthiobenzoate in 310 ml of acetone. A mixture of 6.04 g (59.7 mmol) of triethylamine and 7.53 g (59.7 mmol) of dimethyl sulfate was then added. 57.74 g (457.8 mmol) of dimethyl sulfate were then added dropwise. The reaction mixture was then stirred at room temperature (RT) for 16 h. For work-up the reaction mixture was freed from the solvent and the residue was stirred with 1000 ml of 1M aqueous sodium hydroxide solution for 2 h. CH₂Cl₂ was added to the mixture and, after phase separation, the organic phase was dried. The filtrate was freed of the solvent. 104.7 g of the desired product were obtained as residue.

### Step 2: Synthesis of 4-difluoromethyl-2-methoxy-3-methylthiobenzoic Acid

104.7 g (378.9 mmol) of ethyl 4-difluoromethyl-2-methoxy-3-methylthiobenzoate were stirred with a mixture of 420 ml of 1M aqueous sodium hydroxide solution and 715 ml of methanol at RT for 16 h. For workup, the methanol was removed. The residue was extracted with ethyl acetate and the aqueous phase was then acidified with hydrochloric acid. The mixture was then extracted twice with ethyl acetate. The combined organic phases were dried and the filtrate was freed of the solvent. 90.0 g of the desired product were obtained as residue.

### Step 3: Synthesis of 2-[4-(difluoromethyl)-2-methoxy-3-(methylsulfanyl)phenyl]-4,4-dimethyl-4,5-dihydro-1,3-oxazole

1.24 ml (16.1 mmol) of N,N-dimethylformamide were added to 40.0 g (161.1 mmol) of 4-difluoromethyl-2-methoxy-3-methylthiobenzoate in 600 ml of dichloromethane. 23.2 ml (265.9 mmol) of oxalyl chloride were then added dropwise. The reaction mixture was stirred at RT for 16 h. To bring the reaction to completion, 2.81 ml (32.2 mmol) of oxalyl chloride were then added and the mixture was stirred at RT for a further 24 h. The content was then concentrated and the residue was re-dissolved in 300 ml of CH₂Cl₂. Subsequently, a solution of 15.08 g (169.2 mmol) of 2-amino-2-methyl-1-propanol in 10% strength aqueous sodium hydroxide solution was added dropwise with slight ice bath cooling. The mixture was stirred at RT for 24 h. For work-up, the mixture was diluted first with CH₂Cl₂ and then with a little water. After phase separation, the aqueous phase was extracted with CH₂Cl₂. The combined organic phases were extracted and the filtrate was concentrated. For the second reaction step, the residue was dissolved in 600 ml of CH₂Cl₂, and 32.9 ml (451.2 mmol) of thionyl chloride were added. The reaction mixture was then stirred at RT for 16 h. For work-up, 500 ml of 10% strength aqueous sodium hydroxide solution were added over 2 h with ice bath cooling, followed by the addition of a further 100 ml of 10% strength aqueous sodium hydroxide solution. After phase separation, the aqueous phase was extracted with CH₂Cl₂. The combined organic phases were dried and the filtrate was concentrated. 200 ml of 6M hydrochloric acid were added to the residue and the mixture was extracted three times with in each case 60 ml of CH₂Cl₂. The combined organic phases were extracted twice with in each case 25 ml of 6M hydrochloric acid and then twice with in each case 20 ml of 6M hydrochloric acid. The hydrochloric acid phases were cooled in an ice bath and made alkaline with solid NaOH added a little at a time. The mixture was then extracted twice with in each case 200 ml of CH₂Cl₂ and then once more with 100 ml of CH₂Cl₂. The organic phases were dried and the filtrate was freed of the solvent. 32.6 g of the desired product were obtained as residue.

### Step 4: Synthesis of 2-[4-(difluoromethyl)-2-methyl-3-(methylsulfanyl)phenyl]-4,4-dimethyl-4,5-dihydro-1,3-oxazole

At RT, 84.6 ml of a 1M solution (84.6 mmol) of methylmagnesium bromide in THF were added dropwise to a solution of 17.0 g (56.4 mmol) of 2-[4-(difluoromethyl)-2-methoxy (methylsulfanyl)phenyl]-4,4-dimethyl-4,5-dihydro-1,3-oxazole in 280 ml of diethyl ether. After 2 h, a further 56 ml of the 1M solution (56 mmol) of methylmagnesium bromide in THF were added dropwise over 3 h. The mixture was stirred at RT for 72 h. For work-up, the content was carefully poured onto a mixture of ice and dilute hydrochloric acid. The mixture was neutralized with NaOH and extracted twice with diethyl ether. The organic phases were dried and the filtrate was freed of the solvent. 15.9 g of the desired product were obtained as residue.

### Step 5: Synthesis of 4-difluoromethyl-2-methyl-3-methylthiobenzoic Acid

30.9 ml (497 mmol) of iodomethane were added to 15.47 g (54.2 mmol) of 2-[4-(difluoromethyl)-2-methyl-3-(methylsulfanyl)phenyl]-4,4-dimethyl-4,5-dihydro-1,3-oxazole in 250 ml of acetone and the mixture was then stirred at 40° C. for 2 h. A further 25 ml (402 mmol) of iodomethane were then added and the mixture was subsequently stirred at a temperature of 40° C. for 20 h. To bring the reaction to completion, another 10 ml (161 mmol) of iodomethane were then added and the mixture was subsequently stirred at a temperature of 40° C. for 15 h. For work-up, the reaction mixture was cooled to RT and concentrated. For the reaction of the second reaction step, 150 ml of methanol and 150 ml of 20% strength aqueous sodium hydroxide solution were added to the residue and the mixture was heated under reflux for 2 h. Finally, the reaction mixture was stirred at RT for 72 h. For work-up, the contents were concentrated and the residue was taken up in a little water. The mixture was washed with CH₂Cl₂ and the aqueous phase was acidified with concentrated hydrochloric acid. The mixture was then extracted with CH₂Cl₂. The organic phase was freed from the solvent. 11.8 g of the desired product were obtained as residue.

### Step 6: Synthesis of 4-(difluoromethyl)-2-methyl-3-(methylsulfanyl)-N-(1-methyl-1H-1,2,4-triazol-5-yl)benzamide (No. 4-1)

At RT, 137.4 mg (1.4 mmol) of 1-methyl-1H-1,2,4-triazole-5-amine were added to 232.2 mg (1.0 mmol) of 4-difluoromethyl-2-methyl-3-methylthiobenzoic acid in 5 ml of pyridine. With cooling, 177.7 mg (1.4 mmol) of oxalyl chloride were added and the mixture was then stirred at RT for 16 h. For work-up, the mixture was concentrated and the residue was taken up in CH₂Cl₂. The mixture was extracted once with an aqueous NaHCO₃ solution and then extracted once with water. The organic phase was dried and the filtrate was concentrated. The residue was purified by chromatography, and 62.6 mg of the desired product were isolated.

The examples listed in the tables below were prepared analogously to the methods mentioned above or can be obtained analogously to the methods mentioned above. These compounds are very particularly preferred.

The abbreviations used mean:

Ph=phenyl Me=methyl Et=ethyl c-Pr=cyclopropyl

TABLE 1
Compounds of the general formula (I) according to the
invention in which Q represents Q1 and R^{x} represents
a methyl group and R^{a} represents hydrogen

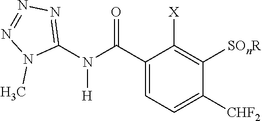

| No. | X | n | R |
|---|---|---|---|
| 1-1 | Me | 0 | Me |
| 1-2 | Me | 1 | Me |
| 1-3 | Me | 2 | Me |
| 1-4 | Me | 0 | Et |
| 1-5 | Me | 1 | Et |
| 1-6 | Me | 2 | Et |
| 1-7 | Me | 0 | CH₂c-Pr |
| 1-8 | Me | 1 | CH₂c-Pr |
| 1-9 | Me | 2 | CH₂c-Pr |
| 1-10 | Me | 0 | CH₂CH₂OMe |
| 1-11 | Me | 1 | CH₂CH₂OMe |
| 1-12 | Me | 2 | CH₂CH₂OMe |
| 1-13 | Et | 0 | Me |
| 1-14 | Et | 1 | Me |
| 1-15 | Et | 2 | Me |
| 1-16 | Et | 0 | Et |
| 1-17 | Et | 1 | Et |
| 1-18 | Et | 2 | Et |
| 1-19 | Et | 0 | CH₂c-Pr |
| 1-20 | Et | 1 | CH₂c-Pr |
| 1-21 | Et | 2 | CH₂c-Pr |
| 1-22 | Et | 0 | CH₂CH₂OMe |
| 1-23 | Et | 1 | CH₂CH₂OMe |
| 1-24 | Et | 2 | CH₂CH₂OMe |
| 1-25 | c-Pr | 0 | Me |
| 1-26 | c-Pr | 1 | Me |
| 1-27 | c-Pr | 2 | Me |
| 1-28 | c-Pr | 0 | Et |
| 1-29 | c-Pr | 1 | Et |
| 1-30 | c-Pr | 2 | Et |
| 1-31 | c-Pr | 0 | CH₂c-Pr |
| 1-32 | c-Pr | 1 | CH₂c-Pr |
| 1-33 | c-Pr | 2 | CH₂c-Pr |
| 1-34 | c-Pr | 0 | CH₂CH₂OMe |
| 1-35 | c-Pr | 1 | CH₂CH₂OMe |
| 1-36 | c-Pr | 2 | CH₂CH₂OMe |
| 1-37 | F | 0 | Me |
| 1-38 | F | 1 | Me |
| 1-39 | F | 2 | Me |
| 1-40 | F | 0 | Et |
| 1-41 | F | 1 | Et |
| 1-42 | F | 2 | Et |
| 1-43 | F | 0 | CH₂c-Pr |
| 1-44 | F | 1 | CH₂c-Pr |
| 1-45 | F | 2 | CH₂c-Pr |
| 1-46 | F | 0 | CH₂CH₂OMe |
| 1-47 | F | 1 | CH₂CH₂OMe |
| 1-48 | F | 2 | CH₂CH₂OMe |
| 1-49 | Cl | 0 | Me |
| 1-50 | Cl | 1 | Me |
| 1-51 | Cl | 2 | Me |
| 1-52 | Cl | 0 | Et |
| 1-53 | Cl | 1 | Et |
| 1-54 | Cl | 2 | Et |
| 1-55 | Cl | 0 | CH₂c-Pr |
| 1-56 | Cl | 1 | CH₂c-Pr |
| 1-57 | Cl | 2 | CH₂c-Pr |
| 1-58 | Cl | 0 | CH₂CH₂OMe |
| 1-59 | Cl | 1 | CH₂CH₂OMe |
| 1-60 | Cl | 2 | CH₂CH₂OMe |
| 1-61 | Br | 0 | Me |
| 1-62 | Br | 1 | Me |
| 1-63 | Br | 2 | Me |
| 1-64 | Br | 0 | Et |
| 1-65 | Br | 1 | Et |
| 1-66 | Br | 2 | Et |
| 1-67 | Br | 0 | CH₂c-Pr |
| 1-68 | Br | 1 | CH₂c-Pr |
| 1-69 | Br | 2 | CH₂c-Pr |
| 1-70 | Br | 0 | CH₂CH₂OMe |
| 1-71 | Br | 1 | CH₂CH₂OMe |
| 1-72 | Br | 2 | CH₂CH₂OMe |
| 1-73 | I | 0 | Me |
| 1-74 | I | 1 | Me |
| 1-75 | I | 2 | Me |
| 1-76 | I | 0 | Et |
| 1-77 | I | 1 | Et |
| 1-78 | I | 2 | Et |
| 1-79 | I | 0 | CH₂c-Pr |
| 1-80 | I | 1 | CH₂c-Pr |
| 1-81 | I | 2 | CH₂c-Pr |
| 1-82 | I | 0 | CH₂CH₂OMe |
| 1-83 | I | 1 | CH₂CH₂OMe |
| 1-84 | I | 2 | CH₂CH₂OMe |

TABLE 2
Compounds of the general formula (I) according to the
invention in which Q represents Q1 and R^{x} represents
an ethyl group and R^{a} represents hydrogen.

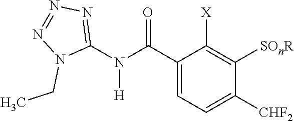

| No. | X | n | R |
|---|---|---|---|
| 2-1 | Me | 0 | Me |
| 2-2 | Me | 1 | Me |
| 2-3 | Me | 2 | Me |
| 2-4 | Me | 0 | Et |
| 2-5 | Me | 1 | Et |
| 2-6 | Me | 2 | Et |
| 2-7 | Me | 0 | CH₂c-Pr |
| 2-8 | Me | 1 | CH₂c-Pr |
| 2-9 | Me | 2 | CH₂c-Pr |
| 2-10 | Me | 0 | CH₂CH₂OMe |
| 2-11 | Me | 1 | CH₂CH₂OMe |
| 2-12 | Me | 2 | CH₂CH₂OMe |
| 2-13 | Et | 0 | Me |
| 2-14 | Et | 1 | Me |
| 2-15 | Et | 2 | Me |
| 2-16 | Et | 0 | Et |
| 2-17 | Et | 1 | Et |
| 2-18 | Et | 2 | Et |
| 2-19 | Et | 0 | CH₂c-Pr |
| 2-20 | Et | 1 | CH₂c-Pr |
| 2-21 | Et | 2 | CH₂c-Pr |
| 2-22 | Et | 0 | CH₂CH₂OMe |
| 2-23 | Et | 1 | CH₂CH₂OMe |
| 2-24 | Et | 2 | CH₂CH₂OMe |
| 2-25 | c-Pr | 0 | Me |
| 2-26 | c-Pr | 1 | Me |
| 2-27 | c-Pr | 2 | Me |
| 2-28 | c-Pr | 0 | Et |
| 2-29 | c-Pr | 1 | Et |
| 2-30 | c-Pr | 2 | Et |
| 2-31 | c-Pr | 0 | CH₂c-Pr |
| 2-32 | c-Pr | 1 | CH₂c-Pr |
| 2-33 | c-Pr | 2 | CH₂c-Pr |
| 2-34 | c-Pr | 0 | CH₂CH₂OMe |
| 2-35 | c-Pr | 1 | CH₂CH₂OMe |
| 2-36 | c-Pr | 2 | CH₂CH₂OMe |
| 2-37 | F | 0 | Me |
| 2-38 | F | 1 | Me |
| 2-39 | F | 2 | Me |
| 2-40 | F | 0 | Et |
| 2-41 | F | 1 | Et |
| 2-42 | F | 2 | Et |
| 2-43 | F | 0 | CH₂c-Pr |
| 2-44 | F | 1 | CH₂c-Pr |
| 2-45 | F | 2 | CH₂c-Pr |
| 2-46 | F | 0 | CH₂CH₂OMe |
| 2-47 | F | 1 | CH₂CH₂OMe |
| 2-48 | F | 2 | CH₂CH₂OMe |
| 2-49 | Cl | 0 | Me |
| 2-50 | Cl | 1 | Me |
| 2-51 | Cl | 2 | Me |
| 2-52 | Cl | 0 | Et |
| 2-53 | Cl | 1 | Et |
| 2-54 | Cl | 2 | Et |
| 2-55 | Cl | 0 | CH₂c-Pr |
| 2-56 | Cl | 1 | CH₂c-Pr |
| 2-57 | Cl | 2 | CH₂c-Pr |
| 2-58 | Cl | 0 | CH₂CH₂OMe |
| 2-59 | Cl | 1 | CH₂CH₂OMe |
| 2-60 | Cl | 2 | CH₂CH₂OMe |
| 2-61 | Br | 0 | Me |
| 2-62 | Br | 1 | Me |
| 2-63 | Br | 2 | Me |
| 2-64 | Br | 0 | Et |
| 2-65 | Br | 1 | Et |
| 2-66 | Br | 2 | Et |
| 2-67 | Br | 0 | CH₂c-Pr |
| 2-68 | Br | 1 | CH₂c-Pr |
| 2-69 | Br | 2 | CH₂c-Pr |
| 2-70 | Br | 0 | CH₂CH₂OMe |
| 2-71 | Br | 1 | CH₂CH₂OMe |
| 2-72 | Br | 2 | CH₂CH₂OMe |
| 2-73 | I | 0 | Me |
| 2-74 | I | 1 | Me |
| 2-75 | I | 2 | Me |
| 2-76 | I | 0 | Et |
| 2-77 | I | 1 | Et |
| 2-78 | I | 2 | Et |
| 2-79 | I | 0 | CH₂c-Pr |
| 2-80 | I | 1 | CH₂c-Pr |
| 2-81 | I | 2 | CH₂c-Pr |
| 2-82 | I | 0 | CH₂CH₂OMe |
| 2-83 | I | 1 | CH₂CH₂OMe |
| 2-84 | I | 2 | CH₂CH₂OMe |

TABLE 3
Compounds of the general formula (I) according to the
invention in which Q represents Q1 and R^{x} represents
an n-propyl group and R^{a} represents hydrogen,

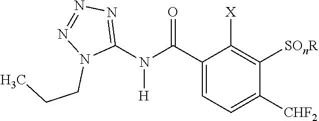

| No. | X | n | R |
|---|---|---|---|
| 3-1 | Me | 0 | Me |
| 3-2 | Me | 1 | Me |
| 3-3 | Me | 2 | Me |
| 3-4 | Me | 0 | Et |
| 3-5 | Me | 1 | Et |
| 3-6 | Me | 2 | Et |
| 3-7 | Me | 0 | CH₂c-Pr |
| 3-8 | Me | 1 | CH₂c-Pr |
| 3-9 | Me | 2 | CH₂c-Pr |
| 3-10 | Me | 0 | CH₂CH₂OMe |
| 3-11 | Me | 1 | CH₂CH₂OMe |
| 3-12 | Me | 2 | CH₂CH₂OMe |
| 3-13 | Et | 0 | Me |
| 3-14 | Et | 1 | Me |
| 3-15 | Et | 2 | Me |
| 3-16 | Et | 0 | Et |
| 3-17 | Et | 1 | Et |
| 3-18 | Et | 2 | Et |
| 3-19 | Et | 0 | CH₂c-Pr |
| 3-20 | Et | 1 | CH₂c-Pr |
| 3-21 | Et | 2 | CH₂c-Pr |
| 3-22 | Et | 0 | CH₂CH₂OMe |
| 3-23 | Et | 1 | CH₂CH₂OMe |
| 3-24 | Et | 2 | CH₂CH₂OMe |
| 3-25 | c-Pr | 0 | Me |
| 3-26 | c-Pr | 1 | Me |
| 3-27 | c-Pr | 2 | Me |
| 3-28 | c-Pr | 0 | Et |
| 3-29 | c-Pr | 1 | Et |
| 3-30 | c-Pr | 2 | Et |
| 3-31 | c-Pr | 0 | CH₂c-Pr |
| 3-32 | c-Pr | 1 | CH₂c-Pr |
| 3-33 | c-Pr | 2 | CH₂c-Pr |
| 3-34 | c-Pr | 0 | CH₂CH₂OMe |
| 3-35 | c-Pr | 1 | CH₂CH₂OMe |
| 3-36 | c-Pr | 2 | CH₂CH₂OMe |
| 3-37 | F | 0 | Me |
| 3-38 | F | 1 | Me |
| 3-39 | F | 2 | Me |
| 3-40 | F | 0 | Et |
| 3-41 | F | 1 | Et |
| 3-42 | F | 2 | Et |
| 3-43 | F | 0 | CH₂c-Pr |
| 3-44 | F | 1 | CH₂c-Pr |
| 3-45 | F | 2 | CH₂c-Pr |
| 3-46 | F | 0 | CH₂CH₂OMe |
| 3-47 | F | 1 | CH₂CH₂OMe |
| 3-48 | F | 2 | CH₂CH₂OMe |
| 3-49 | Cl | 0 | Me |
| 3-50 | Cl | 1 | Me |
| 3-51 | Cl | 2 | Me |
| 3-52 | Cl | 0 | Et |
| 3-53 | Cl | 1 | Et |
| 3-54 | Cl | 2 | Et |
| 3-55 | Cl | 0 | CH₂c-Pr |
| 3-56 | Cl | 1 | CH₂c-Pr |
| 3-57 | Cl | 2 | CH₂c-Pr |
| 3-58 | Cl | 0 | CH₂CH₂OMe |
| 3-59 | Cl | 1 | CH₂CH₂OMe |
| 3-60 | Cl | 2 | CH₂CH₂OMe |
| 3-61 | Br | 0 | Me |
| 3-62 | Br | 1 | Me |
| 3-63 | Br | 2 | Me |
| 3-64 | Br | 0 | Et |
| 3-65 | Br | 1 | Et |
| 3-66 | Br | 2 | Et |
| 3-67 | Br | 0 | CH₂c-Pr |
| 3-68 | Br | 1 | CH₂c-Pr |
| 3-69 | Br | 2 | CH₂c-Pr |
| 3-70 | Br | 0 | CH₂CH₂OMe |
| 3-71 | Br | 1 | CH₂CH₂OMe |
| 3-72 | Br | 2 | CH₂CH₂OMe |
| 3-73 | I | 0 | Me |
| 3-74 | I | 1 | Me |
| 3-75 | I | 2 | Me |
| 3-76 | I | 0 | Et |
| 3-77 | I | 1 | Et |
| 3-78 | I | 2 | Et |
| 3-79 | I | 0 | CH₂c-Pr |
| 3-80 | I | 1 | CH₂c-Pr |
| 3-81 | I | 2 | CH₂c-Pr |
| 3-82 | I | 0 | CH₂CH₂OMe |
| 3-83 | I | 1 | CH₂CH₂OMe |
| 3-84 | I | 2 | CH₂CH₂OMe |

TABLE 4
Compounds of the general formula (I) according to the
invention in which Q represents Q2 and R^{x} represents
a methyl group and R^{a} represents hydrogen

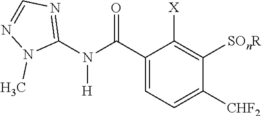

| No. | X | n | R |
|---|---|---|---|
| 4-1 | Me | 0 | Me |
| 4-2 | Me | 1 | Me |
| 4-3 | Me | 2 | Me |
| 4-4 | Me | 0 | Et |
| 4-5 | Me | 1 | Et |
| 4-6 | Me | 2 | Et |
| 4-7 | Me | 0 | CH₂c-Pr |
| 4-8 | Me | 1 | CH₂c-Pr |
| 4-9 | Me | 2 | CH₂c-Pr |
| 4-10 | Me | 0 | CH₂CH₂OMe |
| 4-11 | Me | 1 | CH₂CH₂OMe |
| 4-12 | Me | 2 | CH₂CH₂OMe |
| 4-13 | Et | 0 | Me |
| 4-14 | Et | 1 | Me |
| 4-15 | Et | 2 | Me |
| 4-16 | Et | 0 | Et |
| 4-17 | Et | 1 | Et |
| 4-18 | Et | 2 | Et |
| 4-19 | Et | 0 | CH₂c-Pr |
| 4-20 | Et | 1 | CH₂c-Pr |
| 4-21 | Et | 2 | CH₂c-Pr |
| 4-22 | Et | 0 | CH₂CH₂OMe |
| 4-23 | Et | 1 | CH₂CH₂OMe |
| 4-24 | Et | 2 | CH₂CH₂OMe |
| 4-25 | c-Pr | 0 | Me |
| 4-26 | c-Pr | 1 | Me |
| 4-27 | c-Pr | 2 | Me |
| 4-28 | c-Pr | 0 | Et |
| 4-29 | c-Pr | 1 | Et |
| 4-30 | c-Pr | 2 | Et |
| 4-31 | c-Pr | 0 | CH₂c-Pr |
| 4-32 | c-Pr | 1 | CH₂c-Pr |
| 4-33 | c-Pr | 2 | CH₂c-Pr |
| 4-34 | c-Pr | 0 | CH₂CH₂OMe |
| 4-35 | c-Pr | 1 | CH₂CH₂OMe |
| 4-36 | c-Pr | 2 | CH₂CH₂OMe |
| 4-37 | F | 0 | Me |
| 4-38 | F | 1 | Me |
| 4-39 | F | 2 | Me |
| 4-40 | F | 0 | Et |
| 4-41 | F | 1 | Et |
| 4-42 | F | 2 | Et |
| 4-43 | F | 0 | CH₂c-Pr |
| 4-44 | F | 1 | CH₂c-Pr |
| 4-45 | F | 2 | CH₂c-Pr |
| 4-46 | F | 0 | CH₂CH₂OMe |
| 4-47 | F | 1 | CH₂CH₂OMe |
| 4-48 | F | 2 | CH₂CH₂OMe |
| 4-49 | Cl | 0 | Me |
| 4-50 | Cl | 1 | Me |
| 4-51 | Cl | 2 | Me |
| 4-52 | Cl | 0 | Et |
| 4-53 | Cl | 1 | Et |
| 4-54 | Cl | 2 | Et |
| 4-55 | Cl | 0 | CH₂c-Pr |
| 4-56 | Cl | 1 | CH₂c-Pr |
| 4-57 | Cl | 2 | CH₂c-Pr |
| 4-58 | Cl | 0 | CH₂CH₂OMe |
| 4-59 | Cl | 1 | CH₂CH₂OMe |
| 4-60 | Cl | 2 | CH₂CH₂OMe |
| 4-61 | Br | 0 | Me |
| 4-62 | Br | 1 | Me |
| 4-63 | Br | 2 | Me |
| 4-64 | Br | 0 | Et |
| 4-65 | Br | 1 | Et |
| 4-66 | Br | 2 | Et |
| 4-67 | Br | 0 | CH₂c-Pr |
| 4-68 | Br | 1 | CH₂c-Pr |
| 4-69 | Br | 2 | CH₂c-Pr |
| 4-70 | Br | 0 | CH₂CH₂OMe |
| 4-71 | Br | 1 | CH₂CH₂OMe |
| 4-72 | Br | 2 | CH₂CH₂OMe |
| 4-73 | I | 0 | Me |
| 4-74 | I | 1 | Me |
| 4-75 | I | 2 | Me |
| 4-76 | I | 0 | Et |
| 4-77 | I | 1 | Et |
| 4-78 | I | 2 | Et |
| 4-79 | I | 0 | CH₂c-Pr |
| 4-80 | I | 1 | CH₂c-Pr |
| 4-81 | I | 2 | CH₂c-Pr |
| 4-82 | I | 0 | CH₂CH₂OMe |
| 4-83 | I | 1 | CH₂CH₂OMe |
| 4-84 | I | 2 | CH₂CH₂OMe |

TABLE 5
Compounds of the general formula (I) according to the
invention in which Q represents Q3 and R^{y} represents
a methyl group and R^{a} represents hydrogen

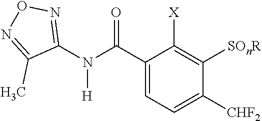

| No. | X | n | R |
|---|---|---|---|
| 5-1 | Me | 0 | Me |
| 5-2 | Me | 1 | Me |
| 5-3 | Me | 2 | Me |
| 5-4 | Me | 0 | Et |
| 5-5 | Me | 1 | Et |
| 5-6 | Me | 2 | Et |
| 5-7 | Me | 0 | CH₂c-Pr |
| 5-8 | Me | 1 | CH₂c-Pr |
| 5-9 | Me | 2 | CH₂c-Pr |
| 5-10 | Me | 0 | CH₂CH₂OMe |
| 5-11 | Me | 1 | CH₂CH₂OMe |
| 5-12 | Me | 2 | CH₂CH₂OMe |
| 5-13 | Et | 0 | Me |
| 5-14 | Et | 1 | Me |
| 5-15 | Et | 2 | Me |
| 5-16 | Et | 0 | Et |
| 5-17 | Et | 1 | Et |
| 5-18 | Et | 2 | Et |
| 5-19 | Et | 0 | CH₂c-Pr |
| 5-20 | Et | 1 | CH₂c-Pr |
| 5-21 | Et | 2 | CH₂c-Pr |
| 5-22 | Et | 0 | CH₂CH₂OMe |
| 5-23 | Et | 1 | CH₂CH₂OMe |
| 5-24 | Et | 2 | CH₂CH₂OMe |
| 5-25 | c-Pr | 0 | Me |
| 5-26 | c-Pr | 1 | Me |
| 5-27 | c-Pr | 2 | Me |
| 5-28 | c-Pr | 0 | Et |
| 5-29 | c-Pr | 1 | Et |
| 5-30 | c-Pr | 2 | Et |
| 5-31 | c-Pr | 0 | CH₂c-Pr |
| 5-32 | c-Pr | 1 | CH₂c-Pr |
| 5-33 | c-Pr | 2 | CH₂c-Pr |
| 5-34 | c-Pr | 0 | CH₂CH₂OMe |
| 5-35 | c-Pr | 1 | CH₂CH₂OMe |
| 5-36 | c-Pr | 2 | CH₂CH₂OMe |
| 5-37 | F | 0 | Me |
| 5-38 | F | 1 | Me |
| 5-39 | F | 2 | Me |
| 5-40 | F | 0 | Et |
| 5-41 | F | 1 | Et |
| 5-42 | F | 2 | Et |
| 5-43 | F | 0 | CH₂c-Pr |
| 5-44 | F | 1 | CH₂c-Pr |
| 5-45 | F | 2 | CH₂c-Pr |
| 5-46 | F | 0 | CH₂CH₂OMe |
| 5-47 | F | 1 | CH₂CH₂OMe |
| 5-48 | F | 2 | CH₂CH₂OMe |
| 5-49 | Cl | 0 | Me |
| 5-50 | Cl | 1 | Me |
| 5-51 | Cl | 2 | Me |
| 5-52 | Cl | 0 | Et |
| 5-53 | Cl | 1 | Et |
| 5-54 | Cl | 2 | Et |
| 5-55 | Cl | 0 | CH₂c-Pr |
| 5-56 | Cl | 1 | CH₂c-Pr |
| 5-57 | Cl | 2 | CH₂c-Pr |
| 5-58 | Cl | 0 | CH₂CH₂OMe |
| 5-59 | Cl | 1 | CH₂CH₂OMe |
| 5-60 | Cl | 2 | CH₂CH₂OMe |
| 5-61 | Br | 0 | Me |
| 5-62 | Br | 1 | Me |
| 5-63 | Br | 2 | Me |
| 5-64 | Br | 0 | Et |
| 5-65 | Br | 1 | Et |
| 5-66 | Br | 2 | Et |
| 5-67 | Br | 0 | CH₂c-Pr |
| 5-68 | Br | 1 | CH₂c-Pr |
| 5-69 | Br | 2 | CH₂c-Pr |
| 5-70 | Br | 0 | CH₂CH₂OMe |
| 5-71 | Br | 1 | CH₂CH₂OMe |
| 5-72 | Br | 2 | CH₂CH₂OMe |
| 5-73 | I | 0 | Me |
| 5-74 | I | 1 | Me |
| 5-75 | I | 2 | Me |
| 5-76 | I | 0 | Et |
| 5-77 | I | 1 | Et |
| 5-78 | I | 2 | Et |
| 5-79 | I | 0 | CH₂c-Pr |
| 5-80 | I | 1 | CH₂c-Pr |
| 5-81 | I | 2 | CH₂c-Pr |
| 5-82 | I | 0 | CH₂CH₂OMe |
| 5-83 | I | 1 | CH₂CH₂OMe |
| 5-84 | I | 2 | CH₂CH₂OMe |

TABLE 6
Compounds of the general formula (I) according
to the invention in which Q represents Q3 and R^{y}
represents chlorine and R^{a} represents hydrogen

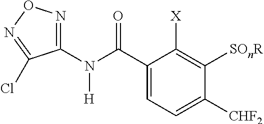

| No. | X | n | R |
|---|---|---|---|
| 6-1 | Me | 0 | Me |
| 6-2 | Me | 1 | Me |
| 6-3 | Me | 2 | Me |
| 6-4 | Me | 0 | Et |
| 6-5 | Me | 1 | Et |
| 6-6 | Me | 2 | Et |
| 6-7 | Me | 0 | CH₂c-Pr |
| 6-8 | Me | 1 | CH₂c-Pr |
| 6-9 | Me | 2 | CH₂c-Pr |
| 6-10 | Me | 0 | CH₂CH₂OMe |
| 6-11 | Me | 1 | CH₂CH₂OMe |
| 6-12 | Me | 2 | CH₂CH₂OMe |
| 6-13 | Et | 0 | Me |
| 6-14 | Et | 1 | Me |
| 6-15 | Et | 2 | Me |
| 6-16 | Et | 0 | Et |
| 6-17 | Et | 1 | Et |
| 6-18 | Et | 2 | Et |
| 6-19 | Et | 0 | CH₂c-Pr |
| 6-20 | Et | 1 | CH₂c-Pr |
| 6-21 | Et | 2 | CH₂c-Pr |
| 6-22 | Et | 0 | CH₂CH₂OMe |
| 6-23 | Et | 1 | CH₂CH₂OMe |
| 6-24 | Et | 2 | CH₂CH₂OMe |
| 6-25 | c-Pr | 0 | Me |
| 6-26 | c-Pr | 1 | Me |
| 6-27 | c-Pr | 2 | Me |
| 6-28 | c-Pr | 0 | Et |
| 6-29 | c-Pr | 1 | Et |
| 6-30 | c-Pr | 2 | Et |
| 6-31 | c-Pr | 0 | CH₂c-Pr |
| 6-32 | c-Pr | 1 | CH₂c-Pr |
| 6-33 | c-Pr | 2 | CH₂c-Pr |
| 6-34 | c-Pr | 0 | CH₂CH₂OMe |
| 6-35 | c-Pr | 1 | CH₂CH₂OMe |
| 6-36 | c-Pr | 2 | CH₂CH₂OMe |
| 6-37 | F | 0 | Me |
| 6-38 | F | 1 | Me |
| 6-39 | F | 2 | Me |
| 6-40 | F | 0 | Et |
| 6-41 | F | 1 | Et |
| 6-42 | F | 2 | Et |
| 6-43 | F | 0 | CH₂c-Pr |
| 6-44 | F | 1 | CH₂c-Pr |
| 6-45 | F | 2 | CH₂c-Pr |
| 6-46 | F | 0 | CH₂CH₂OMe |
| 6-47 | F | 1 | CH₂CH₂OMe |
| 6-48 | F | 2 | CH₂CH₂OMe |
| 6-49 | Cl | 0 | Me |
| 6-50 | Cl | 1 | Me |
| 6-51 | Cl | 2 | Me |
| 6-52 | Cl | 0 | Et |
| 6-53 | Cl | 1 | Et |
| 6-54 | Cl | 2 | Et |
| 6-55 | Cl | 0 | CH₂c-Pr |
| 6-56 | Cl | 1 | CH₂c-Pr |
| 6-57 | Cl | 2 | CH₂c-Pr |
| 6-58 | Cl | 0 | CH₂CH₂OMe |
| 6-59 | Cl | 1 | CH₂CH₂OMe |
| 6-60 | Cl | 2 | CH₂CH₂OMe |
| 6-61 | Br | 0 | Me |
| 6-62 | Br | 1 | Me |
| 6-63 | Br | 2 | Me |
| 6-64 | Br | 0 | Et |
| 6-65 | Br | 1 | Et |
| 6-66 | Br | 2 | Et |
| 6-67 | Br | 0 | CH₂c-Pr |
| 6-68 | Br | 1 | CH₂c-Pr |
| 6-69 | Br | 2 | CH₂c-Pr |
| 6-70 | Br | 0 | CH₂CH₂OMe |
| 6-71 | Br | 1 | CH₂CH₂OMe |
| 6-72 | Br | 2 | CH₂CH₂OMe |
| 6-73 | I | 0 | Me |
| 6-74 | I | 1 | Me |
| 6-75 | I | 2 | Me |
| 6-76 | I | 0 | Et |
| 6-77 | I | 1 | Et |
| 6-78 | I | 2 | Et |
| 6-79 | I | 0 | CH₂c-Pr |
| 6-80 | I | 1 | CH₂c-Pr |
| 6-81 | I | 2 | CH₂c-Pr |
| 6-82 | I | 0 | CH₂CH₂OMe |
| 6-83 | I | 1 | CH₂CH₂OMe |
| 6-84 | I | 2 | CH₂CH₂OMe |

TABLE 7
Compounds of the general formula (I) according
to the invention in which Q represents Q4 and R^{z}
represents a methyl group and R^{a} represents hydrogen

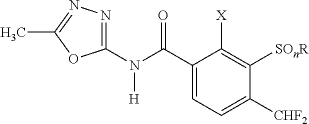

| No. | X | n | R |
|---|---|---|---|
| 7-1 | Me | 0 | Me |
| 7-2 | Me | 1 | Me |
| 7-3 | Me | 2 | Me |
| 7-4 | Me | 0 | Et |
| 7-5 | Me | 0 | CH₂c-Pr |
| 7-6 | Me | 1 | CH₂c-Pr |
| 7-7 | Me | 2 | CH₂c-Pr |
| 7-8 | Me | 0 | CH₂CH₂OMe |
| 7-9 | Me | 1 | CH₂CH₂OMe |
| 7-10 | Me | 2 | CH₂CH₂OMe |
| 7-11 | Et | 0 | Me |
| 7-12 | Et | 1 | Me |
| 7-13 | Et | 2 | Me |
| 7-14 | Et | 0 | Et |
| 7-15 | Et | 1 | Et |
| 7-16 | Et | 2 | Et |
| 7-17 | Et | 0 | CH₂c-Pr |
| 7-18 | Et | 1 | CH₂c-Pr |
| 7-19 | Et | 2 | CH₂c-Pr |
| 7-20 | Et | 0 | CH₂CH₂OMe |
| 7-21 | Et | 1 | CH₂CH₂OMe |
| 7-22 | Et | 2 | CH₂CH₂OMe |
| 7-23 | c-Pr | 0 | Me |
| 7-24 | c-Pr | 1 | Me |
| 7-25 | c-Pr | 2 | Me |
| 7-26 | c-Pr | 0 | Et |
| 7-27 | c-Pr | 1 | Et |
| 7-28 | c-Pr | 2 | Et |
| 7-29 | c-Pr | 0 | CH₂c-Pr |
| 7-30 | c-Pr | 1 | CH₂c-Pr |
| 7-31 | c-Pr | 2 | CH₂c-Pr |
| 7-32 | c-Pr | 0 | CH₂CH₂OMe |
| 7-33 | c-Pr | 1 | CH₂CH₂OMe |
| 7-34 | c-Pr | 2 | CH₂CH₂OMe |
| 7-35 | F | 0 | Me |
| 7-36 | F | 1 | Me |
| 7-37 | F | 2 | Me |
| 7-38 | F | 0 | Et |
| 7-39 | F | 1 | Et |
| 7-40 | F | 2 | Et |
| 7-41 | F | 0 | CH₂c-Pr |
| 7-42 | F | 1 | CH₂c-Pr |
| 7-43 | F | 2 | CH₂c-Pr |
| 7-44 | F | 0 | CH₂CH₂OMe |
| 7-45 | F | 1 | CH₂CH₂OMe |
| 7-46 | F | 2 | CH₂CH₂OMe |
| 7-47 | Cl | 0 | Me |
| 7-48 | Cl | 1 | Me |
| 7-49 | Cl | 2 | Me |
| 7-50 | Cl | 0 | Et |
| 7-51 | Cl | 1 | Et |
| 7-52 | Cl | 2 | Et |
| 7-53 | Cl | 0 | CH₂c-Pr |
| 7-54 | Cl | 1 | CH₂c-Pr |
| 7-55 | Cl | 2 | CH₂c-Pr |
| 7-56 | Cl | 0 | CH₂CH₂OMe |
| 7-57 | Cl | 1 | CH₂CH₂OMe |
| 7-58 | Cl | 2 | CH₂CH₂OMe |
| 7-59 | Br | 0 | Me |
| 7-60 | Br | 1 | Me |
| 7-61 | Br | 2 | Me |
| 7-62 | Br | 0 | Et |
| 7-63 | Br | 1 | Et |
| 7-64 | Br | 2 | Et |
| 7-65 | Br | 0 | CH₂c-Pr |
| 7-66 | Br | 1 | CH₂c-Pr |
| 7-67 | Br | 2 | CH₂c-Pr |
| 7-68 | Br | 0 | CH₂CH₂OMe |
| 7-69 | Br | 1 | CH₂CH₂OMe |
| 7-70 | Br | 2 | CH₂CH₂OMe |
| 7-71 | I | 0 | Me |
| 7-72 | I | 1 | Me |
| 7-73 | I | 2 | Me |
| 7-74 | I | 0 | Et |
| 7-75 | I | 1 | Et |
| 7-76 | I | 2 | Et |
| 7-77 | I | 0 | CH₂c-Pr |
| 7-78 | I | 1 | CH₂c-Pr |
| 7-79 | I | 2 | CH₂c-Pr |
| 7-80 | I | 0 | CH₂CH₂OMe |
| 7-81 | I | 1 | CH₂CH₂OMe |
| 7-82 | I | 2 | CH₂CH₂OMe |

TABLE 8
Inventive compounds of the general formula
(I) in the form of the sodium salts, in which
Q is Q1 and R^{x} is a methyl group

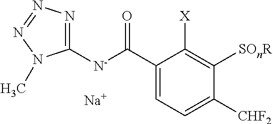

| No. | X | n | R |
|---|---|---|---|
| 8-1 | Me | 0 | Me |
| 8-2 | Me | 1 | Me |
| 8-3 | Me | 2 | Me |
| 8-4 | Me | 0 | Et |
| 8-5 | Me | 1 | Et |
| 8-6 | Me | 2 | Et |
| 8-7 | Me | 0 | CH₂c-Pr |
| 8-8 | Me | 1 | CH₂c-Pr |
| 8-9 | Me | 2 | CH₂c-Pr |
| 8-10 | Me | 0 | CH₂CH₂OMe |
| 8-11 | Me | 1 | CH₂CH₂OMe |
| 8-12 | Me | 2 | CH₂CH₂OMe |
| 8-13 | Et | 0 | Me |
| 8-14 | Et | 1 | Me |
| 8-15 | Et | 2 | Me |
| 8-16 | Et | 0 | Et |
| 8-17 | Et | 1 | Et |
| 8-18 | Et | 2 | Et |
| 8-19 | Et | 0 | CH₂c-Pr |
| 8-20 | Et | 1 | CH₂c-Pr |
| 8-21 | Et | 2 | CH₂c-Pr |
| 8-22 | Et | 0 | CH₂CH₂OMe |
| 8-23 | Et | 1 | CH₂CH₂OMe |
| 8-24 | Et | 2 | CH₂CH₂OMe |
| 8-25 | c-Pr | 0 | Me |
| 8-26 | c-Pr | 1 | Me |
| 8-27 | c-Pr | 2 | Me |
| 8-28 | c-Pr | 0 | Et |
| 8-29 | c-Pr | 1 | Et |
| 8-30 | c-Pr | 2 | Et |
| 8-31 | c-Pr | 0 | CH₂c-Pr |
| 8-32 | c-Pr | 1 | CH₂c-Pr |
| 8-33 | c-Pr | 2 | CH₂c-Pr |
| 8-34 | c-Pr | 0 | CH₂CH₂OMe |
| 8-35 | c-Pr | 1 | CH₂CH₂OMe |
| 8-36 | c-Pr | 2 | CH₂CH₂OMe |
| 8-37 | F | 0 | Me |
| 8-38 | F | 1 | Me |
| 8-39 | F | 2 | Me |
| 8-40 | F | 0 | Et |
| 8-41 | F | 1 | Et |
| 8-42 | F | 2 | Et |
| 8-43 | F | 0 | CH₂c-Pr |
| 8-44 | F | 1 | CH₂c-Pr |
| 8-45 | F | 2 | CH₂c-Pr |
| 8-46 | F | 0 | CH₂CH₂OMe |
| 8-47 | F | 1 | CH₂CH₂OMe |
| 8-48 | F | 2 | CH₂CH₂OMe |
| 8-49 | Cl | 0 | Me |
| 8-50 | Cl | 1 | Me |
| 8-51 | Cl | 2 | Me |
| 8-52 | Cl | 0 | Et |
| 8-53 | Cl | 1 | Et |
| 8-54 | Cl | 2 | Et |
| 8-55 | Cl | 0 | CH₂c-Pr |
| 8-56 | Cl | 1 | CH₂c-Pr |
| 8-57 | Cl | 2 | CH₂c-Pr |
| 8-58 | Cl | 0 | CH₂CH₂OMe |
| 8-59 | Cl | 1 | CH₂CH₂OMe |
| 8-60 | Cl | 2 | CH₂CH₂OMe |
| 8-61 | Br | 0 | Me |
| 8-62 | Br | 1 | Me |
| 8-63 | Br | 2 | Me |
| 8-64 | Br | 0 | Et |
| 8-65 | Br | 1 | Et |
| 8-66 | Br | 2 | Et |
| 8-67 | Br | 0 | CH₂c-Pr |
| 8-68 | Br | 1 | CH₂c-Pr |
| 8-69 | Br | 2 | CH₂c-Pr |
| 8-70 | Br | 0 | CH₂CH₂OMe |
| 8-71 | Br | 1 | CH₂CH₂OMe |
| 8-72 | Br | 2 | CH₂CH₂OMe |
| 8-73 | I | 0 | Me |
| 8-74 | I | 1 | Me |
| 8-75 | I | 2 | Me |
| 8-76 | I | 0 | Et |
| 8-77 | I | 1 | Et |
| 8-78 | I | 2 | Et |
| 8-79 | I | 0 | CH₂c-Pr |
| 8-80 | I | 1 | CH₂c-Pr |
| 8-81 | I | 2 | CH₂c-Pr |
| 8-82 | I | 0 | CH₂CH₂OMe |
| 8-83 | I | 1 | CH₂CH₂OMe |
| 8-84 | I | 2 | CH₂CH₂OMe |

TABLE 9
Compounds of the general formula
(II) according to the invention

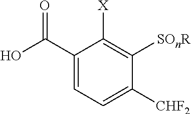

| No. | X | n | R |
|---|---|---|---|
| 9-1 | c-Pr | 0 | Me |
| 9-2 | c-Pr | 1 | Me |
| 9-3 | c-Pr | 2 | Me |
| 9-4 | c-Pr | 0 | Et |
| 9-5 | c-Pr | 1 | Et |
| 9-6 | c-Pr | 2 | Et |
| 9-7 | c-Pr | 0 | CH₂c-Pr |
| 9-8 | c-Pr | 1 | CH₂c-Pr |
| 9-9 | c-Pr | 2 | CH₂c-Pr |
| 9-10 | c-Pr | 0 | CH₂CH₂OMe |
| 9-11 | c-Pr | 1 | CH₂CH₂OMe |
| 9-12 | c-Pr | 2 | CH₂CH₂OMe |
| 9-13 | F | 0 | Me |
| 9-14 | F | 1 | Me |
| 9-15 | F | 2 | Me |
| 9-16 | F | 0 | Et |
| 9-17 | F | 1 | Et |
| 9-18 | F | 2 | Et |
| 9-19 | F | 0 | CH₂c-Pr |
| 9-20 | F | 1 | CH₂c-Pr |
| 9-21 | F | 2 | CH₂c-Pr |
| 9-22 | F | 0 | CH₂CH₂OMe |
| 9-23 | F | 1 | CH₂CH₂OMe |
| 9-24 | F | 2 | CH₂CH₂OMe |
| 9-25 | Cl | 0 | Me |
| 9-26 | Cl | 1 | Me |
| 9-27 | Cl | 2 | Me |
| 9-28 | Cl | 0 | Et |
| 9-29 | Cl | 1 | Et |
| 9-30 | Cl | 2 | Et |
| 9-31 | Cl | 0 | CH₂c-Pr |
| 9-32 | Cl | 1 | CH₂c-Pr |
| 9-33 | Cl | 2 | CH₂c-Pr |
| 9-34 | Cl | 0 | CH₂CH₂OMe |
| 9-35 | Cl | 1 | CH₂CH₂OMe |
| 9-36 | Cl | 2 | CH₂CH₂OMe |
| 9-37 | Br | 0 | Me |
| 9-38 | Br | 1 | Me |
| 9-39 | Br | 2 | Me |
| 9-40 | Br | 0 | Et |
| 9-41 | Br | 1 | Et |
| 9-42 | Br | 2 | Et |
| 9-43 | Br | 0 | CH₂c-Pr |
| 9-44 | Br | 1 | CH₂c-Pr |
| 9-45 | Br | 2 | CH₂c-Pr |
| 9-46 | Br | 0 | CH₂CH₂OMe |
| 9-47 | Br | 1 | CH₂CH₂OMe |
| 9-48 | Br | 2 | CH₂CH₂OMe |
| 9-49 | I | 0 | Me |
| 9-50 | I | 1 | Me |
| 9-51 | I | 2 | Me |
| 9-52 | I | 0 | Et |
| 9-53 | I | 1 | Et |
| 9-54 | I | 2 | Et |
| 9-55 | I | 0 | CH₂c-Pr |
| 9-56 | I | 1 | CH₂c-Pr |
| 9-57 | I | 2 | CH₂c-Pr |
| 9-58 | I | 0 | CH₂CH₂OMe |
| 9-59 | I | 1 | CH₂CH₂OMe |
| 9-60 | I | 2 | CH₂CH₂OMe |

TABLE 10
Compounds of the general formula
(III) according to the invention

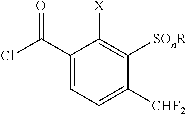

| No. | X | n | R |
|---|---|---|---|
| 10-1 | Me | 0 | Me |
| 10-2 | Me | 1 | Me |
| 10-3 | Me | 2 | Me |
| 10-4 | Me | 0 | Et |
| 10-5 | Me | 1 | Et |
| 10-6 | Me | 2 | Et |
| 10-7 | Me | 0 | CH₂c-Pr |
| 10-8 | Me | 1 | CH₂c-Pr |
| 10-9 | Me | 2 | CH₂c-Pr |
| 10-10 | Me | 0 | CH₂CH₂OMe |
| 10-11 | Me | 1 | CH₂CH₂OMe |
| 10-12 | Me | 2 | CH₂CH₂OMe |
| 10-13 | Et | 0 | Me |
| 10-14 | Et | 1 | Me |
| 10-15 | Et | 2 | Me |
| 10-16 | Et | 0 | Et |
| 10-17 | Et | 1 | Et |
| 10-18 | Et | 2 | Et |
| 10-19 | Et | 0 | CH₂c-Pr |
| 10-20 | Et | 1 | CH₂c-Pr |
| 10-21 | Et | 2 | CH₂c-Pr |
| 10-22 | Et | 0 | CH₂CH₂OMe |
| 10-23 | Et | 1 | CH₂CH₂OMe |
| 10-24 | Et | 2 | CH₂CH₂OMe |
| 10-25 | c-Pr | 0 | Me |
| 10-26 | c-Pr | 1 | Me |
| 10-27 | c-Pr | 2 | Me |
| 10-28 | c-Pr | 0 | Et |
| 10-29 | c-Pr | 1 | Et |
| 10-30 | c-Pr | 2 | Et |
| 10-31 | c-Pr | 0 | CH₂c-Pr |
| 10-32 | c-Pr | 1 | CH₂c-Pr |
| 10-33 | c-Pr | 2 | CH₂c-Pr |
| 10-34 | c-Pr | 0 | CH₂CH₂OMe |
| 10-35 | c-Pr | 1 | CH₂CH₂OMe |
| 10-36 | c-Pr | 2 | CH₂CH₂OMe |
| 10-37 | F | 0 | Me |
| 10-38 | F | 1 | Me |
| 10-39 | F | 2 | Me |
| 10-40 | F | 0 | Et |
| 10-41 | F | 1 | Et |
| 10-42 | F | 2 | Et |
| 10-43 | F | 0 | CH₂c-Pr |
| 10-44 | F | 1 | CH₂c-Pr |
| 10-45 | F | 2 | CH₂c-Pr |
| 10-46 | F | 0 | CH₂CH₂OMe |
| 10-47 | F | 1 | CH₂CH₂OMe |
| 10-48 | F | 2 | CH₂CH₂OMe |
| 10-49 | Cl | 0 | Me |
| 10-50 | Cl | 1 | Me |
| 10-51 | Cl | 2 | Me |
| 10-52 | Cl | 0 | Et |
| 10-53 | Cl | 1 | Et |
| 10-54 | Cl | 2 | Et |
| 10-55 | Cl | 0 | CH₂c-Pr |
| 10-56 | Cl | 1 | CH₂c-Pr |
| 10-57 | Cl | 2 | CH₂c-Pr |
| 10-58 | Cl | 0 | CH₂CH₂OMe |
| 10-59 | Cl | 1 | CH₂CH₂OMe |
| 10-60 | Cl | 2 | CH₂CH₂OMe |
| 10-61 | Br | 0 | Me |
| 10-62 | Br | 1 | Me |
| 10-63 | Br | 2 | Me |
| 10-64 | Br | 0 | Et |
| 10-65 | Br | 1 | Et |
| 10-66 | Br | 2 | Et |
| 10-67 | Br | 0 | CH₂c-Pr |
| 10-68 | Br | 1 | CH₂c-Pr |
| 10-69 | Br | 2 | CH₂c-Pr |
| 10-70 | Br | 0 | CH₂CH₂OMe |
| 10-71 | Br | 1 | CH₂CH₂OMe |
| 10-72 | Br | 2 | CH₂CH₂OMe |
| 10-73 | I | 0 | Me |
| 10-74 | I | 1 | Me |
| 10-75 | I | 2 | Me |
| 10-76 | I | 0 | Et |
| 10-77 | I | 1 | Et |
| 10-78 | I | 2 | Et |
| 10-79 | I | 0 | CH₂c-Pr |
| 10-80 | I | 1 | CH₂c-Pr |
| 10-81 | I | 2 | CH₂c-Pr |
| 10-82 | I | 0 | CH₂CH₂OMe |
| 10-83 | I | 1 | CH₂CH₂OMe |
| 10-84 | I | 2 | CH₂CH₂OMe |

NMR data for numerous compounds of the formula (I) according to the invention mentioned in the tables above are disclosed below using the NMR peak list method. Here, the ¹H NMR data of selected examples are stated in the form of ¹H NMR peak lists. For each signal peak, first the δ value in ppm and then the signal intensity in round brackets are listed. The δ value—signal intensity number pairs for different signal peaks are listed with separation from one another by semicolons. The peak list for one example therefore takes the form of:

δ₁ (intensity₁); δ₂ (intensity₂); . . . ; δᵢ (intensityᵢ); . . . ; δₙ (intensityₙ)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum. The lists of the ¹H NMR peaks are similar to the conventional ¹H NMR printouts and thus usually contain all peaks listed in a conventional NMR interpretation. In addition, like conventional ¹H NMR printouts, they may show solvent signals, signals of stereoisomers of the target compounds which are likewise provided by the invention, and/or peaks of impurities.

In the reporting of compound signals within the delta range of solvents and/or water, our lists of ¹H NMR peaks show the standard solvent peaks, for example peaks of DMSO in DMSO-D₆ and the peak of water, which usually have a high intensity on average.

The peaks of stereoisomers of the compounds of the invention and/or peaks of impurities usually have a lower intensity on average than the peaks of the compounds of the invention (for example with a purity of >90%).

Such stereoisomers and/or impurities may be typical of the particular preparation process. Their peaks can thus help in identifying reproduction of our preparation process with reference to “by-product fingerprints”.

An expert calculating the peaks of the target compounds by known methods (MestreC, ACD simulation, but also with empirically evaluated expected values) can, if required, isolate the peaks of the compounds of the invention, optionally using additional intensity filters. This isolation would be similar to the peak picking in question in conventional ¹H NMR interpretation.

Example 1-1: ¹H-NMR (400.0 MHz, CDCl3): δ =
7.79 (0.78); 7.77 (1.41); 7.723 (1.33); 7.703 (0.72); 7.518 (0.54); 7.506 (1.01); 7.368 (2.17); 7.26 (92.56); 7.229 (1.07); 6.996 (0.51); 4.127 (14.78);
2.797 (9.61); 2.308 (16); 2.252 (0.56); 1.553 (1.29); 0.008 (1.14); 0 (32.43); −0.008 (0.97)
Example 1-2: ¹H-NMR (400.0 MHz, d₆-DMSO): δ =
11.755 (0.94); 7.995 (0.74); 7.923 (0.57); 7.902 (0.93); 7.842 (1.16); 7.821 (0.68); 4.007 (16); 3.311 (53.71); 3.002 (12.55); 2.674 (0.69); 2.669
(0.92); 2.665 (0.7); 2.59 (7.06); 2.523 (2.83); 2.518 (4.26); 2.51 (55.71); 2.505 (120.68); 2.5 (167.23); 2.496 (116.4); 2.491 (51.7); 2.45 (0.53);
2.332 (0.71); 2.327 (0.96); 2.323 (0.69); 0.008 (0.95); 0 (31.89); −0.008 (0.89)
Example 1-3: ¹H-NMR (400.0 MHz, d₆-DMSO): δ =
11.829 (0.84); 8.047 (0.55); 7.937 (0.88); 7.919 (0.94); 7.783 (1.21); 7.646 (0.6); 4.021 (16); 3.446 (10.93); 3.309 (31.28); 2.75 (7.59); 2.669
(0.55); 2.523 (1.58); 2.518 (2.4); 2.509 (30.08); 2.505 (65.21); 2.5 (91.14); 2.496 (63.77); 2.491 (28.46); 2.327 (0.53); 0.008 (0.54); 0 (17.36)
Example 1-4: ¹H-NMR (400.0 MHz, CDCl3): δ =
7.778 (0.71); 7.761 (1.15); 7.715 (1.06); 7.523 (1.62); 7.519 (1.13); 7.385 (3.47); 7.27 (0.65); 7.27 (0.73); 7.269 (0.81); 7.268 (0.84); 7.267 (1.06);
7.266 (1.35); 7.26 (155.87); 7.247 (2); 6.996 (0.85); 4.128 (1.1); 4.117 (16); 2.784 (2.73); 2.773 (8.76); 2.766 (7.1); 2.747 (5.05); 2.728 (1.75);
2.042 (1.82); 1.57 (2.2); 1.284 (0.63); 1.275 (1.24); 1.265 (1.7); 1.257 (2.02); 1.239 (1.19); 1.234 (7.07); 1.216 (14.14); 1.197 (6.63); 0.899
(0.91); 0.882 (3.32); 0.864 (1.23); 0.008 (1.65); 0.006 (0.61); 0.006 (0.65); 0.005 (0.77); 0 (55.26); −0.006 (0.8); −0.007 (0.64); −0.008 (1.72)
Example 1-5: ¹H-NMR (400.0 MHz, d₆-DMSO): δ =
11.757 (1); 7.932 (0.55); 7.912 (0.92); 7.856 (1.09); 7.836 (0.68); 5.753 (0.57); 4.008 (16); 3.31 (44.61); 3.227 (0.59); 3.208 (0.58); 3.126 (0.66);
3.107 (0.69); 2.568 (5.11); 2.523 (1.11); 2.518 (1.62); 2.509 (21.94); 2.505 (46.65); 2.5 (64.18); 2.496 (44.74); 2.491 (20.18); 1.3 (2.55); 1.282
(5.31); 1.263 (2.39); 0 (4.67)
Example 1-6: ¹H-NMR (400.0 MHz, d₆-DMSO): δ =
11.826 (0.98); 8.065 (0.55); 7.964 (0.9); 7.944 (0.68); 7.914 (0.63); 7.778 (1.36); 7.641 (0.65); 4.022 (16); 3.542 (0.71); 3.524 (2.42); 3.506 (2.48);
3.488 (0.75); 3.309 (87.5); 2.742 (8.48); 2.669 (0.56); 2.523 (1.92); 2.509 (34.37); 2.505 (72.77); 2.5 (99.75); 2.496 (69.83); 2.491 (31.46);
2.327 (0.57); 1.267 (2.62); 1.248 (5.94); 1.23 (2.62); 0 (3.28)
Example 1-13: ¹H-NMR (400.0 MHz, CDCl3): δ =
7.763 (0.8); 7.743 (1.83); 7.713 (2.2); 7.693 (0.97); 7.524 (0.88); 7.386 (1.93); 7.267 (13.77); 7.248 (0.97); 4.104 (15.81); 3.235 (0.68); 3.216
(2.31); 3.198 (2.35); 3.179 (0.72); 2.331 (16); 1.27 (2.84); 1.252 (6.76); 1.233 (2.81); 0 (5.47)
Example 1-14: ¹H-NMR (400.0 MHz, d₆-DMSO):
δ =
11.796 (1.2); 8.121 (0.6); 7.962 (0.5); 7.942 (0.9); 7.892 (1.2); 7.871 (0.7); 4.010 (16.0); 3.310 (34.6); 3.044 (11.9); 3.032 (0.5); 2.523 (1.2); 2.518
(1.8); 2.509 (22.8); 2.505 (49.0); 2.500 (68.3); 2.496 (47.6); 2.491 (21.1); 1.242 (1.8); 1.223 (4.3); 1.205 (1.7); 0.008 (0.7); 0.000 (23.5); −0.009
(0.7)
Example 1-15: ¹H-NMR (400.0 MHz, d₆-DMSO):
δ =
11.890 (0.7); 7.958 (0.8); 7.938 (0.7); 7.777 (1.2); 7.641 (0.6); 4.026 (16.0); 3.440 (10.7); 3.310 (22.0); 3.226 (1.1); 3.208 (1.1); 2.518 (0.8); 2.510
(12.2); 2.505 (27.2); 2.500 (38.4); 2.496 (26.7); 2.491 (11.7); 1.281 (1.5); 1.263 (4.0); 1.244 (1.6); 0.000 (17.4)
Example 1-16: ¹H-NMR (400.0 MHz, CDCl3): δ =
7.792 (0.77); 7.772 (1.59); 7.735 (2.01); 7.715 (0.98); 7.537 (0.8); 7.399 (1.81); 7.261 (16.53); 5.298 (1.09); 4.119 (16); 3.253 (0.59); 3.234 (2.01);
3.216 (2.06); 3.197 (0.64); 2.797 (1.14); 2.778 (3.65); 2.76 (3.69); 2.741 (1.19); 1.258 (4.01); 1.239 (8.35); 1.228 (2.65); 1.221 (4.02); 1.21
(6.13); 1.191 (2.52); 0 (6.53)
Example 1-17: ¹H-NMR (400.0 MHz, d₆-DMSO): δ =
11.805 (1.75); 7.967 (0.92); 7.947 (1.25); 7.895 (1.45); 7.875 (0.9); 4.01 (16); 3.31 (32.77); 3.286 (0.72); 3.268 (0.74); 3.253 (0.9); 3.235 (0.82);
3.112 (0.68); 3.093 (0.81); 3.078 (0.61); 3.06 (0.59); 2.889 (0.52); 2.669 (0.51); 2.523 (0.75); 2.518 (1.23); 2.509 (21.71); 2.505 (54.6); 2.5
(86.23); 2.496 (78.63); 2.491 (50.39); 2.45 (0.77); 2.327 (0.57); 1.333 (2.67); 1.314 (5.76); 1.295 (2.73); 1.236 (2.18); 1.218 (5.11); 1.199 (2.25);
0.008 (0.55); 0 (20.1)
Example 1-18: ¹H-NMR (400.0 MHz, d₆-DMSO): δ =
11.893 (0.71); 7.984 (0.74); 7.963 (0.58); 7.907 (0.53); 7.771 (1.23); 7.634 (0.63); 4.026 (16); 3.512 (0.62); 3.494 (2.16); 3.475 (2.18); 3.457
(0.67); 3.31 (42.94); 3.208 (1); 3.19 (0.97); 2.523 (0.75); 2.518 (1.2); 2.51 (21.1); 2.505 (47.16); 2.5 (66.67); 2.496 (46.4); 2.491 (20.66); 2.45
(0.51); 1.276 (2.41); 1.267 (1.83); 1.257 (5.73); 1.249 (4.46); 1.239 (2.59); 1.231 (1.78); 0.008 (0.64); 0 (25.34); −0.009 (0.75)
Example 1-25: ¹H-NMR (400.0 MHz, d₆-DMSO):
δ = 11.6077 (1.3); 7.6627 (2.3); 7.6435 (0.5); 7.5981 (0.9); 7.4606 (2.0); 7.3232 (0.9); 4.0382 (16.0); 3.3110 (39.5); 2.5224 (1.2);
2.5048 (39.0); 2.5004 (50.9); 2.4959 (37.0); 2.4915 (18.2); 2.4274 (8.2); 1.1329 (0.5); 1.1211 (1.7); 1.1177 (1.7); 1.0998 (1.7);
1.0850 (0.6); 0.6649 (0.6); 0.6508 (2.0); 0.6390 (2.0); 0.6247 (0.5); −0.0002 (8.8)
Example 1-26: ¹H-NMR (400.0 MHz, d₆-DMSO):
δ = 8.4191 (0.9); 8.2814 (2.0); 8.1437 (1.0); 7.9077 (1.1); 7.8874 (1.8); 7.8205 (2.0); 7.8004 (1.3); 4.0383 (1.3); 4.0204 (1.6);
4.0088 (14.3); 3.3113 (10.0); 3.0436 (16.0); 2.5229 (1.6); 2.5182 (2.2); 2.5095 (22.6); 2.5050 (46.2); 2.5005 (62.8); 2.4959
(44.0); 2.4914 (20.3); 2.0763 (0.7); 1.9878 (5.4); 1.2587 (0.6); 1.2366 (2.5); 1.1923 (1.5); 1.1745 (3.0); 1.1567 (1.7); 1.1286
(0.8); 1.1188 (0.7); 1.0038 (0.6); 0.9920 (0.7); 0.8539 (0.5); 0.7331 (0.6); 0.7162 (1.5); 0.7078 (1.3); 0.7015 (1.4); 0.6834 (0.6); −0.0002
(12.8)
Example 1-27: ¹H-NMR (400.0 MHz, d₆-DMSO):
δ = 11.7346 (0.8); 7.9246 (0.7); 7.8507 (0.5); 7.7145 (1.3); 7.5784 (0.6); 4.0346 (16.0); 3.5507 (11.0); 3.3125 (58.4); 2.5229
(1.1); 2.5095 (18.7); 2.5050 (39.5); 2.5004 (54.9); 2.4958 (38.2); 2.4913 (17.4); 1.9877 (1.1); 1.9079 (0.9); 1.1745 (0.6); 1.0897
(1.0); 1.0679 (1.0); 0.7579 (1.0); 0.7454 (0.9); −0.0002 (2.7)
Example 1-28: ¹H-NMR (400.0 MHz, d₆-DMSO):
δ = 11.6295 (0.9); 7.6749 (2.0); 7.6085 (0.8); 7.4711 (1.7); 7.3336 (0.8); 4.0397 (16.0); 3.3095 (63.0); 2.9118 (1.4); 2.8935 (1.4);
2.8749 (0.5); 2.5229 (0.8); 2.5181 (1.2); 2.5094 (19.5); 2.5049 (42.9); 2.5003 (60.3); 2.4957 (43.1); 2.4912 (20.1); 1.1708 (1.7);
1.1525 (3.4); 1.1340 (1.7); 1.1148 (1.4); 1.1109 (1.5); 1.0992 (0.8); 1.0934 (1.4); 1.0896 (1.4); 0.6400 (1.7); 0.6288 (1.6); −0.0002
(13.9)
Example 1-29: ¹H-NMR (400.0 MHz, d₆-DMSO):
δ = 11.6711 (0.7); 8.4143 (0.8); 8.2767 (1.6); 8.1391 (0.9); 7.9189 (1.1); 7.8985 (1.7); 7.8399 (1.4); 7.8198 (0.9); 4.0561 (0.5);
4.0382 (2.1); 4.0306 (16.0); 4.0206 (1.8); 4.0028 (0.5); 3.3099 (19.0); 3.1895 (1.3); 3.1721 (1.8); 3.1543 (1.5); 3.1366 (0.5);
2.5226 (1.5); 2.5092 (20.9); 2.5049 (40.9); 2.5004 (54.3); 2.4960 (39.0); 2.4917 (19.0); 1.9878 (5.8); 1.3407 (2.8); 1.3222 (5.9);
1.3036 (2.7); 1.1923 (1.8); 1.1746 (3.4); 1.1655 (1.0); 1.1568 (2.0); 1.0269 (0.6); 1.0145 (0.8); 0.7196 (1.9); 0.7058 (2.0); 0.6916
(0.5); −0.0002 (10.8)
Example 1-30: ¹H-NMR (400.0 MHz, d₆-DMSO):
δ = 11.7409 (1.0); 7.9943 (0.8); 7.9747 (1.5); 7.9399 (2.1); 7.9199 (1.0); 7.8511 (0.9); 7.7150 (2.0); 7.5793 (1.0); 4.0265 (16.0);
3.6682 (1.0); 3.6498 (3.1); 3.6313 (3.2); 3.6128 (1.0); 3.3090 (28.7); 2.6694 (0.5); 2.6176 (0.7); 2.5046 (55.9); 2.5003 (70.6);
2.4960 (51.8); 1.9877 (1.9); 1.3642 (3.3); 1.3458 (7.0); 1.3273 (3.2); 1.1923 (0.5); 1.1745 (1.0); 1.1567 (0.5); 1.0801 (2.0);
1.0586 (1.9); 0.7547 (2.3); 0.7425 (2.2); −0.0002 (13.0)
Example 1-37: ¹H-NMR (400.0 MHz, d₆-DMSO):
δ = 11.7857 (1.1); 7.9334 (0.6); 7.9140 (0.8); 7.8969 (0.6); 7.6578 (1.1); 7.6371 (1.0); 7.5220 (0.6); 7.3856 (1.4); 7.2492 (0.6);
3.9824 (16.0); 3.3115 (48.1); 2.5230 (0.8); 2.5184 (1.1); 2.5097 (17.9); 2.5051 (39.4); 2.5005 (55.5); 2.4959 (39.4); 2.4914
(17.8); 2.4735 (7.9); −0.0002 (14.1)
Example 1-38: ¹H-NMR (400.0 MHz, d₆-DMSO):
δ = 8.1061 (0.8); 8.0870 (1.3); 8.0692 (1.0); 7.8970 (1.0); 7.7721 (1.8); 7.7607 (2.1); 7.7520 (1.7); 7.6243 (1.1); 3.9297 (16.0);
3.3095 (12.4); 3.0901 (13.2); 2.6694 (0.5); 2.5228 (1.8); 2.5181 (2.5); 2.5094 (28.2); 2.5049 (58.4); 2.5004 (80.0); 2.4958 (56.2);
2.4913 (26.2); 0.0079 (0.8); −0.0002 (22.5); −0.0085 (0.8)
Example 1-39: ¹H-NMR (400.0 MHz, d₆-DMSO):
δ = 8.2804 (0.9); 8.2607 (1.3); 8.2433 (1.0); 7.9003 (1.7); 7.8796 (1.6); 7.8690 (1.0); 7.7327 (2.1); 7.5967 (1.0); 3.9669 (16.0);
3.5139 (10.5); 3.3104 (80.6); 3.1748 (1.6); 3.1619 (1.6); 2.6740 (0.7); 2.6694 (0.9); 2.6648 (0.7); 2.5228 (3.8); 2.5181 (5.2);
2.5094 (52.7); 2.5049 (109.3); 2.5003 (150.0); 2.4957 (102.8); 2.4911 (46.4); 2.3317 (0.7); 2.3271 (0.9); 2.3224 (0.6); 1.9876
(0.6); 1.9077 (5.9); 1.2361 (0.8); 0.0081 (0.6); −0.0002 (20.8); −0.0085 (0.6)
Example 1-40: ¹H-NMR (400.0 MHz, d₆-DMSO):
δ = 7.9143 (2.3); 7.6397 (2.7); 7.6175 (2.2); 7.3770 (2.8); 3.9227 (16.0); 3.3088 (54.4); 2.9380 (4.1); 2.9203 (3.9); 2.9018 (1.9);
2.6694 (1.7); 2.5002 (303.4); 2.4959 (221.2); 2.3263 (1.7); 1.1770 (4.2); 1.1590 (8.6); 1.1410 (4.3); −0.0002 (77.2)
Example 1-41: ¹H-NMR (400.0 MHz, d₆-DMSO):
δ = 11.8887 (1.1); 8.0993 (1.5); 7.9430 (1.0); 7.8078 (2.7); 7.7950 (1.7); 7.6704 (1.4); 4.0555 (1.1); 4.0381 (3.4); 4.0203 (3.6);
4.0027 (1.1); 3.9461 (9.2); 3.4292 (1.2); 3.4110 (1.4); 3.3611 (2.9); 3.3468 (3.1); 3.3426 (5.5); 3.3096 (2040.4); 3.2729 (2.1);
3.2595 (9.3); 2.6785 (3.9); 2.6740 (8.5); 2.6693 (11.7); 2.6647 (8.4); 2.6600 (3.9); 2.6195 (1.2); 2.5612 (4.2); 2.5471 (2.8);
2.5228 (44.4); 2.5181 (62.7); 2.5094 (668.3); 2.5048 (1388.2); 2.5002 (1912.9); 2.4957 (1320.7); 2.4911 (600.8); 2.4649 (3.8);
2.4600 (5.1); 2.4553 (6.7); 2.4501 (7.0); 2.4056 (1.2); 2.3361 (4.1); 2.3316 (8.3); 2.3270 (11.7); 2.3224 (8.5); 2.0720 (2.4);
1.9876 (16.0); 1.9077 (13.6); 1.2530 (3.9); 1.2345 (8.9); 1.2165 (3.8); 1.1923 (4.6); 1.1745 (9.3); 1.1567 (4.6); 0.1458 (1.8);
0.0080 (17.5); −0.0002 (568.1); −0.0085 (16.8); −0.0505 (1.1); −0.1497 (1.7)
Example 1-42: ¹H-NMR (400.0 MHz, d₆-DMSO):
δ = 8.2076 (1.1); 7.8342 (2.7); 7.8051 (2.4); 7.7863 (1.8); 7.6979 (5.2); 7.5615 (2.4); 4.0559 (1.0); 4.0382 (2.8); 4.0203 (2.8);
4.0026 (0.9); 3.7961 (8.4); 3.5613 (2.0); 3.5428 (5.2); 3.5243 (5.3); 3.5076 (2.1); 3.4229 (0.7); 3.3778 (1.0); 3.3614 (1.5); 3.3443
(2.7); 3.3099 (734.5); 3.2789 (1.7); 3.2583 (1.0); 2.6740 (4.1); 2.6693 (5.6); 2.6646 (4.1); 2.6601 (2.1); 2.6302 (0.7); 2.5693
(1.2); 2.5227 (24.1); 2.5181 (34.2); 2.5094 (325.9); 2.5048 (662.1); 2.5003 (900.8); 2.4957 (622.0); 2.4912 (282.5); 2.4410 (1.2);
2.3316 (3.7); 2.3270 (5.3); 2.3224 (3.7); 2.1781 (0.7); 2.0719 (0.8); 1.9876 (12.3); 1.9073 (7.0); 1.2362 (13.8); 1.2240 (16.0);
1.2056 (7.5); 1.1923 (4.2); 1.1745 (7.6); 1.1567 (3.7); 1.1133 (0.7); 0.8539 (1.6); 0.8365 (0.6); 0.1459 (0.7); 0.0080 (6.8); −0.0002
(209.6); −0.0085 (6.7); −0.1497 (0.6)
Example 1-49: ¹H-NMR (400.0 MHz, d₆-DMSO): δ =
7.904 (0.66); 7.885 (0.88); 7.795 (1.36); 7.775 (0.93); 7.611 (0.58); 7.474 (1.35); 7.338 (0.69); 4.014 (16); 3.309 (38.07); 2.669 (0.64); 2.523
(1.94); 2.518 (2.95); 2.51 (36.77); 2.505 (78.98); 2.5 (109.62); 2.496 (76.69); 2.491 (34.21); 2.426 (8.5); 2.327 (0.66); 0 (15.6)
Example 1-50: ¹H-NMR (400.0 MHz, d₆-DMSO): δ =
11.958 (0.9); 8.2 (0.6); 8.063 (1.32); 8.045 (0.8); 8.01 (1.29); 7.926 (0.68); 4.01 (16); 3.305 (22.19); 3.097 (8.07); 2.669 (0.94); 2.522 (2.77); 2.518
(4.06); 2.509 (59.83); 2.505 (130.08); 2.5 (181.46); 2.495 (127.32); 2.491 (58.04); 2.327 (1.11); 0.008 (3.08); 0 (89.77); −0.008 (2.67)
Example 1-51: ¹H-NMR (400.0 MHz, d₆-DMSO): δ =
12.036 (0.97); 8.194 (0.61); 8.074 (1.13); 8.053 (0.84); 7.94 (0.6); 7.803 (1.34); 7.666 (0.7); 4.028 (16); 3.571 (8.8); 3.301 (23.52); 2.674 (0.75);
2.669 (1.08); 2.664 (0.74); 2.55 (0.55); 2.522 (3.09); 2.518 (4.34); 2.509 (58.23); 2.504 (126.06); 2.5 (175.99); 2.495 (123.31); 2.49 (55.64);
2.456 (0.51); 2.331 (0.76); 2.326 (1.05); 2.322 (0.76); 0.008 (0.69); 0 (26.33); −0.008 (0.84)
Example 1-52: ¹H-NMR (400.0 MHz, CDCl3): δ =
7.775 (3.97); 7.772 (3.32); 7.47 (0.93); 7.332 (2.04); 7.26 (29.31); 7.195 (0.99); 4.144 (16); 2.963 (0.81); 2.944 (2.5); 2.926 (2.59); 2.907 (0.99);
1.257 (3.42); 1.239 (6.98); 1.22 (3.25); 1.216 (0.8); 0 (12.32)
Example 1-53: ¹H-NMR (400.0 MHz, d₆-DMSO): δ =
11.966 (0.87); 8.098 (0.94); 8.028 (0.88); 4.038 (0.72); 4.02 (0.93); 4.012 (16); 3.314 (11.93); 3.29 (1.28); 3.271 (1.27); 2.523 (1.31); 2.518 (1.82);
2.51 (25.42); 2.505 (55.43); 2.5 (77.74); 2.496 (54.39); 2.491 (24.2); 2.455 (0.57); 2.45 (0.7); 2.446 (0.51); 1.988 (3.4); 1.332 (1.47); 1.314
(3.02); 1.295 (1.43); 1.192 (0.96); 1.174 (1.9); 1.157 (0.94); 0.008 (1.45); 0 (49.32); −0.008 (1.5)
Example 1-54: ¹H-NMR (400.0 MHz, d₆-DMSO): δ =
12.042 (0.98); 8.104 (0.76); 8.083 (0.6); 7.941 (0.58); 7.805 (1.33); 7.668 (0.67); 4.031 (16); 3.695 (1.14); 3.676 (1.17); 3.313 (4.96); 2.518 (0.64);
2.51 (8.92); 2.505 (19.32); 2.5 (26.8); 2.496 (18.67); 2.491 (8.35); 1.269 (1.53); 1.251 (3.17); 1.232 (1.53); 0 (8.17)
Example 1-61: ¹H-NMR (400.0 MHz, d₆-DMSO): δ =
11.891 (0.6); 7.845 (0.74); 7.834 (1.19); 7.623 (0.51); 7.486 (1.18); 7.349 (0.58); 4.04 (16); 3.309 (17.41); 2.523 (0.93); 2.518 (1.43); 2.509 (20.81);
2.505 (45.5); 2.5 (63.89); 2.496 (44.28); 2.491 (19.4); 2.45 (0.57); 2.422 (4.44); 0.008 (1.2); 0 (45.19); −0.003 (1.91); −0.004 (0.69); −0.005
(0.51); −0.008 (1.3)
Example 1-62: ¹H-NMR (400.0 MHz, d₆-DMSO): δ =
11.95 (0.97); 8.119 (0.85); 8.042 (0.88); 8.009 (0.55); 7.982 (0.73); 4.032 (16); 3.313 (14.24); 3.091 (5.78); 2.523 (0.75); 2.518 (1.05); 2.51 (14.9);
2.505 (32.81); 2.5 (45.95); 2.496 (32.3); 2.491 (14.95); 2.451 (1.03); 0.008 (0.52); 0 (17.37); −0.008 (0.58)
Example 1-63: ¹H-NMR (400.0 MHz, d₆-DMSO): δ =
12.024 (1.45); 8.156 (0.57); 8.136 (1.19); 8.109 (1.47); 8.088 (0.65); 7.945 (0.64); 7.809 (1.41); 7.672 (0.71); 4.052 (16); 4.033 (0.52); 3.578
(7.24); 3.315 (24.01); 2.523 (1.12); 2.518 (1.71); 2.509 (22.57); 2.505 (48.42); 2.5 (66.89); 2.496 (46.84); 2.491 (21.1); 2.45 (0.5); 1.908 (0.63);
0 (14.66)
Example 1-64: ¹H-NMR (400.0 MHz, d₆-DMSO): δ =
11.895 (0.69); 7.847 (1.98); 7.623 (0.63); 7.487 (1.38); 7.35 (0.67); 4.039 (16); 3.31 (24.78); 2.947 (1.27); 2.929 (1.29); 2.523 (1.32); 2.518 (1.97);
2.51 (25.95); 2.505 (55.92); 2.5 (77.48); 2.496 (53.77); 2.491 (23.63); 2.45 (0.51); 1.18 (1.52); 1.162 (3.05); 1.143 (1.55); 0.008 (1.65); 0.005
(0.58); 0 (57.33); −0.006 (0.78); −0.007 (0.67); −0.008 (1.71)
Example 1-65: ¹H-NMR (400.0 MHz, d₆-DMSO): δ =
11.952 (1.03); 8.144 (0.84); 8.072 (0.51); 8.051 (0.95); 8.008 (0.74); 5.754 (6.01); 4.03 (16); 3.312 (32.93); 3.29 (0.97); 3.273 (1.14); 3.255 (1.07);
3.238 (0.51); 2.523 (0.84); 2.518 (1.19); 2.51 (23.97); 2.505 (54.05); 2.5 (76.85); 2.496 (55.23); 2.491 (26.33); 2.448 (1.56); 2.327 (0.52);
1.358 (1.55); 1.34 (3.27); 1.321 (1.6); 0.008 (0.63); 0 (29.26); −0.008 (1.13)
Example 1-66: ¹H-NMR (400.0 MHz, d₆-DMSO): δ =
12.024 (2); 8.178 (0.76); 8.158 (1.87); 8.135 (2.05); 8.115 (0.84); 7.945 (0.94); 7.809 (2.09); 7.673 (1.03); 4.052 (16); 4.032 (0.78); 3.736 (0.8);
3.718 (2.2); 3.699 (2.26); 3.681 (0.89); 3.408 (8.24); 2.523 (1.56); 2.518 (2.34); 2.51 (28.84); 2.505 (61.43); 2.501 (84.52); 2.496 (58.95); 2.492
(26.44); 2.451 (0.53); 2.327 (0.52); 1.908 (2.96); 1.285 (2.36); 1.266 (4.74); 1.248 (2.44); 0 (13.92)
Example 1-73: ¹H-NMR (400.0 MHz, d₆-DMSO): δ =
11.838 (1.13); 7.83 (0.9); 7.81 (1.38); 7.747 (0.84); 7.728 (0.56); 7.616 (0.75); 7.479 (1.67); 7.342 (0.82); 4.072 (16); 3.308 (32.82); 2.669 (0.51);
2.523 (1.56); 2.518 (2.23); 2.509 (28.29); 2.505 (60.63); 2.5 (84.02); 2.496 (59.23); 2.491 (26.7); 2.389 (7.95); 0.008 (1.28); 0 (41.59); −0.008
(1.17)
Example 1-74: ¹H-NMR (400.0 MHz, d₆-DMSO):
δ = 11.8934 (2.0); 8.3097 (0.8); 8.1727 (1.5); 8.0599 (1.1); 8.0377 (1.5); 7.8836 (0.9); 7.8618 (0.7); 4.0630 (16.0); 3.3137 (92.6);
3.0611 (10.2); 2.6739 (0.8); 2.6693 (1.1); 2.6649 (0.8); 2.5093 (67.9); 2.5048 (133.1); 2.5003 (178.2); 2.4958 (128.5); 2.4914
(61.6); 2.4668 (0.9); 2.3316 (0.8); 2.3271 (1.0); 2.3226 (0.8); 1.9078 (1.0); 0.0079 (0.7); −0.0002 (15.6)
Example 1-75: ¹H-NMR (400.0 MHz, d₆-DMSO):
δ = 11.9573 (0.6); 8.0873 (0.6); 8.0668 (0.8); 8.0024 (0.8); 7.7909 (0.6); 4.6702 (16.0); 4.0858 (3.6); 3.7444 (0.6); 3.7272 (0.6);
3.5478 (3.4); 2.5073 (63.7); 1.1028 (1.1); 1.0860 (0.6); −0.0002 (7.0)
Example 1-76: ¹H-NMR (400.0 MHz, d₆-DMSO): δ =
11.845 (1.1); 7.845 (1.01); 7.824 (1.49); 7.752 (0.93); 7.732 (0.73); 7.621 (0.84); 7.484 (1.79); 7.347 (0.91); 4.072 (16); 3.309 (22.23); 2.934
(0.71); 2.916 (1.87); 2.898 (1.91); 2.879 (0.72); 2.523 (1.04); 2.51 (23.62); 2.505 (50.6); 2.5 (69.98); 2.496 (49.39); 2.492 (22.64); 1.201 (2.17);
1.182 (4.37); 1.164 (2.23); 0.008 (0.79); 0 (25.78); 0 (26.27); −0.008 (0.89)
Example 1-77: ¹H-NMR (400.6 MHz, d₆-DMSO): δ =
11.907 (1.26); 8.316 (0.51); 8.179 (1.11); 8.066 (1); 8.045 (1.52); 7.889 (0.71); 7.87 (0.59); 4.062 (16); 3.322 (71.9); 3.271 (0.71); 3.253 (0.65);
3.22 (0.94); 3.201 (1); 3.186 (0.58); 2.524 (1.42); 2.519 (1.95); 2.51 (18.46); 2.506 (38.47); 2.501 (53.07); 2.497 (37.86); 2.492 (17.47); 1.908
(0.81); 1.392 (2.57); 1.374 (5.24); 1.355 (2.46); 0 (2.75)
Example 1-78: ¹H-NMR (400.6 MHz, d₆-DMSO):
δ = 11.9547 (3.0); 8.1145 (1.7); 8.0944 (2.4); 8.0249 (2.0); 8.0044 (1.4); 7.9278 (1.3); 7.7916 (2.8); 7.6554 (1.4); 4.0838 (16.0);
4.0628 (1.2); 3.7147 (1.3); 3.6968 (3.4); 3.6786 (3.4); 3.6603 (1.3); 3.3498 (1.4); 3.3246 (83.1); 2.5054 (50.1); 2.5012 (64.4);
2.4972 (49.3); 1.3078 (3.6); 1.2898 (7.0); 1.2718 (3.6); −0.0002 (1.0)
Example 2-1: ¹H-NMR (400.0 MHz, CDCl3): δ =
7.782 (0.64); 7.762 (1.18); 7.717 (1.18); 7.697 (0.67); 7.518 (0.55); 7.504 (0.93); 7.366 (2.07); 7.26 (99.7); 7.228 (1.08); 6.996 (0.53); 4.505
(1.09); 4.487 (3.51); 4.469 (3.55); 4.451 (1.15); 2.795 (8.78); 2.306 (16); 1.655 (4); 1.637 (8.77); 1.619 (3.98); 0.008 (1.1); 0 (41.08); −0.008
(1.35)
Example 2-2: ¹H-NMR (400.0 MHz, d₆-DMSO): δ =
11.653 (1.38); 7.996 (0.99); 7.913 (0.74); 7.893 (1.22); 7.858 (0.61); 7.843 (1.5); 7.823 (0.84); 4.381 (1.22); 4.362 (3.94); 4.344 (4.01); 4.326
(1.24); 3.308 (63.88); 3.004 (16); 2.674 (0.83); 2.669 (1.17); 2.665 (0.79); 2.588 (9.37); 2.523 (4.05); 2.518 (5.99); 2.509 (67.99); 2.505 (144.81);
2.5 (198.98); 2.496 (137.97); 2.491 (61.37); 2.332 (0.8); 2.327 (1.2); 2.322 (0.85); 1.495 (4.44); 1.476 (10.14); 1.458 (4.38); 0.008 (1.03);
0 (33.01); −0.008 (1)
Example 2-3: ¹H-NMR (400.0 MHz, d₆-DMSO): δ =
11.725 (2.33); 8.062 (0.79); 8.04 (0.99); 7.94 (1.78); 7.92 (2.29); 7.784 (2.61); 7.648 (1.23); 4.396 (1.93); 4.378 (6.18); 4.36 (6.2); 4.342 (1.93);
3.953 (0.62); 3.448 (22.11); 3.307 (82.43); 2.749 (16); 2.678 (0.56); 2.674 (1.21); 2.669 (1.7); 2.665 (1.21); 2.66 (0.6); 2.554 (0.74); 2.55 (1.08);
2.545 (1.09); 2.54 (1.19); 2.523 (5.51); 2.518 (7.94); 2.509 (97.61); 2.505 (210.76); 2.5 (294.34); 2.496 (205.42); 2.491 (91.2); 2.449 (0.56);
2.444 (0.58); 2.336 (0.6); 2.332 (1.32); 2.327 (1.73); 2.322 (1.25); 2.318 (0.64); 1.499 (6.55); 1.488 (0.97); 1.481 (15.03); 1.463 (6.47); 0.008
(1.61); 0 (55.05); −0.008 (1.58)
Example 2-4: ¹H-NMR (400.0 MHz, CDCl3): δ =
7.734 (1.82); 7.713 (1.8); 7.523 (1.88); 7.518 (1.48); 7.385 (4.08); 7.259 (231.31); 7.247 (2.6); 6.995 (1.28); 5.298 (2.5); 4.501 (1.83); 4.483
(5.75); 4.464 (5.86); 4.446 (2); 2.781 (3.06); 2.771 (12.83); 2.763 (7.54); 2.744 (6.31); 2.726 (2.13); 2.042 (1.22); 1.657 (6.23); 1.639 (13.17);
1.62 (6.2); 1.549 (2.26); 1.331 (0.56); 1.284 (0.96); 1.275 (0.87); 1.258 (2.45); 1.234 (7.93); 1.215 (16); 1.197 (7.45); 0.882 (1.4); 0.864 (0.65);
0.008 (3.22); 0 (94.1); −0.008 (2.86)
Example 2-5: ¹H-NMR (400.0 MHz, d₆-DMSO): δ =
11.658 (2.62); 8.018 (0.79); 7.923 (1.2); 7.902 (2.04); 7.857 (2.63); 7.837 (1.41); 5.753 (0.64); 4.381 (2.1); 4.363 (6.57); 4.345 (6.61); 4.326
(2.09); 3.36 (1.48); 3.31 (147.53); 3.242 (1.06); 3.227 (1.35); 3.208 (1.25); 3.127 (1.46); 3.108 (1.59); 3.094 (1.02); 3.075 (0.93); 2.674 (1.24);
2.669 (1.68); 2.665 (1.25); 2.566 (12.77); 2.55 (3.76); 2.523 (9.33); 2.518 (11.63); 2.509 (91.56); 2.505 (188.98); 2.5 (258.72); 2.496 (182.09);
2.491 (83.39); 2.439 (0.56); 2.336 (0.58); 2.332 (1.13); 2.327 (1.54); 2.322 (1.05); 1.494 (7.27); 1.476 (16); 1.458 (7.09); 1.301 (5.62); 1.283
(11.89); 1.264 (5.31); 0 (11.34)
Example 2-6: ¹H-NMR (400.0 MHz, CDCl3): δ =
7.971 (1); 7.952 (3.22); 7.922 (1.79); 7.902 (0.79); 7.814 (2.84); 7.676 (1.43); 7.518 (1.29); 7.259 (209.94); 6.995 (1.08); 4.515 (1.03); 4.496
(2.82); 4.478 (2.89); 4.46 (1.07); 3.345 (1.6); 3.326 (4.87); 3.307 (5); 3.288 (1.56); 2.869 (16); 1.66 (4.56); 1.642 (9.29); 1.624 (4.63); 1.565 (0.98);
1.446 (5.4); 1.428 (11.03); 1.409 (5.05); 1.33 (1); 1.284 (1.24); 1.255 (1.35); 0.008 (2.95); 0 (83.48); −0.008 (3.21)
Example 2-13: ¹H-NMR (400.0 MHz, CDCl3): δ =
7.776 (0.71); 7.756 (1.67); 7.728 (2.13); 7.708 (0.9); 7.522 (0.87); 7.384 (1.93); 7.26 (26.96); 7.246 (0.99); 4.513 (1.12); 4.494 (3.67); 4.476
(3.7); 4.458 (1.16); 3.234 (0.66); 3.215 (2.22); 3.196 (2.25); 3.178 (0.69); 2.331 (16); 2.279 (1.29); 1.653 (3.97); 1.634 (8.64); 1.616 (3.91); 1.259
(2.77); 1.24 (6.6); 1.222 (2.76); 0 (11.06)
Example 2-14: ¹H-NMR (400.0 MHz, d₆-DMSO): δ =
11.692 (1.42); 8.122 (0.85); 7.95 (0.61); 7.93 (1.17); 7.892 (1.62); 7.872 (0.8); 4.382 (1.13); 4.364 (3.67); 4.345 (3.75); 4.327 (1.17); 3.36 (0.71);
3.31 (123.1); 3.045 (16); 3.028 (0.59); 2.884 (0.58); 2.867 (0.51); 2.674 (0.59); 2.669 (0.84); 2.665 (0.59); 2.555 (0.52); 2.551 (0.65); 2.523
(2.39); 2.518 (3.56); 2.51 (49.94); 2.505 (108.7); 2.5 (152.09); 2.496 (105.57); 2.491 (46.48); 2.45 (0.52); 2.332 (0.71); 2.327 (0.99); 2.323
(0.68); 1.496 (4.41); 1.478 (10.45); 1.46 (4.34); 1.243 (2.28); 1.224 (5.68); 1.206 (2.3); 0.008 (1.4); 0 (53); −0.008 (1.59)
Example 2-15: ¹H-NMR (400.0 MHz, d₆-DMSO): δ =
11.786 (2.88); 8.099 (0.76); 8.079 (0.95); 7.961 (1.95); 7.941 (1.51); 7.914 (1.19); 7.778 (2.66); 7.641 (1.33); 4.398 (1.87); 4.38 (6.21); 4.362
(6.32); 4.344 (1.99); 3.442 (23.78); 3.31 (71.85); 3.26 (0.53); 3.243 (0.81); 3.224 (2.56); 3.206 (2.61); 3.188 (0.82); 2.674 (0.55); 2.669 (0.78);
2.665 (0.57); 2.523 (2.17); 2.518 (3.27); 2.509 (46.2); 2.505 (100.26); 2.5 (140.15); 2.496 (97.51); 2.491 (43.24); 2.455 (0.96); 2.45 (1.19); 2.446
(0.82); 2.332 (0.66); 2.327 (0.9); 2.322 (0.66); 1.508 (6.92); 1.49 (16); 1.471 (6.85); 1.283 (3.68); 1.265 (9.36); 1.246 (3.68); 0.008 (1.44);
0 (52.54); −0.009 (1.58)
Example 2-16: ¹H-NMR (400.0 MHz, CDCl3): δ =
7.697 (13.47); 7.536 (1.83); 7.519 (0.89); 7.398 (4.27); 7.26 (151.86); 7.21 (1.21); 6.996 (0.85); 5.299 (2.93); 4.495 (1.8); 4.476 (5.74); 4.458
(5.82); 4.44 (1.87); 3.234 (1.21); 3.215 (4.19); 3.196 (4.29); 3.178 (1.36); 2.791 (2.11); 2.773 (6.77); 2.754 (6.92); 2.736 (2.25); 1.649 (6.3); 1.63
(13.48); 1.612 (6.48); 1.562 (1.18); 1.258 (7.71); 1.239 (16); 1.23 (4.76); 1.221 (7.78); 1.211 (10.55); 1.192 (4.55); 0.008 (1.64); 0 (62.7); −0.008
(2.04); −0.05 (0.51)
Example 2-17: ¹H-NMR (400.0 MHz, d₆-DMSO): δ =
11.702 (3.4); 8.105 (0.58); 7.957 (1.15); 7.938 (1.79); 7.898 (2.55); 7.878 (1.29); 4.384 (1.92); 4.366 (6.16); 4.348 (6.2); 4.329 (1.96); 3.408
(4.77); 3.287 (1.05); 3.269 (1.21); 3.254 (1.57); 3.236 (1.45); 3.115 (1.21); 3.096 (1.34); 3.081 (1.01); 3.063 (0.95); 3.003 (0.58); 2.91 (0.6); 2.892
(0.74); 2.873 (0.59); 2.67 (0.57); 2.523 (2.07); 2.518 (2.96); 2.51 (34.26); 2.505 (73.09); 2.5 (100.87); 2.496 (71.3); 2.491 (32.71); 2.455
(0.85); 2.45 (0.95); 2.446 (0.67); 2.327 (0.65); 1.498 (6.96); 1.48 (16); 1.462 (6.95); 1.334 (5.11); 1.316 (11.02); 1.297 (4.99); 1.238 (4.02); 1.22
(9.38); 1.201 (3.94); 1.097 (0.51); 0.008 (1.4); 0 (46.28); −0.008 (1.61)
Example 2-25: ¹H-NMR (400.0 MHz, d₆-DMSO):
δ = 11.4970 (5.3); 7.6651 (5.7); 7.5989 (1.8); 7.4617 (4.1); 7.3241 (2.0); 4.4055 (2.5); 4.3875 (7.8); 4.3694 (7.9); 4.3512 (2.6);
3.3109 (92.1); 2.6700 (0.7); 2.5048 (90.3); 2.5004 (124.7); 2.4960 (95.3); 2.4277 (17.1); 2.3269 (0.8); 2.2239 (1.1); 1.5140 (7.4);
1.4960 (16.0); 1.4778 (7.4); 1.1276 (1.2); 1.1124 (3.9); 1.0906 (3.8); 0.6604 (4.2); 0.6483 (4.2); −0.0002 (20.4)
Example 2-26: ¹H-NMR (400.0 MHz, d₆-DMSO):
δ = 11.5506 (0.6); 8.4204 (0.9); 8.2828 (2.0); 8.1452 (1.0); 7.9244 (1.2); 7.9043 (1.8); 7.8322 (1.3); 7.8122 (0.9); 4.3953 (1.4);
4.3771 (4.5); 4.3590 (4.6); 4.3408 (1.5); 3.3111 (15.5); 3.0501 (16.0); 2.5228 (1.4); 2.5180 (2.0); 2.5094 (19.3); 2.5049 (39.1);
2.5004 (52.9); 2.4959 (37.0); 2.4914 (17.0); 2.0912 (0.6); 1.5076 (5.0); 1.4895 (10.9); 1.4714 (4.8); 1.1359 (0.9); 1.1262 (0.7);
1.0041 (0.7); 0.9916 (0.8); 0.7379 (0.6); 0.7226 (1.7); 0.7149 (1.4); 0.7073 (1.6); 0.6910 (0.6); −0.0002 (9.7)
Example 2-27: ¹H-NMR (400.0 MHz, d₆-DMSO):
δ = 11.6185 (0.9); 7.9585 (0.6); 7.9220 (1.3); 7.9027 (0.7); 7.8503 (0.8); 7.7148 (1.8); 7.5785 (0.9); 4.3976 (1.2); 4.3794 (4.0);
4.3612 (4.0); 4.3430 (1.3); 3.5516 (16.0); 3.3127 (55.9); 2.6691 (0.6); 2.5230 (1.4); 2.5182 (2.1); 2.5095 (24.1); 2.5050 (50.8);
2.5004 (70.5); 2.4959 (49.0); 2.4913 (22.3); 1.9877 (0.8); 1.5026 (4.5); 1.4845 (10.3); 1.4663 (4.4); 1.0834 (1.6); 1.0612 (1.5);
0.7677 (1.4); 0.7550 (1.4); −0.0002 (3.4)
Example 2-28: ¹H-NMR (400.0 MHz, d₆-DMSO):
δ = 11.5133 (0.9); 7.6696 (7.3); 7.6086 (1.6); 7.4711 (3.7); 7.3337 (1.8); 4.4020 (2.1); 4.3838 (6.9); 4.3657 (7.0); 4.3475 (2.2);
4.1051 (0.8); 4.0870 (0.8); 3.3107 (89.4); 2.9283 (1.4); 2.9099 (4.2); 2.8915 (4.3); 2.8731 (1.5); 2.6696 (0.7); 2.6651 (0.5);
2.5502 (0.6); 2.5231 (1.9); 2.5184 (2.7); 2.5097 (38.7); 2.5051 (85.2); 2.5005 (119.6); 2.4959 (85.2); 2.4913 (39.3); 2.3319 (0.5);
2.3273 (0.7); 2.3227 (0.5); 2.1684 (0.6); 1.9878 (1.2); 1.5132 (7.1); 1.4950 (16.0); 1.4769 (7.1); 1.3132 (1.0); 1.2951 (2.0);
1.2770 (0.9); 1.2364 (1.2); 1.1745 (1.2); 1.1681 (5.3); 1.1497 (11.0); 1.1313 (5.2); 1.1173 (1.1); 1.1054 (2.8); 1.1016 (2.9);
1.0963 (1.6); 1.0900 (1.5); 1.0843 (2.8); 1.0803 (2.9); 1.0689 (1.0); 0.6665 (1.0); 0.6517 (3.6); 0.6406 (3.3); 0.6373 (2.9); 0.6256
(0.9); −0.0002 (8.6)
Example 2-29: ¹H-NMR (400.0 MHz, d₆-DMSO):
δ = 11.5628 (0.8); 8.4128 (1.7); 8.2753 (3.1); 8.1370 (1.8); 7.9186 (2.3); 7.8975 (3.2); 7.8340 (2.4); 7.8130 (1.5); 4.3916 (2.3);
4.3734 (6.9); 4.3552 (6.6); 4.3371 (2.1); 4.1055 (0.8); 4.0382 (1.1); 4.0207 (1.0); 3.3124 (98.0); 3.2069 (1.2); 3.1884 (2.6);
3.1736 (3.0); 3.1554 (2.7); 2.6694 (1.0); 2.5095 (67.8); 2.5051 (133.9); 2.5006 (178.2); 2.4961 (126.0); 2.4917 (59.2); 2.3274
(1.1); 2.0454 (1.2); 1.9879 (4.6); 1.9077 (1.2); 1.5063 (7.3); 1.4883 (16.0); 1.4701 (7.4); 1.3408 (5.8); 1.3223 (12.3); 1.3038
(6.0); 1.2363 (2.7); 1.1924 (1.4); 1.1746 (3.0); 1.1569 (2.7); 0.9953 (1.7); 0.7284 (3.6); 0.7134 (3.5); −0.0002 (8.2)
Example 2-30: ¹H-NMR (400.0 MHz, d₆-DMSO):
δ = 11.6232 (1.1); 7.9885 (0.7); 7.9676 (1.4); 7.9403 (2.8); 7.9201 (1.2); 7.8509 (1.3); 7.7150 (3.1); 7.5792 (1.5); 4.3930 (1.9);
4.3749 (6.1); 4.3567 (6.2); 4.3386 (1.9); 4.0382 (1.0); 4.0205 (1.0); 3.6699 (1.4); 3.6515 (4.9); 3.6330 (5.0); 3.6145 (1.5); 3.3090
(61.8); 2.6739 (0.6); 2.6693 (0.8); 2.6647 (0.6); 2.6166 (0.7); 2.5228 (3.1); 2.5181 (4.2); 2.5094 (43.8); 2.5048 (90.9); 2.5003
(124.8); 2.4957 (86.5); 2.4911 (39.4); 2.3316 (0.5); 2.3270 (0.7); 2.3224 (0.5); 1.9877 (4.5); 1.4994 (7.1); 1.4813 (16.0); 1.4631
(6.9); 1.3647 (5.2); 1.3463 (11.9); 1.3277 (5.1); 1.2477 (0.9); 1.1923 (1.3); 1.1745 (2.5); 1.1568 (1.2); 1.0902 (0.7); 1.0789 (2.4);
1.0755 (2.4); 1.0572 (2.3); 1.0538 (2.4); 1.0432 (0.8); 0.8584 (1.6); 0.8408 (0.5); 0.7623 (2.6); 0.7504 (2.6); 0.0080 (1.1); −0.0002
(34.1); −0.0085 (1.0)
Example 2-37: ¹H-NMR (400.0 MHz, d₆-DMSO):
δ = 11.6992 (0.6); 7.9256 (1.4); 7.9063 (2.0); 7.8890 (1.6); 7.6505 (2.7); 7.6303 (2.4); 7.5208 (1.3); 7.3840 (3.2); 7.2476 (1.4);
4.3607 (2.0); 4.3424 (6.3); 4.3242 (6.3); 4.3061 (2.1); 3.3121 (54.1); 2.6698 (0.6); 2.5231 (2.1); 2.5184 (3.1); 2.5098 (40.3);
2.5052 (87.1); 2.5006 (121.2); 2.4961 (85.1); 2.4915 (38.2); 2.4692 (18.3); 2.3273 (0.6); 1.4797 (7.2); 1.4615 (16.0); 1.4434
(7.1); 0.0081 (0.7); −0.0002 (24.4)
Example 2-38: ¹H-NMR (400.0 MHz, d₆-DMSO):
δ = 8.1029 (1.1); 8.0835 (1.7); 8.0657 (1.1); 7.8972 (1.3); 7.7619 (3.8); 7.7439 (1.9); 7.6243 (1.4); 4.3200 (1.4); 4.3019 (4.2);
4.2837 (4.3); 4.2656 (1.5); 4.0383 (1.5); 4.0205 (1.5); 3.3104 (14.2); 3.0864 (16.0); 2.6696 (0.6); 2.5353 (0.6); 2.5229 (2.5);
2.5183 (3.4); 2.5096 (38.0); 2.5050 (79.6); 2.5004 (109.8); 2.4959 (77.0); 2.4913 (35.5); 2.3273 (0.6); 1.9877 (7.0); 1.9077 (4.3);
1.4529 (4.7); 1.4348 (10.1); 1.4166 (4.7); 1.2363 (1.3); 1.1923 (1.9); 1.1745 (3.9); 1.1567 (1.9); 0.0080 (0.9); −0.0002 (30.6); −0.0085
(1.0)
Example 2-39: ¹H-NMR (400.0 MHz, d₆-DMSO):
δ = 8.2399 (2.3); 8.2215 (3.8); 8.2023 (2.3); 7.8493 (2.4); 7.8165 (4.9); 7.7959 (4.4); 7.7127 (4.9); 7.5765 (2.4); 4.2684 (3.2);
4.2503 (9.0); 4.2322 (9.1); 4.2142 (3.5); 3.4864 (25.8); 3.3109 (31.8); 3.1687 (2.0); 2.6693 (1.3); 2.5044 (158.8); 2.5004 (191.6);
2.4964 (145.5); 2.3270 (2.5); 1.9075 (2.4); 1.4240 (8.0); 1.4060 (16.0); 1.3879 (8.9); 1.2988 (1.5); 1.2589 (1.9); 1.2365 (5.0);
0.8541 (0.8); −0.0002 (32.4)
Example 2-40: ¹H-NMR (400.0 MHz, d₆-DMSO):
δ = 11.7204 (1.7); 7.9359 (2.4); 7.9183 (3.8); 7.9009 (2.4); 7.6571 (4.5); 7.6372 (3.7); 7.5194 (2.3); 7.3838 (4.3); 7.2484 (2.0);
4.3409 (2.6); 4.3242 (7.4); 4.3051 (7.4); 4.2869 (2.8); 3.3090 (49.0); 2.9597 (2.2); 2.9413 (6.4); 2.9230 (6.8); 2.9038 (2.8);
2.6687 (1.9); 2.5002 (293.9); 2.3271 (1.8); 1.4679 (7.5); 1.4498 (16.0); 1.4313 (8.1); 1.2334 (1.6); 1.1775 (7.4); 1.1591 (14.7);
1.1410 (8.1); −0.0002 (78.4)
Example 2-41: ¹H-NMR (400.0 MHz, d₆-DMSO):
δ = 8.1068 (2.0); 8.0885 (3.3); 8.0698 (2.0); 7.9403 (2.4); 7.8377 (0.6); 7.8038 (5.2); 7.7669 (4.1); 7.7464 (3.5); 7.7057 (0.6);
7.6674 (2.6); 4.2904 (2.6); 4.2722 (7.4); 4.2540 (7.7); 4.2360 (2.8); 4.0383 (1.4); 4.0206 (1.4); 3.5519 (0.6); 3.5341 (0.6); 3.3104
(49.0); 3.2792 (3.9); 3.2512 (2.9); 3.2323 (2.8); 3.2173 (1.8); 3.1990 (1.3); 3.1815 (0.6); 2.6740 (1.2); 2.6695 (1.5); 2.5092
(104.5); 2.5050 (194.7); 2.5005 (250.3); 2.4961 (176.6); 2.4918 (84.2); 2.3314 (1.1); 2.3272 (1.4); 2.3227 (1.1); 1.9877 (6.1);
1.4341 (7.8); 1.4160 (16.0); 1.3978 (8.2); 1.2987 (0.9); 1.2586 (2.0); 1.2455 (8.4); 1.2362 (6.9); 1.2272 (15.5); 1.2087 (7.9);
1.1924 (2.6); 1.1746 (3.6); 1.1568 (1.8); 0.8540 (0.8); 0.0078 (2.7); −0.0002 (52.5); −0.0084 (2.4)
Example 2-42: ¹H-NMR (400.0 MHz, d₆-DMSO):
δ = 8.2767 (1.8); 8.2576 (2.9); 8.2395 (1.9); 7.8731 (3.6); 7.8514 (4.7); 7.7140 (4.1); 7.5779 (2.0); 4.3036 (2.2); 4.2854 (6.8);
4.2673 (7.0); 4.2491 (2.6); 4.0383 (1.1); 4.0205 (1.1); 3.5835 (1.8); 3.5649 (5.2); 3.5465 (5.4); 3.5283 (2.1); 3.3105 (19.4);
2.6741 (0.8); 2.6695 (1.2); 2.6649 (0.9); 2.5391 (0.6); 2.5230 (4.3); 2.5183 (6.0); 2.5096 (65.2); 2.5051 (136.3); 2.5005 (187.8);
2.4959 (130.6); 2.4913 (60.0); 2.4746 (0.6); 2.4695 (0.6); 2.4647 (0.6); 2.3319 (0.8); 2.3273 (1.1); 2.3226 (0.8); 1.9877 (5.3);
1.9077 (5.4); 1.4445 (6.8); 1.4264 (14.1); 1.4083 (7.1); 1.3757 (0.8); 1.3568 (0.6); 1.2986 (0.8); 1.2523 (7.0); 1.2342 (16.0);
1.2155 (7.0); 1.1923 (1.9); 1.1791 (0.6); 1.1745 (3.3); 1.1567 (1.6); 0.8539 (0.8); 0.0079 (2.0); 0.0063 (0.9); 0.0054 (0.9);
−0.0002 (60.8); −0.0068 (0.8); −0.0085 (1.9)
Example 2-49: ¹H-NMR (400.0 MHz, CDCl3):
δ = 7.764 (6.0); 7.519 (0.6); 7.449 (1.5); 7.312 (3.2); 7.288 (0.6); 7.281 (0.7); 7.274 (1.0); 7.270 (1.1); 7.261 (99.8); 7.253 (0.8);
7.238 (1.0); 7.184 (0.8); 7.175 (1.8); 6.997 (0.5); 4.533 (1.4); 4.515 (4.3); 4.497 (4.4); 4.479 (1.5); 2.434 (16.0); 2.356 (2.5); 1.658 (5.4);
1.639 (11.0); 1.621 (5.6); 1.599 (1.1); 0.008 (1.1); 0.000 (31.9); −0.008 (0.9)
Example 2-50: ¹H-NMR (400.0 MHz, CDCl3): δ =
8.132 (0.89); 8 (1.75); 7.992 (1.3); 7.983 (1.51); 7.902 (1.89); 7.882 (1.32); 7.858 (1.04); 7.519 (1.68); 7.312 (0.71); 7.31 (0.84); 7.26 (298.81);
7.25 (2.51); 7.246 (1.94); 7.245 (1.75); 7.238 (1.35); 7.235 (1.24); 7.23 (1.29); 7.226 (1.19); 7.212 (1.82); 7.21 (2.71); 6.996 (1.68); 4.516 (0.77);
4.498 (2.34); 4.479 (2.42); 4.462 (0.94); 3.491 (3.07); 3.113 (16); 1.65 (4.71); 1.632 (9.95); 1.613 (5.13); 1.581 (1.19); 1.284 (0.54); 1.264
(0.51); 0.008 (3.84); 0.006 (1.8); 0 (131.12); −0.006 (2.66); −0.007 (2.35); −0.008 (4.88); −0.012 (1.21); −0.014 (1.02); −0.015 (1);−0.016
(0.93); −0.016 (0.92); −0.023 (0.72); −0.031 (0.65); −0.034 (0.59); −0.049 (0.89); −0.05 (1.13)
Example 2-51: ¹H-NMR (400.0 MHz, d₆-DMSO): δ =
11.958 (4.87); 8.222 (1.31); 8.204 (1.63); 8.08 (2.62); 8.06 (2.11); 7.941 (1.46); 7.805 (3.15); 7.668 (1.59); 4.415 (2.72); 4.397 (8.24); 4.378
(8.39); 4.36 (2.91); 3.578 (16); 3.322 (26.13); 2.6 (0.51); 2.51 (30.41); 2.505 (58.09); 2.501 (76.3); 2.496 (56.56); 2.492 (29.5); 2.376 (0.86); 2.328
(0.51); 1.908 (0.61); 1.503 (7.09); 1.485 (14.54); 1.467 (7.11); 0 (5.05)
Example 2-52: ¹H-NMR (400.0 MHz, d₆-DMSO): δ =
11.815 (4.01); 7.914 (0.97); 7.894 (1.25); 7.818 (2.55); 7.798 (1.69); 7.613 (1.3); 7.477 (2.89); 7.341 (1.5); 4.406 (2.74); 4.388 (8.88); 4.369
(9); 4.351 (2.81); 3.312 (50.97); 2.968 (1.01); 2.949 (2.73); 2.931 (2.85); 2.913 (1.15); 2.674 (0.97); 2.67 (1.36); 2.665 (1.01); 2.523 (3.6); 2.518
(5.5); 2.51 (74.19); 2.505 (159.44); 2.5 (220.37); 2.496 (153.51); 2.491 (68.91); 2.455 (1.13); 2.45 (1.5); 2.446 (1.22); 2.332 (1); 2.327 (1.36);
2.323 (0.97); 1.501 (7.28); 1.483 (16); 1.464 (7.26); 1.172 (3.24); 1.153 (6.44); 1.135 (3.27); 1.124 (0.96); 0.008 (1.66); 0 (55.84);−0.008
(1.75)
Example 2-53: ¹H-NMR (400.0 MHz, d₆-DMSO):
δ = 11.8750 (1.0); 8.2361 (1.6); 8.0993 (3.4); 8.0449 (1.3); 8.0259 (3.8); 8.0088 (3.9); 7.9895 (1.2); 7.9615 (1.8); 4.3757 (1.5);
4.3582 (4.2); 4.3400 (4.2); 4.3230 (1.5); 4.0382 (1.0); 4.0205 (1.2); 3.3098 (43.4); 3.2858 (6.5); 3.2671 (6.4); 3.2486 (2.4);
2.6696 (1.3); 2.5229 (4.3); 2.5183 (6.1); 2.5095 (73.4); 2.5050 (154.6); 2.5004 (215.2); 2.4958 (148.1); 2.4912 (66.6); 2.3272
(1.3); 1.9877 (4.9); 1.4810 (6.8); 1.4630 (14.4); 1.1118 (6.4); 1.3330 (7.3); 1.3143 (16.0); 1.2958 (7.0); 1.1924 (1.4); 1.1745
(2.6); 1.1567 (1.4); 0.0080 (2.8); −0.0002 (81.0); −0.0086 (2.2)
Example 2-54: ¹H-NMR (400.0 MHz, d₆-DMSO): δ =
11.942 (2); 8.228 (1.01); 8.207 (1.26); 8.097 (2.84); 8.076 (2.1); 7.94 (1.56); 7.804 (3.52); 7.668 (1.78); 4.409 (2.5); 4.391 (8.06); 4.373 (8.18);
4.355 (2.57); 4.019 (0.52); 3.713 (1.22); 3.695 (3.77); 3.676 (3.8); 3.658 (1.26); 3.359 (0.55); 3.309 (140.03); 2.679 (0.52); 2.674 (1.09); 2.669
(1.55); 2.665 (1.11); 2.66 (0.51); 2.55 (0.99); 2.546 (0.9); 2.523 (4.38); 2.518 (6.36); 2.509 (88.23); 2.505 (190); 2.5 (264.68); 2.496 (187.05);
2.491 (85.07); 2.336 (0.55); 2.332 (1.15); 2.327 (1.58); 2.322 (1.12); 2.318 (0.52); 1.908 (1.85); 1.5 (7.27); 1.482 (16); 1.464 (7.18); 1.269
(4.93); 1.251 (10.54); 1.232 (4.85); 0.008 (0.63); 0 (21.63); −0.008 (0.63)
Example 2-61: ¹H-NMR (400.0 MHz, d₆-DMSO): δ =
11.789 (0.78); 7.832 (4.59); 7.624 (1.36); 7.488 (3.17); 7.351 (1.57); 4.425 (2.21); 4.407 (7.28); 4.389 (7.44); 4.371 (2.36); 3.313 (44.01); 2.67
(0.6); 2.523 (1.36); 2.518 (1.97); 2.51 (31.73); 2.505 (70.06); 2.501 (98.7); 2.496 (68.26); 2.491 (29.74); 2.455 (0.71); 2.451 (0.9); 2.446 (0.73);
2.423 (13.86); 2.327 (0.66); 2.323 (0.54); 1.506 (7.04); 1.488 (16); 1.477 (0.88); 1.47 (7.03); 1.295 (0.67); 0.008 (1.25); 0 (53.01);−0.009
(1.52)
Example 2-62: ¹H-NMR (400.0 MHz, d₆-DMSO): δ =
11.848 (1.55); 8.258 (0.92); 8.12 (1.77); 8.058 (1.16); 8.038 (2.12); 7.993 (1.36); 7.983 (1.5); 7.974 (0.79); 4.411 (1.93); 4.393 (6.15); 4.375
(6.27); 4.357 (1.93); 3.311 (34.49); 3.092 (16); 2.674 (0.61); 2.67 (0.82); 2.665 (0.6); 2.523 (2.33); 2.518 (3.43); 2.51 (46.02); 2.505 (100.05);
2.5 (138.84); 2.496 (96.41); 2.491 (42.71); 2.455 (0.73); 2.45 (0.99); 2.446 (0.72); 2.332 (0.63); 2.327 (0.89); 2.323 (0.63); 1.498 (6.39); 1.488
(0.97); 1.48 (14.61); 1.462 (6.34); 0.008 (1.18); 0 (40.28); −0.009 (1.17)
Example 2-63: ¹H-NMR (400.0 MHz, d₆-DMSO): δ =
11.914 (5.85); 8.15 (1.2); 8.13 (2.85); 8.108 (3.49); 8.087 (1.51); 7.945 (1.55); 7.81 (3.19); 7.673 (1.6); 4.434 (3.14); 4.416 (10.37); 4.398 (10.6);
4.379 (3.5); 3.581 (16); 3.313 (71.82); 3.263 (0.56); 3.094 (0.55); 2.674 (1.08); 2.669 (1.51); 2.665 (1.14); 2.544 (0.83); 2.533 (0.85); 2.523
(4.25); 2.518 (6.48); 2.509 (87.05); 2.505 (187.69); 2.5 (260.68); 2.496 (183.67); 2.491 (83.21); 2.455 (1.32); 2.45 (1.82); 2.446 (1.48); 2.332
(1.2); 2.327 (1.64); 2.322 (1.21); 2.072 (0.73); 1.908 (1.31); 1.509 (7.03); 1.491 (14.66); 1.473 (6.98); 0.008 (1.03); 0 (32.97); −0.009
(1.01)
Example 2-64: ¹H-NMR (400.0 MHz, d₆-DMSO): δ =
11.796 (3.41); 7.845 (5.27); 7.625 (1.35); 7.488 (3.09); 7.352 (1.52); 4.425 (2.64); 4.407 (8.62); 4.388 (8.82); 4.37 (2.87); 3.313 (85.07); 3.281
(0.78); 3.262 (1.54); 2.967 (1); 2.949 (2.87); 2.931 (2.97); 2.913 (1.17); 2.674 (0.67); 2.67 (0.96); 2.665 (0.65); 2.523 (2.38); 2.518 (3.4); 2.51
(52.5); 2.505 (116.35); 2.501 (163.67); 2.496 (116.08); 2.491 (54.4); 2.451 (3.76); 2.332 (0.79); 2.327 (1.09); 2.323 (0.81); 1.506 (7.17); 1.488
(16); 1.47 (7.33); 1.182 (3.23); 1.164 (6.44); 1.145 (3.41); 0.008 (1.54); 0 (58.7); −0.008 (2.17); −0.05 (0.55)
Example 2-65: ¹H-NMR (400.0 MHz, d₆-DMSO): δ =
11.851 (3.1); 8.281 (0.9); 8.144 (2.02); 8.07 (1.29); 8.05 (2.28); 8.007 (2.18); 7.987 (0.74); 5.753 (3.48); 4.412 (2.63); 4.394 (8.03); 4.376 (8.01);
4.358 (2.49); 3.308 (51.28); 3.292 (1.78); 3.274 (2.42); 3.256 (2.06); 3.238 (0.73); 2.674 (0.84); 2.669 (1.13); 2.665 (0.81); 2.523 (3.61); 2.518
(5.24); 2.509 (62.91); 2.505 (134.1); 2.5 (185.44); 2.496 (128.96); 2.491 (57.39); 2.332 (0.81); 2.327 (1.13); 2.322 (0.77); 1.498 (7.2); 1.48
(16); 1.462 (7.08); 1.36 (3.86); 1.342 (7.87); 1.323 (3.7); 0.008 (1.31); 0 (44.58); −0.009 (1.27)
Example 2-66: ¹H-NMR (400.0 MHz, d₆-DMSO): δ =
11.907 (6.58); 8.148 (3.05); 8.132 (4.16); 8.111 (1.56); 7.946 (2.21); 7.809 (4.95); 7.673 (2.56); 5.753 (3.58); 4.431 (4.08); 4.413 (12.83); 4.395
(13.14); 4.377 (4.33); 3.736 (1.64); 3.718 (4.27); 3.7 (4.27); 3.682 (1.67); 3.308 (154.84); 2.674 (2.34); 2.669 (3.29); 2.664 (2.34); 2.605 (0.68);
2.6 (0.69); 2.596 (0.52); 2.523 (11.08); 2.518 (16.52); 2.509 (186.01); 2.505 (391.83); 2.5 (538.2); 2.496 (378.66); 2.491 (170.21); 2.405
(0.58); 2.401 (0.55); 2.336 (1.18); 2.332 (2.34); 2.327 (3.22); 2.322 (2.24); 2.072 (0.74); 1.507 (7.73); 1.489 (16); 1.471 (7.64); 1.285 (4.92);
1.268 (9.62); 1.249 (5.24); 0.008 (3.72); 0 (118.16); −0.008 (3.62)
Example 2-73: ¹H-NMR (400.0 MHz, d₆-DMSO):
δ = 11.7312 (2.7); 7.8291 (1.6); 7.8091 (2.3); 7.7295 (1.2); 7.7094 (0.9); 7.6171 (1.2); 7.4801 (2.7); 7.3432 (1.4); 4.4651 (1.8);
4.4469 (5.8); 4.4287 (5.8); 4.4106 (1.9); 4.1047 (1.2); 4.0866 (1.2); 3.3096 (124.1); 2.6740 (0.8); 2.6694 (1.2); 2.6648 (0.8);
2.5396 (1.0); 2.5229 (4.2); 2.5182 (5.7); 2.5095 (67.9); 2.5049 (143.6); 2.5003 (198.8); 2.4958 (137.4); 2.4912 (62.6); 2.4282
(0.5); 2.3895 (14.3); 2.3318 (0.9); 2.3271 (1.2); 2.3225 (0.9); 2.0724 (1.2); 1.5146 (7.0); 1.4965 (16.0); 1.4784 (7.0); 1.3127
(1.3); 1.2947 (2.7); 1.2765 (1.2); −0.0002 (1.4)
Example 2-75: ¹H-NMR (400.0 MHz, d₆-DMSO):
δ = 11.8398 (0.9); 8.0607 (1.6); 8.0404 (2.3); 7.9492 (1.5); 7.9288 (1.3); 7.7868 (2.0); 7.6505 (1.0); 4.4498 (1.0); 4.4316 (3.1);
4.4135 (3.2); 4.3954 (1.1); 3.5447 (16.0); 3.3082 (73.5); 2.6738 (0.7); 2.6693 (1.0); 2.6645 (0.7); 2.5225 (4.7); 2.5178 (6.5);
2.5091 (57.0); 2.5047 (114.6); 2.5001 (153.9); 2.4956 (107.8); 2.4911 (49.8); 2.3315 (0.6); 2.3269 (0.9); 1.9076 (0.6); 1.5032
(4.2); 1.4851 (9.3); 1.4670 (4.2); 0.0079 (1.0); −0.0002 (22.9); −0.0085 (0.8)
Example 2-76: ¹H-NMR (400.0 MHz, d₆-DMSO): δ =
11.734 (3.5); 7.845 (1.51); 7.825 (2.18); 7.736 (1.27); 7.719 (1); 7.622 (1.21); 7.485 (2.66); 7.348 (1.31); 4.465 (1.85); 4.447 (5.86); 4.429 (5.95);
4.411 (1.97); 3.31 (15.7); 2.935 (1.05); 2.917 (3.01); 2.898 (3.1); 2.88 (1.08); 2.523 (1.13); 2.518 (1.54); 2.51 (27.58); 2.505 (60.79); 2.5
(85.15); 2.496 (59.71); 2.491 (26.86); 1.516 (7.27); 1.497 (16); 1.479 (7.16); 1.202 (3.41); 1.184 (6.77); 1.165 (3.3); 0.008 (1.06); 0 (37.76); −0.009
(1)
Example 2-77: ¹H-NMR (400.6 MHz, d₆-DMSO): δ =
11.804 (3.11); 8.317 (0.87); 8.18 (1.85); 8.065 (1.77); 8.044 (2.97); 7.875 (1.2); 7.856 (1); 5.756 (2.22); 4.45 (2.21); 4.432 (6.58); 4.414 (6.53);
4.396 (2.09); 3.371 (0.61); 3.348 (0.8); 3.344 (0.92); 3.321 (147.23); 3.3 (1.36); 3.295 (0.98); 3.274 (1.21); 3.256 (1.09); 3.238 (0.85); 3.22 (1.62);
3.202 (1.7); 3.187 (0.99); 3.168 (0.77); 2.674 (0.56); 2.67 (0.77); 2.665 (0.53); 2.524 (3.93); 2.519 (5.79); 2.51 (47.64); 2.506 (96.69); 2.501
(130.94); 2.496 (91.41); 2.492 (40.65); 2.48 (1.21); 2.476 (0.93); 2.333 (0.56); 2.328 (0.78); 2.324 (0.53); 1.908 (1.8); 1.508 (7.33); 1.49
(16); 1.472 (7.12); 1.394 (4.4); 1.375 (8.88); 1.357 (4.11); 0.008 (0.79); 0 (21.3); −0.009 (0.63)
Example 2-78: ¹H-NMR (400.6 MHz, d₆-DMSO):
δ = 11.8469 (6.1); 8.1145 (2.7); 8.0943 (3.8); 8.0140 (3.3); 7.9939 (2.3); 7.9290 (2.0); 7.7926 (4.4); 7.6569 (2.2); 4.4720 (2.6);
4.4539 (7.6); 4.4358 (7.6); 4.4177 (2.6); 3.7188 (1.9); 3.7008 (5.1); 3.6822 (5.2); 3.6641 (1.9); 3.3760 (1.3); 3.3530 (2.2); 3.3273
(264.9); 3.2765 (0.5); 2.6701 (0.7); 2.5237 (3.8); 2.5105 (42.7); 2.5060 (87.1); 2.5014 (118.0); 2.4969 (83.3); 2.4924 (37.8);
2.3332 (0.5); 2.3286 (0.7); 1.5170 (7.8); 1.4989 (16.0); 1.4808 (7.7); 1.3113 (5.4); 1.2930 (10.6); 1.2746 (5.3); −0.0002 (2.2)
Example 3-1: ¹H-NMR (400.0 MHz, CDCl3): δ =
7.765 (0.6); 7.745 (1.24); 7.713 (1.31); 7.693 (0.61); 7.518 (0.61); 7.504 (1.1); 7.365 (2.37); 7.26 (110.75); 7.227 (1.16); 6.996 (0.61); 4.432
(1.91); 4.414 (2.54); 4.395 (1.99); 2.79 (8.62); 2.305 (16); 2.065 (1.18); 2.047 (2.14); 2.028 (2.13); 2.01 (1.26); 1.018 (4.29); 0.999 (8.94); 0.98
(4.15); 0.008 (1.31); 0 (45.01); −0.008 (1.28)
Example 3-2: ¹H-NMR (400.0 MHz, d₆-DMSO): δ =
11.635 (1.2); 7.994 (0.94); 7.9 (0.62); 7.88 (1.28); 7.857 (0.62); 7.847 (1.64); 7.827 (0.76); 4.323 (2.06); 4.305 (3.02); 4.287 (2.13); 3.31 (32.73);
3.006 (16); 2.669 (0.58); 2.587 (9.22); 2.523 (1.72); 2.518 (2.56); 2.51 (32.26); 2.505 (70.05); 2.5 (97.77); 2.496 (68.17); 2.491 (30.37); 2.327
(0.54); 1.918 (1.19); 1.9 (2.12); 1.882 (2.12); 1.864 (1.21); 0.908 (4.27); 0.89 (9.45); 0.871 (4.02); 0.008 (0.59); 0 (20.02); −0.009 (0.56)
Example 3-3: ¹H-NMR (400.0 MHz, d₆-DMSO): δ =
11.709 (2.02); 8.042 (0.88); 8.022 (1.14); 7.94 (1.98); 7.92 (2.35); 7.783 (2.55); 7.646 (1.19); 4.332 (3.05); 4.314 (4.5); 4.296 (3.16); 3.449 (22.02);
3.31 (102.47); 3.175 (0.65); 3.162 (0.66); 2.747 (16); 2.674 (1.3); 2.669 (1.84); 2.665 (1.3); 2.551 (0.5); 2.54 (0.62); 2.535 (0.75); 2.523
(5.28); 2.518 (8); 2.51 (105.64); 2.505 (228.67); 2.5 (317.05); 2.496 (221.05); 2.491 (98.27); 2.463 (0.66); 2.45 (0.99); 2.332 (1.29); 2.327 (1.81);
2.322 (1.34); 1.919 (1.69); 1.901 (3.04); 1.882 (3.12); 1.864 (1.77); 0.913 (6.63); 0.895 (14.54); 0.876 (6.17); 0.008 (1.76); 0 (61.61); −0.009
(1.8)
Example 4-1: ¹H-NMR (400.0 MHz, CDCl3): δ =
7.677 (6.48); 7.521 (1.05); 7.382 (2.45); 7.261 (15.43); 7.244 (1.19); 7.1 (1.13); 3.881 (13.55); 2.72 (12.81); 2.272 (16); 0 (6.76)
Example 4-2: ¹H-NMR (400.0 MHz, CDCl3): δ =
7.857 (0.66); 7.837 (1.38); 7.803 (1.37); 7.783 (0.66); 7.644 (2.96); 7.268 (0.7); 7.26 (67.37); 4.063 (1.58); 3.915 (0.9); 3.908 (14.64); 3.905
(3.49); 3.055 (1.21); 2.967 (0.66); 2.958 (16); 2.62 (6.52); 0.008 (0.82); 0 (30.16); −0.008 (0.93)
Example 4-3: ¹H-NMR (400.0 MHz, CDCl3): δ =
7.945 (0.87); 7.854 (3.09); 7.807 (1.93); 7.668 (0.94); 7.603 (0.77); 7.519 (0.67); 7.26 (125.38); 6.996 (0.66); 3.971 (0.5); 3.898 (0.6); 3.874
(8.69); 3.195 (16); 2.844 (11.22); 0.008 (1.27); 0 (48.95); −0.008 (1.37)
Example 4-4: ¹H-NMR (400.0 MHz, CDCl3): δ =
7.678 (5.46); 7.536 (1.25); 7.397 (2.9); 7.288 (1.73); 7.26 (81.53); 3.889 (16); 2.754 (1.62); 2.735 (5.25); 2.726 (15.87); 2.716 (4.96); 2.698
(1.55); 1.589 (0.52); 1.223 (5.31); 1.204 (10.84); 1.186 (4.93); 0.008 (1.22); 0 (28.79); −0.008 (0.79)
Example 4-5: ¹H-NMR (400.0 MHz, CDCl3): δ =
7.86 (4.53); 7.842 (1.29); 7.796 (1.85); 7.776 (1.05); 7.262 (24.93); 5.299 (0.8); 3.965 (16); 3.281 (0.56); 3.262 (0.52); 3.016 (0.93); 2.997 (0.98);
2.983 (0.79); 2.964 (0.77); 2.608 (3.34); 1.425 (3.08); 1.406 (6.35); 1.388 (2.93); 1.285 (0.77); 1.255 (0.72); 0 (9.06)
Example 4-6: ¹H-NMR (400.0 MHz, CDCl3): δ =
7.957 (1.24); 7.899 (0.89); 7.879 (2.38); 7.862 (1.52); 7.841 (0.91); 7.818 (2.72); 7.68 (1.54); 7.665 (1.82); 7.518 (2.1); 7.355 (0.51); 7.259 (384.1);
6.995 (1.99); 5.298 (3.43); 3.966 (1.1); 3.869 (12.06); 3.296 (1.61); 3.277 (5.09); 3.258 (5.26); 3.24 (1.71); 2.85 (16); 1.539 (2.37); 1.461
(1.05); 1.43 (5.1); 1.412 (10.1); 1.393 (4.94); 1.37 (1.31); 1.332 (7.88); 1.284 (10.97); 1.256 (4.17); 0.88 (0.71); 0.008 (5.04); 0 (144.57); −0.008
(5.71)
Example 4-25: ¹H-NMR (400.0 MHz, d₆-DMSO):
δ = 11.0342 (1.2); 7.8979 (1.1); 7.6361 (2.6); 7.5898 (0.6); 7.4521 (1.3); 7.3146 (0.6); 3.8047 (16.0); 3.4855 (0.5); 3.3085 (38.0);
2.5227 (1.8); 2.5180 (2.6); 2.5093 (28.2); 2.5047 (58.3); 2.5001 (80.0); 2.4956 (54.8); 2.4910 (24.8); 2.4229 (4.6); 2.4014 (0.8);
2.3269 (0.5); 1.2364 (0.9); 1.1167 (1.4); 1.0965 (1.4); 0.6673 (1.5); 0.6553 (1.4); 0.0080 (1.2); 0.0064 (0.5); −0.0002 (34.3); −0.0085
(1.0)
Example 4-28: ¹H-NMR (400.0 MHz, d₆-DMSO):
δ = 11.0592 (1.8); 7.9013 (1.8); 7.6478 (3.8); 7.6011 (0.8); 7.4647 (1.5); 7.3261 (0.7); 3.8054 (16.0); 3.3130 (161.1); 2.9099
(1.7); 2.8917 (1.7); 2.6696 (0.9); 2.5230 (2.8); 2.5095 (54.6); 2.5050 (116.8); 2.5005 (162.6); 2.4959 (115.8); 2.4914 (53.3);
2.3274 (0.8); 2.1905 (0.8); 1.9878 (1.6); 1.2375 (0.6); 1.1688 (2.1); 1.1506 (3.6); 1.1319 (2.2); 1.1113 (2.3); 1.0889 (2.2); 0.6566
(2.3); −0.0002 (27.6)
Example 4-29: ¹H-NMR (400.0 MHz, d₆-DMSO):
δ = 11.1164 (3.0); 8.4288 (0.6); 8.2920 (1.2); 8.1534 (0.7); 7.9208 (2.8); 7.9027 (1.4); 7.8295 (1.3); 7.8110 (0.8); 3.8214 (16.0);
3.3277 (114.0); 3.2046 (1.0); 3.1861 (1.4); 3.1662 (1.1); 2.6853 (0.6); 2.5254 (40.1); 2.5208 (83.0); 2.5163 (114.3); 2.5117
(79.0); 2.5072 (35.9); 2.3431 (0.7); 2.0703 (0.7); 2.0037 (1.6); 1.3551 (2.0); 1.3370 (3.8); 1.3182 (2.1); 1.2747 (0.6); 1.2522
(0.8); 1.2080 (0.8); 1.1903 (1.4); 1.1726 (1.3); 1.0397 (0.8); 0.7382 (1.4)
Example 4-30: ¹H-NMR (400.0 MHz, d₆-DMSO):
δ = 7.9772 (0.5); 7.9574 (1.2); 7.9376 (1.9); 7.7136 (0.8); 4.5780 (1.8); 3.8021 (16.0); 3.6474 (1.3); 3.6289 (1.4); 3.6107 (0.5);
3.3705 (11.5); 3.1686 (4.0); 2.5234 (1.9); 2.5100 (26.7); 2.5056 (54.0); 2.5011 (72.8); 2.4966 (51.3); 2.4921 (23.8); 1.3612 (1.3);
1.3428 (2.8); 1.3247 (1.5); 1.2356 (0.6); 1.0850 (1.2); 1.0644 (1.1); 0.7614 (1.2); 0.7487 (1.2); −0.0002 (13.2)
Example 4-37: ¹H-NMR (400.0 MHz, CDCl3):
δ = 8.1451 (0.6); 8.1257 (1.1); 8.1066 (0.7); 7.7963 (3.7); 7.6484 (1.3); 7.6277 (1.2); 7.3332 (1.0); 7.2602 (77.4); 7.1961 (2.1);
7.0590 (1.0); 3.8658 (16.0); 2.4727 (10.8); 1.5622 (0.7); 0.0080 (0.9); −0.0002 (27.2); −0.0085 (0.7)
Example 4-38: ¹H-NMR (400.0 MHz, d₆-DMSO):
δ = 8.0690 (1.1); 7.9259 (1.1); 7.8977 (0.9); 7.7766 (1.7); 7.7613 (2.4); 7.6255 (1.0); 3.7408 (16.0); 3.3106 (71.9); 3.1751 (1.7);
3.1619 (1.7); 3.0900 (8.1); 3.0503 (0.6); 2.6693 (0.8); 2.5092 (52.8); 2.5049 (102.7); 2.5004 (136.0); 2.4959 (96.5); 2.4916
(46.0); 2.3268 (0.8); 1.2372 (0.8); −0.0002 (12.6)
Example 4-39: ¹H-NMR (400.0 MHz, d₆-DMSO):
δ = 8.2608 (0.7); 7.9077 (1.3); 7.8869 (1.1); 7.8573 (0.7); 7.7217 (1.7); 7.5859 (0.8); 3.7525 (16.0); 3.7397 (0.8); 3.5804 (1.2);
3.5620 (1.2); 3.3099 (117.1); 2.6740 (0.8); 2.6694 (1.0); 2.6649 (0.7); 2.5228 (4.2); 2.5181 (5.9); 2.5094 (59.8); 2.5049 (123.2);
2.5003 (168.7); 2.4958 (116.6); 2.4912 (53.3); 2.3317 (0.7); 2.3271 (1.0); 2.3225 (0.7); 1.2552 (2.2); 1.2367 (5.2); 1.2184 (2.2);
0.0080 (1.0); −0.0002 (30.6); −0.0085 (1.0)
Example 4-40: ¹H-NMR (400.0 MHz, CDCl3):
δ = 8.1662 (0.5); 8.1466 (0.8); 8.1276 (0.6); 7.8130 (3.5); 7.6678 (1.1); 7.6472 (1.0); 7.3586 (0.7); 7.2602 (83.1); 7.2215 (1.5);
7.0843 (0.8); 3.9896 (1.5); 3.8706 (16.0); 3.8331 (1.8); 3.6488 (1.5); 2.9588 (0.8); 2.9403 (2.4); 2.9219 (2.5); 2.9034 (0.8);
1.2712 (2.2); 1.2532 (4.4); 1.2348 (2.1); 0.0079 (0.9); −0.0002 (29.5); −0.0085 (0.8)
Example 4-41: ¹H-NMR (400.0 MHz, d₆-DMSO):
δ = 8.0781 (0.8); 7.9416 (1.1); 7.9227 (0.6); 7.8055 (3.0); 7.7846 (1.1); 7.6695 (1.0); 4.0382 (0.7); 4.0204 (0.7); 3.7485 (1.7);
3.7376 (16.0); 3.6589 (1.0); 3.3441 (0.8); 3.3097 (128.8); 3.2694 (0.8); 3.2490 (0.6); 2.6740 (0.8); 2.6694 (1.0); 2.6648 (0.8);
2.5229 (4.3); 2.5181 (6.0); 2.5094 (59.3); 2.5049 (121.8); 2.5003 (166.1); 2.4958 (114.4); 2.4912 (52.1); 2.3317 (0.7); 2.3271
(1.0); 2.3225 (0.7); 1.9876 (3.0); 1.2482 (2.1); 1.2297 (4.1); 1.2114 (2.3); 1.1923 (1.2); 1.1745 (1.8); 1.1567 (0.9); 0.0080 (0.9); −0.0002
(29.1); −0.0085 (0.9)
Example 4-42: ¹H-NMR (400.0 MHz, d₆-DMSO):
δ = 8.2633 (0.9); 7.8929 (1.3); 7.8750 (1.0); 7.8552 (0.8); 7.7189 (2.1); 7.5823 (0.9); 7.4594 (0.5); 3.7397 (8.9); 3.6685 (1.8);
3.6387 (0.9); 3.5752 (1.8); 3.5641 (1.6); 3.5559 (1.8); 3.5101 (0.6); 3.4853 (0.8); 3.3590 (2.0); 3.3475 (1.6); 3.3093 (409.3);
2.6740 (2.2); 2.6694 (3.0); 2.6647 (2.2); 2.5497 (2.2); 2.5228 (13.1); 2.5181 (18.6); 2.5094 (180.5); 2.5049 (368.1); 2.5003
(502.5); 2.4957 (348.7); 2.4912 (159.5); 2.4706 (1.3); 2.4655 (1.4); 2.3317 (2.1); 2.3270 (2.9); 2.3224 (2.1); 1.3196 (1.5); 1.2982
(0.8); 1.2538 (3.7); 1.2357 (16.0); 1.2178 (4.1); 1.2013 (1.0); 0.8538 (1.4); 0.8364 (0.6); 0.0080 (3.4); −0.0002 (158.0); −0.0085
(7.0); −0.1501 (0.6)
Example 4-49: ¹H-NMR (400.0 MHz, d₆-DMSO): δ =
11.333 (0.88); 7.918 (0.83); 7.859 (0.7); 7.84 (0.92); 7.776 (1.1); 7.755 (0.72); 7.605 (0.64); 7.468 (1.32); 7.332 (0.65); 3.791 (16); 3.474 (2.91);
2.674 (0.65); 2.669 (0.91); 2.665 (0.62); 2.523 (2.01); 2.518 (3.06); 2.51 (48.55); 2.505 (107.49); 2.5 (151.19); 2.496 (105.96); 2.491 (47.61);
2.455 (1.15); 2.45 (1.42); 2.446 (1.18); 2.425 (5.59); 2.332 (0.73); 2.327 (1.04); 2.322 (0.75); 0.008 (1.02); 0 (43.65); −0.008 (1.4)
Example 4-50: ¹H-NMR (400.0 MHz, d₆-DMSO): δ =
8.063 (0.72); 7.985 (0.92); 7.926 (0.75); 3.78 (16); 3.092 (3.3); 2.523 (1.85); 2.518 (2.82); 2.51 (30.57); 2.505 (63.43); 2.5 (86.36); 2.496 (61.52);
2.491 (28.65); 2.327 (0.55)
Example 4-51: ¹H-NMR (400.0 MHz, d₆-DMSO): δ =
8.176 (1.48); 8.076 (1.77); 8.055 (1.58); 7.96 (2.24); 7.823 (1.95); 3.825 (16); 3.595 (10.57); 3.402 (63.21); 2.694 (1.86); 2.548 (4.42); 2.543
(6.94); 2.534 (108.49); 2.53 (231.87); 2.525 (318.71); 2.521 (225.86); 2.516 (103.46); 2.352 (1.86)
Example 4-52: ¹H-NMR (400.0 MHz, d₆-DMSO):
δ = 11.3456 (1.1); 7.9188 (1.0); 7.8657 (0.7); 7.8474 (1.0); 7.7935 (1.2); 7.7743 (0.8); 7.6052 (0.6); 7.4685 (1.2); 7.3325 (0.6);
3.7908 (16.0); 3.4179 (1.9); 3.1684 (5.6); 2.9482 (1.6); 2.9298 (1.6); 2.6695 (0.6); 2.5230 (1.3); 2.5183 (2.0); 2.5096 (33.3);
2.5050 (73.7); 2.5004 (103.6); 2.4958 (72.8); 2.4912 (33.0); 2.4549 (0.7); 2.4505 (0.9); 2.4459 (0.6); 2.3272 (0.7); 1.1703 (1.7);
1.1522 (3.2); 1.1342 (1.9); 0.0080 (0.7); −0.0002 (27.9); −0.0085 (0.9)
Example 4-53: ¹H-NMR (400.0 MHz, d₆-DMSO):
δ = 8.2356 (0.6); 8.0988 (1.2); 8.0019 (1.7); 7.9620 (0.9); 7.9317 (0.9); 5.7549 (2.5); 5.7532 (2.6); 3.7799 (16.0); 3.5748 (3.5);
3.2873 (1.5); 3.2691 (1.5); 2.5052 (49.2); 2.5013 (61.2); 2.4979 (46.0); 1.3319 (1.7); 1.3137 (3.2); 1.2961 (1.8); 0.0014 (8.0); −0.0002
(8.3)
Example 4-54: ¹H-NMR (400.0 MHz, d₆-DMSO):
δ = 8.1904 (0.7); 8.1708 (0.9); 8.0763 (1.0); 8.0562 (0.7); 7.9493 (1.4); 7.7989 (1.0); 4.7745 (1.3); 3.7990 (16.0); 3.7420 (0.7);
3.7242 (0.8); 3.6949 (1.3); 3.6772 (1.3); 3.6598 (0.6); 3.6191 (0.6); 3.3703 (14.0); 2.6701 (0.6); 2.5233 (3.0); 2.5100 (40.0);
2.5055 (79.0); 2.5010 (105.4); 2.4965 (74.2); 2.4920 (35.0); 2.3277 (0.6); 1.2679 (1.5); 1.2498 (2.8); 1.2322 (1.6); 1.1195 (0.8);
1.1018 (1.6); 1.0840 (0.8); 0.0080 (1.4); −0.0002 (36.1); −0.0085 (1.3)
Example 4-61: ¹H-NMR (400.0 MHz, d₆-DMSO): δ =
11.317 (1.92); 7.912 (1.73); 7.8 (4.35); 7.617 (0.87); 7.48 (1.79); 7.344 (0.9); 3.815 (16); 3.311 (53.37); 3.261 (0.82); 2.674 (0.73); 2.669 (0.99);
2.665 (0.76); 2.552 (0.59); 2.523 (2.3); 2.518 (3.39); 2.51 (52.75); 2.505 (116.08); 2.5 (162.65); 2.496 (114.42); 2.491 (53.15); 2.451 (3.86);
2.421 (9.65); 2.332 (0.83); 2.327 (1.16); 2.322 (0.84); 0.008 (1.51); 0 (61.65); −0.008 (2.21)
Example 4-63: ¹H-NMR (400.0 MHz, d₆-DMSO): δ =
11.473 (3.87); 8.107 (7.38); 7.969 (1.31); 7.946 (3.93); 7.832 (2.21); 7.696 (1.12); 3.855 (16); 3.603 (14.46); 3.338 (145.94); 2.697 (1.06); 2.528
(176.33); 2.355 (1.03); 2.015 (1.04); 1.202 (0.57)
Example 4-64: ¹H-NMR (400.0 MHz, d₆-DMSO): δ =
11.328 (2.17); 7.912 (2.21); 7.816 (2.84); 7.619 (0.75); 7.483 (1.49); 7.346 (0.7); 3.815 (16); 3.315 (46.07); 3.263 (0.82); 2.968 (0.84); 2.949
(2.25); 2.931 (2.34); 2.913 (1.01); 2.674 (0.67); 2.67 (0.94); 2.665 (0.68); 2.523 (2.27); 2.518 (3.58); 2.51 (54.71); 2.505 (116.11); 2.5 (158.97);
2.496 (112.64); 2.491 (52.4); 2.447 (3.71); 2.332 (0.8); 2.327 (1.05); 2.323 (0.77); 1.182 (2.46); 1.164 (4.91); 1.146 (2.73); 0.008 (1.35); 0 (52.7);
−0.008 (2.08)
Example 4-65: ¹H-NMR (400.0 MHz, d₆-DMSO): δ =
8.174 (1.02); 8.052 (1.27); 7.986 (1.22); 7.955 (1.36); 3.828 (12.61); 3.548 (16); 3.297 (1.3); 2.698 (0.93); 2.533 (111.33); 2.528 (151.51);
2.524 (117.84); 2.354 (0.88); 1.388 (1.61); 1.368 (2.99)
Example 4-66: ¹H-NMR (400.0 MHz, d₆-DMSO): δ =
8.098 (2.51); 7.949 (1.23); 7.805 (0.67); 5.754 (2.65); 4.135 (16); 3.824 (6.27); 3.716 (1.04); 3.698 (1.05); 2.507 (35.28); 2.503 (46.53); 2.499
(35.74); 1.282 (1.14); 1.263 (2.14); 1.245 (1.19); 0 (5.49)
Example 4-73: ¹H-NMR (400.0 MHz, d₆-DMSO): δ =
11.258 (2.68); 7.908 (2.46); 7.808 (1.75); 7.789 (2.35); 7.684 (2.11); 7.665 (1.62); 7.61 (1.19); 7.473 (2.51); 7.336 (1.22); 3.852 (16); 3.309 (29.72);
2.669 (0.58); 2.523 (1.41); 2.518 (2.12); 2.509 (31.57); 2.505 (68.77); 2.5 (96.02); 2.496 (68.07); 2.491 (30.91); 2.387 (14.1); 2.332 (0.52);
2.327 (0.68); 2.322 (0.51); 0.008 (1.48); 0 (50.83); −0.009 (1.48)
Example 4-74: ¹H-NMR (400.0 MHz, d₆-DMSO): δ =
8.201 (1.16); 8.066 (1.67); 7.951 (1.79); 7.849 (1.28); 7.828 (1.05); 3.865 (16); 3.086 (7.03); 2.698 (0.95); 2.529 (162.8); 2.354 (0.98)
Example 4-75: ¹H-NMR (400.0 MHz, d₆-DMSO):
δ = 8.0572 (2.0); 8.0369 (2.9); 7.9281 (4.6); 7.9137 (2.1); 7.7875 (2.1); 7.6509 (1.0); 3.8625 (16.0); 3.8380 (4.4); 3.7837 (15.1);
3.5457 (15.0); 3.0584 (0.7); 2.6744 (1.3); 2.6697 (1.8); 2.6653 (1.3); 2.5516 (1.8); 2.5449 (4.6); 2.5309 (9.4); 2.5097 (122.3);
2.5052 (234.2); 2.5007 (305.7); 2.4961 (214.7); 2.4916 (98.4); 2.4519 (1.3); 2.3321 (1.3); 2.3275 (1.8); 2.3229 (1.3); 0.0079
(1.3); −0.0002 (26.9); −0.0084 (0.9)
Example 4-76: ¹H-NMR (400.0 MHz, d₆-DMSO): δ =
11.263 (2.98); 7.908 (3.37); 7.823 (1.96); 7.802 (2.43); 7.689 (2.25); 7.67 (1.89); 7.615 (1.23); 7.478 (2.49); 7.34 (1.24); 3.851 (16); 3.308 (51.97);
2.933 (1.48); 2.915 (3.76); 2.897 (3.97); 2.878 (1.5); 2.669 (0.84); 2.509 (53.27); 2.505 (113.96); 2.5 (157.5); 2.496 (110.8); 2.491 (50.03);
2.327 (0.81); 1.2 (4.11); 1.182 (8.11); 1.164 (4.14); 0.008 (1.49); 0 (46.07); −0.008 (1.32)
Example 4-77: ¹H-NMR (400.0 MHz, d₆-DMSO): δ =
11.325 (4.98); 8.316 (0.92); 8.18 (1.86); 8.044 (2.62); 8.025 (2.17); 7.914 (3.12); 7.83 (2.03); 7.812 (1.72); 3.841 (16); 3.314 (108.52); 3.274
(2.19); 3.259 (1.75); 3.235 (1.04); 3.214 (1.46); 3.198 (1.43); 3.18 (1.13); 3.164 (0.82); 2.674 (1.48); 2.67 (1.94); 2.665 (1.38); 2.523 (7.61); 2.509
(130.64); 2.505 (254.8); 2.5 (331.08); 2.496 (233.82); 2.492 (114.74); 2.332 (1.64); 2.327 (2.06); 2.072 (0.76); 1.393 (3.77); 1.375 (7.41);
1.356 (3.97); 0.008 (2.62); 0.001 (36.17); 0 (52.19); −0.008 (2.17)
Example 4-78: ¹H-NMR (400.0 MHz, d₆-DMSO): δ =
8.086 (1.73); 8.066 (2.41); 7.954 (2.16); 7.928 (3.89); 7.788 (1.83); 7.652 (0.87); 3.861 (13.04); 3.774 (16); 3.698 (3.82); 3.679 (3.57); 3.661
(1.52); 2.67 (1.78); 2.523 (4.5); 2.518 (6.77); 2.51 (98.48); 2.505 (213.47); 2.501 (296.57); 2.496 (207.91); 2.492 (92.34); 2.328 (1.65); 2.072
(1.37); 1.306 (3.22); 1.288 (6.95); 1.27 (3.01); 0.008 (1.64); 0 (58.19); −0.008 (1.91)
Example 5-1: ¹H-NMR (400.0 MHz, CDCl3): δ =
7.783 (0.59); 7.685 (0.8); 7.665 (1.75); 7.633 (1.65); 7.613 (0.77); 7.503 (0.93); 7.365 (2.03); 7.26 (83.93); 7.227 (0.97); 7.21 (0.55); 4.145 (1.68);
2.76 (10.22); 2.522 (1.57); 2.513 (12.53); 2.495 (1.81); 2.468 (1.37); 2.297 (16); 2.262 (1.93); 1.55 (1.06); 1.269 (0.93); 1.038 (0.9); 0.008
(0.97); 0 (35.94); −0.008 (1.14)
Example 5-2: ¹H-NMR (400.0 MHz, CDCl3): δ =
7.842 (0.83); 7.822 (1.2); 7.741 (1.21); 7.721 (0.83); 7.519 (0.81); 7.26 (149.61); 7.21 (1.2); 6.996 (0.81); 2.95 (16); 2.56 (6.37); 2.502 (15.6);
1.548 (1.99); 0.008 (1.71); 0 (63.1); −0.008 (1.72)
Example 6-4: ¹H-NMR (400.0 MHz, CDCl3): δ =
7.7 (1.58); 7.68 (2.83); 7.667 (1.09); 7.623 (2.63); 7.603 (1.55); 7.524 (1.52); 7.386 (3.25); 7.266 (0.59); 7.266 (0.68); 7.265 (0.87); 7.264 (1.12);
7.26 (67.17); 7.248 (1.69); 2.777 (2.16); 2.759 (6.92); 2.751 (16); 2.74 (6.59); 2.722 (2.12); 1.581 (1.4); 1.243 (7.3); 1.225 (15.01); 1.206 (6.88);
0.008 (0.78); 0 (26.31); −0.008 (0.67)
Example 6-5: ¹H-NMR (400.0 MHz, CDCl3): δ =
9.616 (0.89); 7.825 (3.54); 7.805 (5.32); 7.738 (7.15); 7.718 (4.84); 7.519 (2.95); 7.295 (0.71); 7.287 (0.59); 7.283 (0.66); 7.281 (0.62); 7.28
(0.82); 7.279 (0.94); 7.278 (0.98); 7.277 (1.06); 7.276 (1.11); 7.276 (1.23); 7.275 (1.28); 7.274 (1.54); 7.273 (1.58); 7.272 (1.75); 7.272 (2.04);
7.271 (2.41); 7.27 (2.79); 7.269 (3.23); 7.269 (3.51); 7.268 (3.91); 7.267 (4.83); 7.266 (6.04); 7.265 (7.71); 7.264 (9.93); 7.26 (522.59); 7.256
(6); 7.255 (4.35); 7.254 (3.49); 7.253 (2.95); 7.252 (2.34); 7.252 (1.78); 7.251 (1.71); 7.25 (1.38); 7.249 (1.32); 7.248 (1.32); 7.248 (1.23); 7.247
(1.01); 7.246 (0.92); 7.245 (1); 7.244 (0.79); 7.244 (0.78); 7.243 (0.69); 7.242 (0.73); 7.241 (0.67); 7.24 (0.59); 7.24 (0.71); 7.239 (0.64); 7.236
(0.59); 7.232 (0.52); 7.228 (0.62); 7.21 (0.65); 6.996 (2.77); 5.298 (0.53); 3.265 (0.62); 3.247 (1.93); 3.229 (2.25); 3.214 (2.55); 3.195 (2.39);
3.177 (0.8); 2.982 (1.09); 2.963 (4.08); 2.948 (1.27); 2.944 (4.25); 2.93 (3.43); 2.925 (1.54); 2.91 (3.31); 2.892 (1.06); 2.506 (16); 1.576 (67.91);
1.49 (1.05); 1.421 (13.84); 1.402 (28.46); 1.383 (12.97); 1.37 (1); 1.33 (0.75); 1.285 (1.54); 1.256 (1.71); 0.146 (0.57); 0.008 (6.02); 0 (206.24);
−0.008 (5.61); −0.149 (0.6)
Example 6-6: ¹H-NMR (400.0 MHz, CDCl3): δ =
8.161 (0.52); 7.924 (1.71); 7.904 (2.51); 7.821 (1.51); 7.81 (1.18); 7.801 (1.07); 7.673 (1.84); 7.535 (0.94); 7.26 (73.64); 3.286 (1.79); 3.267
(5.88); 3.248 (5.99); 3.23 (1.85); 2.806 (16); 1.547 (3.33); 1.426 (5.36); 1.407 (11.09); 1.389 (5.05); 0.008 (1.08); 0 (28.46); −0.008 (0.85)
Example 7-1: ¹H-NMR (400.0 MHz, CDCl3): δ =
7.688 (0.76); 7.668 (1.42); 7.624 (1.56); 7.604 (0.83); 7.496 (1.04); 7.357 (2.44); 7.266 (0.56); 7.266 (0.68); 7.265 (0.85); 7.26 (52.32); 7.219
(1.26); 4.126 (0.93); 2.748 (10.89); 2.582 (0.99); 2.531 (16); 2.46 (0.81); 2.283 (15.03); 0.008 (0.75); 0 (22.28); −0.008 (0.61)
Example 7-2: ¹H-NMR (400.0 MHz, d₆-DMSO):
δ = 7.979 (1.0); 7.842 (0.5); 7.790 (5.8); 5.753 (1.6); 3.309 (25.9); 2.989 (16.0); 2.537 (9.3); 2.523 (1.4); 2.518 (1.8); 2.509 (19.5);
2.505 (41.8); 2.500 (58.2); 2.496 (41.3); 2.491 (19.5); 2.485 (13.1); 0.000 (13.3)
Example 7-3: ¹H-NMR (400.0 MHz, CDCl3): δ =
7.938 (1.26); 7.842 (1.97); 7.822 (0.54); 7.8 (2.82); 7.661 (1.44); 7.52 (1.23); 7.311 (0.5); 7.261 (208.56); 6.996 (1.21); 3.206 (16); 2.831 (14.52);
2.534 (13.34); 1.545 (2.44); 0.008 (2.67); 0 (92.86); −0.008 (2.76)
Example 7-4: ¹H-NMR (400.0 MHz, CDCl3): δ =
7.689 (1.07); 7.668 (2.22); 7.632 (1.94); 7.612 (0.94); 7.517 (0.93); 7.379 (2.09); 7.261 (28.01); 7.24 (1.03); 5.299 (1.21); 2.764 (1.27); 2.745
(4.06); 2.734 (11.3); 2.726 (4.32); 2.708 (1.29); 2.526 (16); 1.229 (4.49); 1.21 (9.27); 1.192 (4.23); 0 (10.2)
Example 7-11: ¹H-NMR (400.0 MHz, CDCl3): δ =
7.66 (1); 7.64 (2.9); 7.62 (2.1); 7.599 (0.76); 7.513 (1.11); 7.374 (2.53); 7.261 (31.84); 7.236 (1.28); 5.299 (2.55); 3.217 (0.86); 3.199 (2.88); 3.18
(2.94); 3.162 (0.9); 2.532 (16); 2.313 (15.92); 1.25 (1.49); 1.232 (2.93); 1.213 (1.43); 0 (13.53)
Example 7-12: ¹H-NMR (400.0 MHz, d₆-DMSO): δ =
12.191 (0.74); 8.105 (0.88); 7.854 (0.51); 7.833 (2.41); 7.823 (1.92); 3.311 (26.21); 3.175 (0.51); 3.162 (0.51); 3.03 (16); 2.846 (0.55); 2.523
(0.76); 2.518 (1.24); 2.51 (16.09); 2.505 (34.38); 2.5 (47.68); 2.496 (33.94); 2.491 (18.19); 1.209 (2.47); 1.191 (5.96); 1.172 (2.44); 0 (12.07)
Example 7-13: ¹H-NMR (400.0 MHz, d₆-DMSO): δ =
12.284 (1.91); 7.972 (2.8); 7.952 (5.13); 7.914 (6.07); 7.895 (4.46); 7.76 (5.53); 7.624 (2.74); 3.425 (40.65); 3.375 (0.71); 3.31 (156.79); 3.26
(1.2); 3.19 (4.01); 3.174 (4.57); 3.162 (2.89); 3.019 (0.57); 2.67 (1.54); 2.509 (103.43); 2.505 (199.64); 2.5 (263.74); 2.496 (199.47); 2.45 (2.82);
2.327 (1.79); 1.232 (7.42); 1.214 (16); 1.196 (7.18); 1.097 (0.77); 1.022 (0.74); 1.006 (0.65); 0 (45.05)
Example 7-14: ¹H-NMR (400.0 MHz, d₆-DMSO): δ =
12.098 (1.07); 7.667 (3.86); 7.663 (4.92); 7.638 (1.19); 7.5 (3.23); 7.363 (1.46); 5.754 (3.08); 3.31 (89.88); 3.26 (0.78); 3.087 (0.6); 3.07 (1.7);
3.051 (1.72); 3.033 (0.63); 2.795 (1.75); 2.776 (5.81); 2.758 (5.86); 2.739 (1.86); 2.674 (0.64); 2.669 (0.87); 2.665 (0.63); 2.523 (1.96); 2.518
(2.92); 2.51 (47.85); 2.505 (105.77); 2.5 (149.11); 2.496 (104.09); 2.491 (46.99); 2.486 (16.77); 2.467 (0.77); 2.455 (1.29); 2.451 (1.37); 2.446
(0.86); 2.332 (0.69); 2.327 (0.94); 2.322 (0.68); 2.072 (0.57); 1.174 (7.09); 1.166 (3.91); 1.155 (16); 1.147 (9.93); 1.137 (7.11); 1.128 (3.84);
0.008 (2.34); 0.006 (0.52); 0.006 (0.55); 0.005 (0.65); 0.004 (0.88); 0.003 (1.49); 0.002 (2.69); 0.002 (4.22); 0 (83.52); −0.003 (4.54); −0.003
(2.92); −0.004 (1.79); −0.005 (1.32); −0.006 (1.09); −0.007 (0.97); −0.008 (0.99); −0.008 (2.5); −0.011 (0.55); −0.05 (0.54)
Example 7-15: ¹H-NMR (400.0 MHz, d₆-DMSO): δ =
12.2 (1.5); 8.087 (0.69); 7.857 (1.06); 7.836 (4.77); 7.826 (4.02); 7.806 (0.88); 3.31 (124.99); 3.29 (0.62); 3.271 (1.38); 3.26 (1.29); 3.253 (1.64);
3.238 (2.02); 3.22 (1.95); 3.201 (0.56); 3.118 (0.5); 3.099 (1.68); 3.08 (1.8); 3.066 (1.33); 3.047 (1.22); 2.964 (0.58); 2.87 (0.72); 2.853 (0.89);
2.836 (0.66); 2.674 (0.8); 2.669 (1.14); 2.665 (0.82); 2.523 (2.79); 2.518 (4.14); 2.51 (63.33); 2.505 (140.43); 2.5 (198.05); 2.496 (137.85);
2.491 (63.3); 2.486 (24.91); 2.46 (0.94); 2.455 (1.39); 2.45 (1.88); 2.446 (1.55); 2.441 (0.97); 2.332 (0.98); 2.327 (1.27); 2.322 (0.96); 1.323
(7.23); 1.305 (16); 1.286 (6.94); 1.203 (5.31); 1.185 (13.1); 1.166 (5.3); 0.008 (1.44); 0 (59.62); −0.009 (1.78)
Example 7-16: ¹H-NMR (400.0 MHz, d₆-DMSO): δ =
12.285 (1.74); 7.987 (1.13); 7.967 (2.5); 7.939 (3.39); 7.918 (1.49); 7.891 (1.53); 7.754 (3.59); 7.618 (1.8); 3.495 (1.78); 3.476 (6.1); 3.458 (6.27);
3.44 (1.93); 3.31 (113.65); 3.26 (0.7); 3.166 (1.64); 3.148 (1.68); 2.674 (0.9); 2.669 (1.26); 2.665 (0.92); 2.523 (3.54); 2.518 (5.03); 2.51
(67.76); 2.505 (148.13); 2.5 (208.18); 2.496 (146.16); 2.491 (71.36); 2.47 (0.55); 2.455 (1.22); 2.45 (1.56); 2.446 (1.18); 2.332 (0.94); 2.327
(1.33); 2.322 (0.95); 1.265 (6.82); 1.247 (16); 1.228 (7.02); 1.22 (4.53); 1.202 (10.39); 1.184 (4.33); 0.008 (2.23); 0 (85.2); −0.004 (1.13); −0.005
(0.79); −0.006 (0.68); −0.007 (0.63); −0.008 (2.41)
Example 7-47: ¹H-NMR (400.0 MHz, d₆-DMSO):
δ = 12.3070 (2.4); 11.4223 (0.7); 7.7989 (6.2); 7.7790 (14.0); 7.7563 (16.0); 7.7361 (5.9); 7.5962 (5.3); 7.4592 (11.9); 7.3226
(5.8); 4.0859 (0.7); 4.0077 (0.6); 3.8043 (0.6); 3.3105 (152.1); 3.1746 (3.3); 3.1626 (3.0); 2.6742 (2.9); 2.6696 (3.6); 2.6648
(2.8); 2.5868 (5.2); 2.5094 (218.0); 2.5050 (397.9); 2.5004 (513.0); 2.4960 (361.2); 2.4915 (177.5); 2.4520 (5.4); 2.4305 (19.4);
2.4139 (76.2); 2.3930 (3.2); 2.3629 (1.2); 2.3479 (1.8); 2.3321 (2.7); 2.3272 (3.4); 2.2618 (1.7); 2.1052 (5.1); 1.9153 (0.6);
1.2989 (0.5); 1.2353 (2.3); −0.0002 (43.6)
Example 7-48: ¹H-NMR (400.0 MHz, d₆-DMSO):
δ = 12.3984 (0.7); 8.2036 (0.9); 8.0666 (1.5); 7.9723 (2.9); 7.9652 (3.2); 7.9448 (0.5); 7.9295 (1.0); 3.3267 (60.2); 3.0981 (16.0);
2.5385 (2.0); 2.5338 (2.8); 2.5251 (27.8); 2.5205 (57.2); 2.5159 (78.4); 2.5114 (53.6); 2.5068 (25.0); 2.4994 (6.9)
Example 7-49: ¹H-NMR (400.0 MHz, d₆-DMSO):
δ = 12.4960 (1.2); 8.1011 (1.2); 8.0495 (2.0); 8.0291 (1.2); 7.9414 (1.0); 7.8049 (2.4); 7.6679 (1.2); 4.0541 (1.1); 4.0363 (1.0);
3.5704 (16.0); 3.3765 (0.6); 3.3257 (79.3); 3.3134 (1.8); 3.3089 (0.9); 3.3031 (0.5); 3.2759 (1.2); 3.0732 (0.6); 2.6895 (0.7);
2.6850 (1.0); 2.5892 (0.6); 2.5759 (0.8); 2.5711 (0.9); 2.5665 (0.8); 2.5501 (0.7); 2.5387 (3.2); 2.5340 (4.9); 2.5253 (52.3);
2.5208 (109.5); 2.5162 (152.1); 2.5116 (105.5); 2.5071 (49.3); 2.5002 (5.5); 2.4954 (2.7); 2.4715 (1.2); 2.4670 (1.8); 2.4623
(1.6); 2.4574 (1.1); 2.4540 (0.7); 2.4451 (0.6); 2.4404 (0.5); 2.4173 (0.7); 2.3430 (0.9); 2.2580 (0.6); 2.0036 (4.6); 1.2081 (1.4);
1.1904 (2.7); 1.1726 (1.4)
Example 7-50: ¹H-NMR (400.0 MHz, d₆-DMSO):
δ = 7.7852 (0.8); 7.7749 (1.0); 7.4596 (0.7); 3.3497 (16.0); 2.9360 (1.0); 2.9176 (1.0); 2.5123 (3.5); 2.5077 (7.3); 2.5031 (10.1);
2.4986 (7.0); 2.4940 (3.4); 2.4845 (1.4); 1.9175 (0.6); 1.1652 (1.2); 1.1468 (2.5); 1.1364 (0.6); 1.1284 (1.2)
Example 7-51: ¹H-NMR (400.0 MHz, d₆-DMSO):
δ = 7.9641 (0.7); 7.9515 (0.8); 3.3105 (16.0); 3.2738 (0.7); 3.2553 (0.7); 2.5229 (0.6); 2.5094 (10.1); 2.5049 (21.0); 2.5004
(28.8); 2.4958 (20.0); 2.4913 (9.1); 2.4774 (2.2); 1.3241 (0.8); 1.3055 (1.6); 1.2868 (0.8)
Example 7-52: ¹H-NMR (400.0 MHz, d₆-DMSO):
δ = 8.1218 (0.9); 7.9739 (9.1); 7.9528 (1.2); 7.9095 (1.7); 7.8961 (1.2); 7.8764 (1.1); 7.7728 (3.7); 7.6365 (1.8); 4.0383 (1.7);
4.0206 (1.7); 4.0028 (1.0); 3.6786 (3.5); 3.6600 (7.9); 3.6416 (7.8); 3.6227 (3.2); 3.3074 (136.1); 3.2562 (1.0); 2.6692 (6.8);
2.5089 (156.6); 2.5046 (276.6); 2.5001 (358.5); 2.4956 (270.3); 2.4461 (1.1); 2.4045 (17.7); 2.3269 (2.1); 2.1780 (0.7); 2.1232
(1.0); 1.9875 (5.0); 1.9077 (0.5); 1.2461 (8.2); 1.2276 (16.0); 1.2090 (7.3); 1.1923 (1.7); 1.1746 (2.9); 1.1567 (1.6); 0.8735 (0.8);
0.8545 (0.5); −0.0002 (25.6)
Example 7-59: ¹H-NMR (400.0 MHz, d₆-DMSO): δ =
12.285 (11.15); 7.797 (7.79); 7.777 (16); 7.741 (11.02); 7.722 (6.04); 7.607 (7.23); 7.588 (1.37); 7.543 (1.25); 7.47 (15.69); 7.333 (7.54); 3.806
(2.57); 3.309 (308.67); 3.259 (2.39); 2.674 (3.82); 2.669 (5.18); 2.665 (3.73); 2.556 (1.87); 2.522 (21.21); 2.509 (336.55); 2.505 (677.17); 2.5
(910.54); 2.496 (647.97); 2.491 (314.67); 2.451 (8.82); 2.409 (92.72); 2.39 (5.64); 2.332 (4.77); 2.327 (6.16); 2.323 (4.7); 2.072 (8.25); 0.146
(1.44); 0.008 (16.38); 0 (391.24); −0.008 (14.27); −0.05 (2.19); −0.15 (1.69)
Example 7-60: ¹H-NMR (400.0 MHz, d₆-DMSO):
δ = 3.3107 (16.0); 3.0709 (3.6); 2.5228 (0.7); 2.5095 (13.7); 2.5050 (28.8); 2.5005 (39.6); 2.4959 (27.7); 2.4915 (12.6); 2.4611
(2.8)
Example 7-62: ¹H-NMR (400.0 MHz, d₆-DMSO): δ =
12.297 (3.89); 7.813 (2.72); 7.793 (4.79); 7.745 (2.81); 7.725 (1.74); 7.608 (2.26); 7.471 (5.25); 7.335 (2.53); 3.312 (116.32); 3.279 (1.3); 3.262
(1.7); 3.243 (0.79); 2.954 (2.05); 2.935 (6.37); 2.917 (6.6); 2.898 (2.49); 2.674 (1.25); 2.67 (1.78); 2.665 (1.3); 2.551 (0.94); 2.523 (4.29); 2.518
(6.37); 2.51 (95.36); 2.505 (211.57); 2.5 (298.93); 2.496 (212.49); 2.491 (102.95); 2.451 (6.21); 2.332 (1.45); 2.327 (1.99); 2.323 (1.49);
2.073 (0.82); 1.174 (7.7); 1.156 (16); 1.137 (7.73); 0.008 (2.67); 0.006 (0.55); 0.006 (0.59); 0.005 (0.7); 0 (94.47); −0.005 (2.23); −0.006
(1.94); −0.007 (1.75); −0.008 (3.38); −0.051 (0.56)
Example 7-71: ¹H-NMR (400.6 MHz, d₆-DMSO): δ =
7.808 (1.5); 7.789 (1.75); 7.615 (1.07); 7.595 (0.94); 7.583 (1.11); 7.446 (2.14); 7.309 (1.05); 4.152 (16); 3.187 (1.47); 3.183 (2.73); 3.179 (3.8);
3.175 (2.65); 3.171 (1.34); 2.58 (1.33); 2.551 (6.96); 2.547 (13.3); 2.542 (17.67); 2.538 (12.57); 2.533 (6.12); 2.502 (3.34); 2.393 (11.85); 2.375
(0.65); 2.047 (0.78); 0 (1.87)
Example 7-72: ¹H-NMR (400.0 MHz, d₆-DMSO):
δ = 8.3017 (0.6); 8.1636 (1.3); 8.0261 (0.7); 7.9259 (1.1); 7.9062 (1.2); 7.6232 (1.2); 7.6031 (1.1); 3.3090 (37.8); 3.0268 (16.0);
2.6739 (0.7); 2.6693 (1.0); 2.6646 (0.7); 2.5556 (0.5); 2.5507 (0.6); 2.5227 (3.6); 2.5180 (5.1); 2.5093 (54.6); 2.5048 (112.2);
2.5002 (152.6); 2.4956 (105.2); 2.4911 (47.9); 2.4561 (0.6); 2.4518 (0.6); 2.3878 (10.1); 2.3316 (0.7); 2.3270 (0.9); 2.3224 (0.6);
1.9075 (1.3); 0.0080 (1.2); −0.0002 (32.9); −0.0085 (1.0)
Example 7-74: ¹H-NMR (400.0 MHz, d₆-DMSO): δ =
12.225 (3.02); 7.802 (3.59); 7.782 (4.31); 7.625 (2.36); 7.605 (4); 7.468 (4.83); 7.331 (2.29); 3.308 (31.48); 2.922 (2.06); 2.904 (6.48); 2.885
(6.79); 2.867 (2.46); 2.67 (0.72); 2.523 (2.24); 2.51 (44.57); 2.505 (95.06); 2.5 (132.34); 2.496 (96.1); 2.491 (47.54); 2.327 (0.76); 1.193 (7.72);
1.174 (16); 1.156 (7.79); 0.008 (1.47); 0 (50.69); −0.009 (1.57)
Example 7-75: ¹H-NMR (400.0 MHz, d₆-DMSO):
δ = 8.3100 (0.6); 8.1716 (1.3); 8.0333 (0.7); 7.8209 (1.7); 7.8009 (1.9); 7.4686 (2.2); 7.4487 (2.0); 4.0881 (0.7); 4.0747 (0.8);
3.3109 (62.3); 3.2707 (1.2); 3.2518 (0.8); 3.2372 (1.2); 3.2183 (1.1); 3.1743 (3.5); 3.1613 (3.8); 3.1444 (1.0); 3.1262 (1.1);
3.1106 (0.7); 3.0927 (0.6); 2.6693 (0.6); 2.5498 (0.7); 2.5450 (0.7); 2.5046 (66.7); 2.5001 (94.3); 2.4957 (73.7); 2.3270 (0.6);
2.2695 (16.0); 1.3734 (3.2); 1.3549 (6.7); 1.3363 (3.2); 0.0080 (0.9); −0.0002 (27.5); −0.0084 (1.6)
Example 9-25: ¹H-NMR (400.0 MHz, CDCl3): δ =
7.963 (1.47); 7.942 (1.75); 7.712 (1.65); 7.692 (1.43); 7.518 (0.58); 7.449 (0.9); 7.312 (1.97); 7.271 (0.56); 7.271 (0.56); 7.268 (0.79); 7.259 (100.61);
7.174 (0.95); 6.995 (0.57); 2.423 (16); 0.008 (1.4); 0 (43.08); −0.008 (1.14)

### B. Formulation Examples

a) A dusting product is obtained by mixing 10 parts by weight of a compound of the formula (I) and/or salts thereof and 90 parts by weight of talc as an inert substance and comminuting the mixture in a hammer mill
b) A readily water-dispersible, wettable powder is obtained by mixing 25 parts by weight of a compound of the formula (I) and/or salts thereof, 64 parts by weight of kaolin-containing quartz as an inert substance, 10 parts by weight of potassium lignosulfonate and 1 part by weight of sodium oleoylmethyltaurate as a wetting agent and dispersant, and grinding the mixture in a pinned-disk mill
c) A readily water-dispersible dispersion concentrate is obtained by mixing 20 parts by weight of a compound of the formula (I) and/or salts thereof with 6 parts by weight of alkylphenol polyglycol ether (®Triton X 207), 3 parts by weight of isotridecanol polyglycol ether (8 EO) and 71 parts by weight of paraffinic mineral oil (boiling range for example about 255 to above 277 C), and grinding the mixture in a friction ball mill to a fineness of below 5 microns.
d) An emulsifiable concentrate is obtained from 15 parts by weight of a compound of the formula (I) and/or salts thereof, 75 parts by weight of cyclohexanone as a solvent and 10 parts by weight of ethoxylated nonylphenol as an emulsifier.
e) Water-dispersible granules are obtained by mixing
   75 parts by weight of a compound of the formula (I) and/or salts thereof,
   10 parts by weight of calcium lignosulfonate,
   5 parts by weight of sodium lauryl sulfate,
   3 parts by weight of polyvinyl alcohol and
   7 parts by weight of kaolin,
   grinding the mixture in a pinned-disk mill, and granulating the powder in a fluidized bed by spray application of water as a granulating liquid.
f) Water-dispersible granules are also obtained by homogenizing and precomminuting, in a colloid mill,
   25 parts by weight of a compound of the formula (I) and/or salts thereof,
   5 parts by weight of sodium 2,2′-dinaphthylmethane-6,6′-disulfonate
   2 parts by weight of sodium oleoylmethyltaurate,
   1 part by weight of polyvinyl alcohol,
   17 parts by weight of calcium carbonate and
   50 parts by weight of water,
   then grinding the mixture in a bead mill and atomizing and drying the resulting suspension in a spray tower by means of a one-phase nozzle.

### C. Biological Examples

The abbreviations used here mean:

| | | | |
|---|---|---|---|
| ABUTH | Abutilon theophrasti | ALOMY | Alopecurus myosuroides |
| AMARE | Amaranthus | AVEFA | Avena fatua |
| | retroflexus | | |
| CYPES | Cyperus serotinus | MATIN | Matricaria inodora |
| PHBPU | Pharbitis purpureum | POLCO | Polygonum convolvulus |
| SETVI | Setaria viridis | STEME | Stellaria media |
| VERPE | Veronica persica | VIOTR | Viola tricolor |

1. Pre-Emergence Herbicidal Action Against Harmful Plants Seeds of monocotyledonous and dicotyledonous weed plants and crop plants are laid out in sandy loam soil in wood-fiber pots and covered with soil. The compounds of the invention, formulated in the form of wettable powders (WP) or as emulsion concentrates (EC), are then applied to the surface of the covering soil in the form of an aqueous suspension or emulsion at a water application rate equating to 600 to 8001/ha, with addition of 0.2% wetting agent. After the treatment, the pots are placed in a greenhouse and kept under good growth conditions for the trial plants. The damage to the test plants is scored visually after a test period of 3 weeks by comparison with untreated controls (herbicidal activity in percent (%): 100% activity=the plants have died, 0% activity=like control plants). Here, numerous compounds according to the invention showed, at an application rate of 320 g or less per hectare, an activity of at least 80% against a large number of important harmful plants.

In addition, some substances are also harmless to dicotyledonous crops such as soya, cotton, oilseed rape, sugar beet or potatoes. Some of the compounds according to the invention exhibit high selectivity and are therefore suitable for controlling unwanted vegetation in agricultural crops by the pre-emergence method. The data of Tables A and B below illustrate, in an exemplary manner, the pre-emergence herbicidal action of the compounds according to the invention, the herbicidal activity being stated in percent.

TABLE A
Pre-emergence

| Example | Dosage | | | | | |
|---|---|---|---|---|---|---|
| No. | [g/ha] | ALOMY | AVEFA | CYPES | SETVI | ABUTH |
| 1-1 | 320 | 100 | 100 | 80 | 100 | 100 |
| 1-2 | 320 | 100 | 100 | 90 | 100 | 100 |
| 1-3 | 320 | 100 | 100 | 90 | 100 | 100 |
| 1-4 | 320 | 100 | 100 | 80 | 100 | 100 |
| 1-5 | 320 | 100 | 100 | 100 | 100 | 100 |
| 1-6 | 320 | 100 | 100 | 100 | 100 | 100 |
| 1-13 | 320 | 100 | 100 | 100 | 100 | 100 |
| 1-14 | 320 | 100 | 100 | 100 | 100 | 100 |
| 1-15 | 320 | 100 | 100 | 100 | 100 | 100 |
| 1-16 | 320 | 100 | 100 | 100 | 100 | 100 |
| 1-17 | 320 | 100 | 100 | 100 | 100 | 100 |
| 1-18 | 320 | 100 | 100 | 100 | 100 | 100 |
| 1-25 | 320 | 100 | 100 | 100 | 100 | 100 |
| 1-26 | 320 | 100 | 100 | 100 | 100 | 100 |
| 1-27 | 320 | 100 | 100 | 100 | 100 | 100 |
| 1-28 | 320 | 100 | 100 | 100 | 100 | 100 |
| 1-29 | 320 | 100 | 100 | 100 | 100 | 100 |
| 1-30 | 320 | 100 | 100 | 90 | 100 | 100 |
| 1-37 | 320 | 100 | 100 | 100 | 100 | 100 |
| 1-38 | 320 | 100 | 100 | 90 | 100 | 100 |
| 1-39 | 320 | 90 | 100 | 90 | 100 | 100 |
| 1-40 | 320 | 100 | 90 | 100 | 100 | 100 |
| 1-41 | 320 | 100 | 100 | 100 | 100 | 100 |
| 1-42 | 320 | 100 | 100 | 100 | 100 | 100 |
| 1-49 | 320 | 100 | 100 | 90 | 100 | 100 |
| 1-50 | 320 | 100 | 100 | 100 | 100 | 100 |
| 1-51 | 320 | 100 | 100 | 100 | 100 | 100 |
| 1-52 | 320 | 100 | 100 | 90 | 100 | 100 |
| 1-53 | 320 | 100 | 100 | 100 | 100 | 100 |
| 1-54 | 320 | 100 | 100 | 100 | 100 | 100 |
| 1-61 | 320 | 100 | 100 | 100 | 100 | 100 |
| 1-62 | 320 | 100 | 100 | 100 | 100 | 100 |
| 1-63 | 320 | 100 | 100 | 100 | 100 | 100 |
| 1-64 | 320 | 100 | 100 | 100 | 100 | 100 |
| 1-65 | 320 | 100 | 100 | 90 | 100 | 100 |
| 1-74 | 320 | 100 | 100 | 100 | 100 | 100 |
| 1-75 | 320 | | | | 100 | 90 |
| 1-78 | 320 | 100 | 100 | 100 | 100 | 100 |
| 2-1 | 320 | 90 | 100 | | 100 | 100 |
| 2-2 | 320 | 100 | 100 | 90 | 100 | 100 |
| 2-3 | 320 | 90 | 100 | 100 | 100 | 100 |
| 2-4 | 320 | 100 | 100 | 100 | 100 | 100 |
| 2-5 | 320 | 100 | 90 | 80 | 100 | 100 |
| 2-6 | 320 | 100 | 100 | 100 | 100 | 100 |
| 2-13 | 320 | 100 | 100 | 100 | 100 | 100 |
| 2-14 | 320 | 100 | 100 | 100 | 100 | 100 |
| 2-15 | 320 | 100 | 100 | 100 | 100 | 100 |
| 2-16 | 320 | 100 | 100 | 100 | 100 | 100 |
| 2-17 | 320 | 100 | 100 | 100 | 100 | 100 |
| 2-18 | 320 | 100 | 100 | 90 | 100 | 100 |
| 2-25 | 320 | 100 | 100 | 100 | 100 | 100 |
| 2-26 | 320 | 100 | 100 | 90 | 100 | 100 |
| 2-27 | 320 | 100 | 100 | 100 | 100 | 100 |
| 2-28 | 320 | 100 | 100 | 100 | 100 | 100 |
| 2-29 | 320 | 100 | 100 | 90 | 100 | 100 |
| 2-30 | 320 | 100 | 100 | 90 | 100 | 100 |
| 2-37 | 320 | 90 | 100 | 80 | 100 | 100 |
| 2-38 | 320 | 100 | 100 | 100 | 100 | 100 |
| 2-39 | 320 | | 100 | 100 | 100 | 100 |
| 2-40 | 320 | | | | 100 | 100 |
| 2-41 | 320 | 100 | 100 | 90 | 100 | 100 |
| 2-42 | 320 | 90 | 90 | 90 | 100 | 100 |
| 2-49 | 320 | 100 | 100 | 90 | 100 | 100 |
| 2-50 | 320 | 100 | 100 | 100 | 100 | 100 |
| 2-51 | 320 | 90 | 100 | 90 | 100 | 100 |
| 2-62 | 320 | 100 | 100 | 100 | 100 | 100 |
| 2-63 | 320 | 100 | 100 | 90 | 100 | 100 |
| 2-64 | 320 | 100 | 100 | 100 | 100 | 100 |
| 2-61 | 320 | 100 | 100 | 100 | 100 | 100 |
| 2-78 | 320 | 100 | 100 | 100 | 100 | 100 |
| 3-1 | 320 | 100 | 100 | | 100 | 100 |
| 3-2 | 320 | 90 | 90 | 80 | 100 | 100 |
| 3-3 | 320 | 90 | 100 | 90 | 100 | 100 |
| 4-1 | 320 | | 80 | | 100 | 100 |
| 4-2 | 320 | | 80 | 80 | 100 | 100 |
| 4-3 | 320 | 90 | 80 | 100 | 100 | 100 |
| 4-6 | 320 | | | 100 | 90 | 100 |
| 4-25 | 320 | | | | | 90 |
| 4-28 | 320 | | | | | 100 |
| 4-29 | 320 | | | | | 100 |
| 4-30 | 320 | | | 80 | | 100 |
| 4-37 | 320 | | | | | 100 |
| 4-38 | 320 | | | | | 90 |
| 4-39 | 320 | | | | 100 | 100 |
| 4-40 | 320 | | | 100 | 80 | 100 |
| 4-41 | 320 | | | | 80 | 100 |
| 4-52 | 320 | 100 | 100 | 80 | 100 | 100 |
| 4-53 | 320 | 100 | 100 | 100 | 100 | 100 |
| 4-54 | 320 | 90 | 80 | 80 | 90 | 100 |
| 4-61 | 320 | 100 | 90 | 90 | 100 | 100 |
| 4-64 | 320 | 90 | 100 | | 100 | 100 |
| 4-75 | 320 | | | 80 | 90 | 100 |
| 5-1 | 320 | | | 80 | 100 | 100 |
| 6-4 | 320 | | 90 | | 100 | 90 |
| 6-5 | 320 | 90 | 90 | | 100 | 100 |
| 6-6 | 320 | 90 | 90 | 80 | 100 | 100 |
| 7-2 | 320 | 90 | 90 | 90 | 100 | 100 |
| 7-3 | 320 | 100 | 100 | 100 | 100 | 100 |
| 7-4 | 320 | 90 | | | 100 | 100 |
| 7-11 | 320 | 80 | 90 | 90 | 100 | 100 |
| 7-12 | 320 | 100 | 100 | 100 | 100 | 100 |
| 7-13 | 320 | 100 | 100 | 100 | 100 | 100 |
| 7-14 | 320 | 100 | 100 | 100 | 100 | 100 |
| 7-15 | 320 | 100 | 100 | 100 | 100 | 100 |
| 7-16 | 320 | 100 | 100 | 100 | 100 | 100 |
| 7-47 | 320 | 90 | 90 | 80 | 100 | 100 |
| 7-50 | 320 | 100 | 100 | 90 | 100 | 100 |
| 7-64 | 320 | 100 | 100 | 100 | 100 | 100 |
| 7-62 | 320 | 100 | 90 | 80 | 100 | 100 |
| 7-75 | 320 | 100 | 100 | 100 | 100 | 100 |

TABLE B
Pre-emergence

| Example | Dosage | | | | |
|---|---|---|---|---|---|
| No. | [g/ha] | AMARE | MATIN | STEME | VIOTR |
| 1-1 | 320 | 100 | 100 | 100 | 100 |
| 1-2 | 320 | 100 | 100 | 90 | 100 |
| 1-3 | 320 | 100 | 100 | 100 | 100 |
| 1-4 | 320 | 100 | 100 | 100 | 100 |
| 1-5 | 320 | 100 | 100 | 90 | 100 |
| 1-6 | 320 | 100 | 100 | 100 | 100 |
| 1-13 | 320 | 100 | 100 | 100 | 100 |
| 1-14 | 320 | 100 | 100 | 100 | 100 |
| 1-15 | 320 | 100 | 100 | 100 | 100 |
| 1-16 | 320 | 100 | 100 | 100 | 100 |
| 1-17 | 320 | 100 | 100 | 100 | 100 |
| 1-18 | 320 | 100 | 100 | 100 | 100 |
| 1-25 | 320 | 100 | 100 | 100 | 100 |
| 1-26 | 320 | 100 | 100 | 100 | 100 |
| 1-27 | 320 | 100 | 100 | 100 | 100 |
| 1-28 | 320 | 100 | 100 | 100 | 100 |
| 1-29 | 320 | 100 | 100 | 100 | 100 |
| 1-30 | 320 | 100 | 100 | 100 | 100 |
| 1-37 | 320 | 100 | 100 | 100 | 100 |
| 1-38 | 320 | 100 | 100 | 100 | 100 |
| 1-39 | 320 | 100 | 100 | 100 | 100 |
| 1-40 | 320 | 100 | 100 | 100 | 100 |
| 1-41 | 320 | 100 | 100 | 100 | 100 |
| 1-42 | 320 | 100 | 100 | 100 | 100 |
| 1-49 | 320 | 100 | 100 | 90 | 100 |
| 1-50 | 320 | 100 | 100 | 90 | 100 |
| 1-51 | 320 | 100 | 100 | 100 | 100 |
| 1-52 | 320 | 100 | 100 | 100 | 100 |
| 1-53 | 320 | 100 | 100 | 100 | 100 |
| 1-54 | 320 | 100 | 100 | 100 | 100 |
| 1-61 | 320 | 100 | 100 | 100 | 100 |
| 1-62 | 320 | 100 | 100 | 100 | 100 |
| 1-63 | 320 | 100 | 100 | 100 | 100 |
| 1-64 | 320 | 100 | 100 | 100 | 100 |
| 1-65 | 320 | 100 | 100 | 100 | 100 |
| 1-74 | 320 | 100 | 100 | 100 | 100 |
| 1-75 | 320 | 90 | 90 | 100 | 90 |
| 1-78 | 320 | 100 | 100 | | 100 |
| 2-2 | 320 | 100 | 100 | | 100 |
| 2-1 | 320 | 100 | 100 | | 100 |
| 2-3 | 320 | 100 | 100 | 90 | 100 |
| 2-4 | 320 | 100 | 100 | 90 | 100 |
| 2-5 | 320 | 100 | 100 | 90 | 100 |
| 2-6 | 320 | 100 | 100 | 100 | 100 |
| 2-13 | 320 | 100 | 100 | 100 | 100 |
| 2-14 | 320 | 100 | 100 | 100 | 100 |
| 2-15 | 320 | 100 | 100 | 100 | 100 |
| 2-16 | 320 | 100 | 100 | 100 | 100 |
| 2-17 | 320 | 100 | 100 | 100 | 100 |
| 2-18 | 320 | 100 | 100 | 100 | 100 |
| 2-25 | 320 | 100 | 100 | 100 | 100 |
| 2-26 | 320 | 100 | 100 | 100 | 100 |
| 2-27 | 320 | 100 | 100 | 100 | 100 |
| 2-28 | 320 | 100 | 100 | 100 | 100 |
| 2-29 | 320 | 100 | 100 | 100 | 100 |
| 2-30 | 320 | 100 | 100 | 100 | 100 |
| 2-37 | 320 | 100 | 100 | 100 | 100 |
| 2-38 | 320 | 100 | 100 | 100 | 100 |
| 2-39 | 320 | 100 | 100 | 100 | 100 |
| 2-40 | 320 | 100 | 100 | 100 | 100 |
| 2-41 | 320 | 100 | 100 | 100 | 100 |
| 2-42 | 320 | 100 | 100 | 90 | 100 |
| 2-49 | 320 | 100 | 100 | 90 | 100 |
| 2-50 | 320 | 100 | 100 | 100 | 100 |
| 2-51 | 320 | 100 | 100 | 90 | 100 |
| 2-62 | 320 | 100 | 100 | 100 | 100 |
| 2-63 | 320 | 100 | 100 | 100 | 100 |
| 2-64 | 320 | 100 | 100 | 100 | 100 |
| 2-61 | 320 | 100 | 100 | 100 | 100 |
| 2-78 | 320 | 100 | 100 | 100 | 100 |
| 3-1 | 320 | 100 | 100 | 100 | 100 |
| 3-2 | 320 | 100 | 100 | 90 | 100 |
| 3-3 | 320 | 100 | 100 | | 100 |
| 4-1 | 320 | 100 | 90 | 90 | 100 |
| 4-2 | 320 | 100 | 100 | 100 | 90 |
| 4-3 | 320 | 100 | 100 | 100 | 100 |
| 4-4 | 320 | 100 | 100 | 100 | 100 |
| 4-5 | 320 | 100 | 90 | | 100 |
| 4-6 | 320 | 100 | 100 | 90 | 100 |
| 4-25 | 320 | 90 | 90 | 90 | 100 |
| 4-28 | 320 | 90 | 90 | 90 | 100 |
| 4-29 | 320 | 90 | 90 | 90 | 100 |
| 4-30 | 320 | 90 | 100 | 90 | 90 |
| 4-37 | 320 | 100 | 100 | 100 | 90 |
| 4-39 | 320 | 100 | 90 | 100 | 100 |
| 4-40 | 320 | 100 | 100 | 100 | 100 |
| 4-41 | 320 | 100 | | 80 | 90 |
| 4-52 | 320 | 100 | 100 | 100 | 100 |
| 4-53 | 320 | 100 | 100 | 100 | 100 |
| 4-54 | 320 | 100 | 100 | 100 | 100 |
| 4-61 | 320 | 100 | 100 | 90 | 100 |
| 4-64 | 320 | 100 | 100 | 100 | 100 |
| 4-75 | 320 | 100 | 100 | 100 | 90 |
| 5-1 | 320 | 100 | 100 | 90 | 100 |
| 6-4 | 320 | 100 | 90 | 90 | 100 |
| 6-5 | 320 | 100 | 90 | 90 | 100 |
| 6-6 | 320 | 90 | 100 | 90 | 100 |
| 7-1 | 320 | 100 | 80 | 80 | 100 |
| 7-2 | 320 | 100 | 90 | 80 | 100 |
| 7-3 | 320 | 100 | 100 | 100 | 100 |
| 7-4 | 320 | 100 | 80 | 90 | 100 |
| 7-11 | 320 | 100 | | 80 | 100 |
| 7-12 | 320 | 100 | 100 | 100 | 100 |
| 7-13 | 320 | 100 | 100 | 100 | 100 |
| 7-14 | 320 | 100 | 90 | 100 | 100 |
| 7-15 | 320 | 100 | 100 | 100 | 100 |
| 7-16 | 320 | 100 | 100 | 100 | 100 |
| 7-47 | 320 | 100 | 100 | 90 | 100 |
| 7-50 | 320 | 90 | 90 | 100 | 100 |
| 7-62 | 320 | 100 | 90 | 100 | 100 |
| 7-64 | 320 | | 100 | 100 | 100 |
| 7-75 | 320 | 100 | 100 | 100 | 100 |

2. Post-Emergence Herbicidal Action Against Harmful Plants

Seeds of monocotyledonous and dicotyledonous weed and crop plants are laid out in sandy loam soil in wood-fiber pots, covered with soil and cultivated in a greenhouse under good growth conditions. 2 to 3 weeks after sowing, the test plants are treated at the one-leaf stage. The compounds of the invention, formulated in the form of wettable powders (WP) or as emulsion concentrates (EC), are then sprayed onto the green parts of the plants in the form of an aqueous suspension or emulsion at a water application rate equating to 600 to 8001/ha, with addition of 0.2% wetting agent. After the test plants have been left to stand in the greenhouse under optimal growth conditions for about 3 weeks, the action of the preparations is assessed visually in comparison to untreated controls (herbicidal action in percent (%): 100% activity=the plants have died, 0% activity=like control plants). Here, numerous compounds according to the invention showed, at an application rate of 80 g or less per hectare, an activity of at least 80% against a large number of important harmful plants. At the same time, inventive compounds leave Gramineae crops such as barley, wheat, rye, millet/sorghum, corn or rice virtually undamaged when applied post-emergence, even at high active compound dosages. In addition, some substances are also harmless to dicotyledonous crops such as soya, cotton, oilseed rape, sugar beet or potatoes. Some of the compounds according to the invention have high selectivity and are therefore suitable for controlling unwanted vegetation in agricultural crops by the post-emergence method. The data of Tables C and D below illustrate, in an exemplary manner, the pre-emergence herbicidal action of the compounds according to the invention, the herbicidal activity being stated in percent.

TABLE C
Post-emergence

| Example | | | | | |
|---|---|---|---|---|---|
| No. | Dosage [g/ha] | ALOMY | AVEFA | ECHCG | SETVI |
| 1-1 | 80 | 90 | 100 | 100 | 100 |
| 1-2 | 80 | 90 | 100 | 90 | 90 |
| 1-3 | 80 | 90 | 100 | 90 | 90 |
| 1-4 | 80 | 90 | 100 | 100 | 100 |
| 1-5 | 80 | 100 | 100 | 100 | 100 |
| 1-6 | 80 | 100 | 100 | 100 | 100 |
| 1-13 | 80 | 90 | 100 | 90 | 90 |
| 1-14 | 80 | 90 | 90 | 100 | 100 |
| 1-15 | 80 | 90 | 100 | 90 | 80 |
| 1-16 | 80 | 90 | 90 | 90 | 90 |
| 1-17 | 80 | 90 | 90 | 90 | 90 |
| 1-18 | 80 | 90 | 100 | 90 | 100 |
| 1-25 | 80 | 100 | 100 | 100 | 100 |
| 1-26 | 80 | 90 | 100 | 100 | 100 |
| 1-27 | 80 | 100 | 100 | 100 | 100 |
| 1-28 | 80 | 90 | 100 | 100 | 100 |
| 1-29 | 80 | 100 | 90 | 100 | 100 |
| 1-30 | 80 | 100 | 100 | 100 | 100 |
| 1-37 | 80 | 80 | 100 | 100 | 100 |
| 1-38 | 80 | 90 | 90 | 100 | 100 |
| 1-39 | 80 | 90 | 80 | 90 | 100 |
| 1-40 | 80 | 90 | 80 | 100 | 100 |
| 1-41 | 80 | 90 | 100 | 90 | 90 |
| 1-42 | 80 | | | 80 | 90 |
| 1-49 | 80 | 90 | 100 | 80 | 90 |
| 1-50 | 80 | 100 | 100 | 100 | 100 |
| 1-51 | 80 | 100 | 100 | 100 | 100 |
| 1-52 | 80 | 100 | 100 | 90 | 90 |
| 1-53 | 80 | 100 | 100 | 90 | 100 |
| 1-54 | 80 | 90 | 100 | 90 | 100 |
| 1-61 | 80 | 90 | 100 | 90 | 90 |
| 1-62 | 80 | 100 | 100 | 100 | 100 |
| 1-63 | 80 | 90 | 100 | 90 | 100 |
| 1-64 | 80 | 90 | 100 | 90 | 90 |
| 1-65 | 80 | 100 | 100 | 100 | 100 |
| 1-74 | 80 | 90 | 90 | 90 | 100 |
| 1-78 | 80 | 90 | 80 | 100 | 100 |
| 2-1 | 80 | 90 | 100 | 90 | 90 |
| 2-2 | 80 | 90 | 90 | 90 | 100 |
| 2-3 | 80 | 80 | 90 | 90 | 100 |
| 2-4 | 80 | 90 | 100 | 90 | 90 |
| 2-5 | 80 | 90 | 100 | 100 | 100 |
| 2-6 | 80 | 80 | 100 | 90 | 90 |
| 2-13 | 80 | 80 | 90 | 90 | 100 |
| 2-14 | 80 | | 90 | 100 | 100 |
| 2-15 | 80 | 80 | 100 | 90 | 100 |
| 2-16 | 80 | 90 | 90 | 90 | 90 |
| 2-17 | 80 | 90 | 100 | 90 | 90 |
| 2-18 | 80 | 80 | 100 | 90 | 90 |
| 2-25 | 80 | 100 | 100 | 100 | 100 |
| 2-26 | 80 | 90 | 100 | 100 | 100 |
| 2-27 | 80 | 90 | 100 | 100 | 100 |
| 2-28 | 80 | 90 | 100 | 100 | 100 |
| 2-29 | 80 | 100 | 90 | 100 | 100 |
| 2-30 | 80 | 90 | 90 | 100 | 100 |
| 2-37 | 80 | 80 | 90 | 100 | 100 |
| 2-38 | 80 | 90 | 90 | 100 | 100 |
| 2-39 | 80 | | 80 | 90 | 100 |
| 2-40 | 80 | 80 | | 100 | 100 |
| 2-41 | 80 | 80 | 90 | 100 | 100 |
| 2-42 | 80 | | | 100 | 100 |
| 2-49 | 80 | 100 | 100 | 100 | 100 |
| 2-50 | 80 | | 100 | 90 | 100 |
| 2-51 | 80 | | 90 | 90 | 90 |
| 2-61 | 80 | 90 | 90 | 90 | 90 |
| 2-62 | 80 | 90 | 100 | 100 | 100 |
| 2-63 | 80 | 100 | 100 | 100 | 100 |
| 2-64 | 80 | 100 | 100 | 100 | 100 |
| 2-78 | 80 | 80 | 90 | 90 | 90 |
| 3-1 | 80 | 90 | 100 | 100 | 100 |
| 3-2 | 80 | 90 | 90 | 90 | 100 |
| 3-3 | 80 | 90 | 90 | 90 | 90 |
| 4-1 | 80 | 80 | | 90 | 100 |
| 4-2 | 80 | | 80 | 100 | 80 |
| 4-3 | 80 | | 90 | 100 | 100 |
| 4-5 | 80 | | | 90 | 90 |
| 4-4 | 80 | | | 100 | 90 |
| 4-6 | 80 | | | 100 | 100 |
| 4-37 | 80 | | | 80 | 90 |
| 4-38 | 80 | | | 80 | |
| 4-40 | 80 | | | 90 | 90 |
| 4-41 | 80 | | | 80 | |
| 4-52 | 80 | 90 | 100 | 100 | 100 |
| 4-53 | 80 | 90 | 100 | 90 | 100 |
| 4-54 | 80 | 90 | 100 | 100 | 100 |
| 4-61 | 80 | 100 | 100 | 100 | 100 |
| 4-64 | 80 | 100 | 100 | 100 | 100 |
| 6-4 | 80 | | | 100 | 100 |
| 6-5 | 80 | | | 100 | 90 |
| 6-6 | 80 | 80 | 80 | 90 | 90 |
| 7-1 | 80 | 90 | | 100 | 100 |
| 7-2 | 80 | | 90 | 90 | 100 |
| 7-3 | 80 | 100 | 100 | 90 | 100 |
| 7-4 | 80 | 90 | 90 | 100 | 100 |
| 7-11 | 80 | 90 | | 90 | 90 |
| 7-12 | 80 | 90 | 90 | 100 | 90 |
| 7-13 | 80 | 90 | 100 | 100 | 100 |
| 7-14 | 80 | 90 | 100 | 90 | 90 |
| 7-15 | 80 | 90 | 100 | 90 | 100 |
| 7-16 | 80 | 90 | 90 | 90 | 100 |
| 7-47 | 80 | 80 | 100 | 100 | 100 |
| 7-50 | 80 | 90 | 90 | 100 | 100 |
| 7-62 | 80 | 90 | 100 | 100 | 100 |
| 7-64 | 80 | 90 | 100 | 100 | 100 |
| 7-75 | 80 | 80 | 100 | 100 | 90 |

TABLE D
Post-emergence

| Example | Dosage | | | | | |
|---|---|---|---|---|---|---|
| No. | [g/ha] | ABUTH | AMARE | PHBPU | STEME | VIOTR |
| 1-1 | 80 | 100 | 100 | 90 | 100 | 100 |
| 1-2 | 80 | 80 | 80 | 90 | 100 | 100 |
| 1-3 | 80 | 80 | | 100 | 100 | 100 |
| 1-4 | 80 | 100 | 100 | 100 | 100 | 100 |
| 1-5 | 80 | 90 | 100 | 100 | 100 | 100 |
| 1-6 | 80 | 90 | 100 | 100 | 100 | 100 |
| 1-13 | 80 | 100 | 100 | 100 | 100 | 100 |
| 1-14 | 80 | 100 | 90 | 90 | 100 | 90 |
| 1-15 | 80 | 90 | 100 | 90 | 100 | 100 |
| 1-16 | 80 | 100 | 100 | | 100 | 100 |
| 1-17 | 80 | 100 | 100 | 90 | 100 | 100 |
| 1-18 | 80 | 100 | 100 | 90 | 100 | 100 |
| 1-25 | 80 | 100 | 100 | 100 | 100 | 100 |
| 1-26 | 80 | 100 | 100 | 90 | 100 | 100 |
| 1-27 | 80 | 100 | 100 | 90 | 100 | 100 |
| 1-28 | 80 | 100 | 100 | 100 | 100 | 100 |
| 1-29 | 80 | 100 | 100 | 100 | 100 | 100 |
| 1-30 | 80 | 100 | 90 | 90 | 100 | 100 |
| 1-37 | 80 | 90 | 100 | 90 | 100 | 100 |
| 1-38 | 80 | 100 | 90 | 90 | 100 | 100 |
| 1-39 | 80 | 90 | 100 | 100 | 100 | 100 |
| 1-40 | 80 | 100 | 100 | 100 | 90 | 90 |
| 1-41 | 80 | 100 | 100 | 90 | 100 | 100 |
| 1-42 | 80 | 90 | 100 | 100 | 100 | 100 |
| 1-49 | 80 | 90 | 80 | 90 | 100 | 100 |
| 1-50 | 80 | 100 | 100 | 100 | 100 | 100 |
| 1-51 | 80 | 100 | 100 | 100 | 100 | 100 |
| 1-52 | 80 | 100 | 100 | 100 | 100 | 100 |
| 1-53 | 80 | 100 | 100 | 100 | 100 | 100 |
| 1-54 | 80 | 100 | 100 | 100 | 100 | 100 |
| 1-61 | 80 | 100 | 90 | 100 | 100 | 100 |
| 1-62 | 80 | 100 | 100 | 100 | 100 | 100 |
| 1-63 | 80 | 100 | 100 | 100 | 100 | 100 |
| 1-64 | 80 | 100 | 100 | 100 | 100 | 100 |
| 1-65 | 80 | 100 | 100 | 100 | 100 | 100 |
| 1-74 | 80 | 100 | 100 | 100 | 100 | 100 |
| 1-75 | 80 | | 90 | 80 | 100 | |
| 1-78 | 80 | 80 | 100 | 100 | 100 | 90 |
| 2-1 | 80 | 90 | 100 | 100 | 100 | 100 |
| 2-2 | 80 | 90 | 80 | 90 | 100 | 100 |
| 2-3 | 80 | 80 | 80 | 100 | 100 | 100 |
| 2-4 | 80 | | | 100 | 100 | 100 |
| 2-5 | 80 | 100 | 100 | 90 | 100 | 100 |
| 2-6 | 80 | 100 | 90 | 100 | 100 | 100 |
| 2-13 | 80 | 100 | 100 | 100 | 100 | 100 |
| 2-14 | 80 | 100 | 100 | 90 | 100 | 100 |
| 2-15 | 80 | 100 | 100 | 80 | 100 | 100 |
| 2-16 | 80 | 100 | 100 | 100 | 100 | 100 |
| 2-17 | 80 | 100 | 100 | 100 | 100 | 100 |
| 2-18 | 80 | 100 | 100 | 100 | 100 | 100 |
| 2-25 | 80 | 100 | 100 | 100 | 100 | 100 |
| 2-26 | 80 | 90 | 100 | 90 | 100 | 100 |
| 2-27 | 80 | 100 | 100 | 90 | 100 | 100 |
| 2-28 | 80 | 100 | 100 | 100 | 100 | 100 |
| 2-29 | 80 | 100 | 100 | 90 | 100 | 100 |
| 2-30 | 80 | 100 | 100 | 90 | 100 | 100 |
| 2-37 | 80 | 100 | 100 | 100 | 100 | 90 |
| 2-38 | 80 | 90 | 100 | 90 | 100 | 100 |
| 2-39 | 80 | 100 | 90 | 90 | 100 | 100 |
| 2-40 | 80 | 100 | 100 | 90 | 100 | 100 |
| 2-41 | 80 | 100 | 90 | 90 | 100 | 100 |
| 2-42 | 80 | 100 | 90 | 90 | 100 | 100 |
| 2-49 | 80 | 100 | 100 | 100 | 100 | 100 |
| 2-50 | 80 | 100 | 100 | 100 | 100 | 100 |
| 2-51 | 80 | 100 | 100 | 90 | 100 | 100 |
| 2-61 | 80 | 100 | 90 | 100 | 100 | 100 |
| 2-62 | 80 | 100 | 100 | 100 | 100 | 100 |
| 2-63 | 80 | 100 | 100 | 100 | 100 | 100 |
| 2-64 | 80 | 100 | 100 | 100 | 100 | 100 |
| 2-78 | 80 | 80 | 100 | 90 | 100 | 90 |
| 3-1 | 80 | 100 | 100 | 90 | 100 | 100 |
| 3-2 | 80 | 80 | | 90 | 100 | 100 |
| 3-3 | 80 | 90 | 80 | 90 | 90 | 100 |
| 4-1 | 80 | 100 | 100 | 100 | 100 | 100 |
| 4-2 | 80 | 90 | 100 | 90 | 90 | 100 |
| 4-3 | 80 | 100 | 100 | 100 | 90 | 100 |
| 4-5 | 80 | 80 | 90 | 90 | 90 | 100 |
| 4-4 | 80 | 80 | 100 | 80 | 90 | 100 |
| 4-6 | 80 | 100 | 100 | 80 | 100 | 100 |
| 4-25 | 80 | | 90 | 80 | 100 | 90 |
| 4-28 | 80 | | 80 | 90 | 100 | 80 |
| 4-29 | 80 | 80 | 80 | 90 | 90 | 90 |
| 4-30 | 80 | 80 | | 90 | 90 | 80 |
| 4-37 | 80 | 80 | 100 | | 80 | 80 |
| 4-38 | 80 | 90 | 90 | 90 | 90 | 100 |
| 4-39 | 80 | 90 | 90 | 90 | 100 | 90 |
| 4-40 | 80 | 80 | 100 | | 90 | 80 |
| 4-41 | 80 | 100 | 90 | 90 | 90 | 90 |
| 4-52 | 80 | 90 | 100 | 100 | 90 | 90 |
| 4-53 | 80 | 100 | 100 | 90 | 90 | 100 |
| 4-54 | 80 | 90 | 90 | 100 | 100 | 100 |
| 4-61 | 80 | 100 | 100 | 100 | 100 | 100 |
| 4-64 | 80 | 100 | 100 | 100 | 100 | 100 |
| 4-75 | 80 | | 100 | 80 | 90 | 80 |
| 5-1 | 80 | 100 | 80 | 90 | 100 | 100 |
| 6-4 | 80 | 90 | | 80 | 90 | 100 |
| 6-5 | 80 | 80 | | | 90 | 100 |
| 6-6 | 80 | 80 | 80 | 100 | 100 | 100 |
| 7-1 | 80 | 100 | 100 | 90 | 90 | 100 |
| 7-2 | 80 | 100 | 100 | 90 | 100 | 100 |
| 7-3 | 80 | 100 | 90 | 90 | 100 | 100 |
| 7-4 | 80 | | 100 | | 90 | 100 |
| 7-11 | 80 | 90 | 90 | 90 | 90 | 100 |
| 7-12 | 80 | 100 | 100 | 90 | 100 | 100 |
| 7-13 | 80 | 100 | 100 | 100 | 100 | 100 |
| 7-14 | 80 | 100 | 100 | 90 | 100 | 100 |
| 7-15 | 80 | 100 | 100 | 90 | 100 | 100 |
| 7-16 | 80 | 100 | 100 | 90 | 100 | 100 |
| 7-47 | 80 | 90 | 90 | 90 | 100 | 100 |
| 7-50 | 80 | 100 | 90 | | 90 | 100 |
| 7-62 | 80 | 100 | 100 | 90 | 100 | 100 |
| 7-64 | 80 | 100 | 100 | 100 | 90 | 90 |
| 7-75 | 80 | 90 | 100 | 80 | 80 | 100 |

### 3. Comparative Experiments

For comparison, the herbicidal activity of numerous compounds according to the invention was tested with the structurally closest compounds known from the documents WO 2011/035874, WO 2012/028579 and WO 2012/126932, by the pre- and post-emergence method. These data are listed in Tables E to M below, where in each comparison pair the first compound is the compound according to the invention and the second compound is the compound known from the prior art.

TABLE E
Comparison with compounds known from WO 2011035874,
applied by the post-emergence method

| Example | Dosage | Herbicidal efficacy against | | | | | | |
|---|---|---|---|---|---|---|---|---|
| No. | [g/ha] | ABUTH | AMARE | MATIN | PHBPU | STEME | VIOTR | VERPE |
| 5-1 | 80 | 100 | 80 | 40 | 90 | 100 | 100 | 100 |
| 1-81 | 80 | 30 | 60 | 10 | 10 | 0 | 0 | 30 |

TABLE F
Comparison with compounds known from WO2011035874,
applied by the post-emergence method

| Example | Dosage | Herbicidal efficacy against | | | | |
|---|---|---|---|---|---|---|
| No. | [g/ha] | ABUTH | MATIN | PHBPU | STEME | VIOTR |
| 5-1 | 80 | 100 | | | 100 | |
| 1-51 | 80 | 80 | | | 40 | |
| 5-1 | 80 | | 40 | 90 | 100 | 100 |
| 1-77 | 80 | | 0 | 40 | 80 | 60 |

TABLE G
Comparison with compounds known from WO 2011035874,
applied by the pre-emergence method

| Example | Dosage | Herbicidal efficacy against | | | | | |
|---|---|---|---|---|---|---|---|
| No. | [g/ha] | ALOMY | AVEFA | CYPES | ECHCG | SETVI | ABUTH |
| 5-1 | 80 | 30 | 30 | 50 | 80 | 80 | 100 |
| 1-51 | 80 | 10 | 0 | 0 | 30 | 60 | 100 |
| 5-1 | 80 | 30 | 30 | 50 | 80 | 80 | 100 |
| 1-81 | 80 | 0 | 40 | 0 | 10 | 10 | 20 |
| 5-1 | 320 | 40 | 60 | 80 | 100 | 100 | 100 |
| 1-77 | 320 | 10 | 0 | 30 | 70 | 20 | 100 |

TABLE H
Comparison with compounds known from WO 2011035874,
applied by the pre-emergence method

| Example | Dosage | Herbicidal efficacy against | | | | |
|---|---|---|---|---|---|---|
| No. | [g/ha] | AMARE | POLCO | STEME | VIOTR | VERPE |
| 5-1 | 80 | 100 | 80 | 80 | 100 | 100 |
| 1-51 | 80 | 100 | 0 | 90 | 60 | 100 |
| 5-1 | 80 | 100 | 80 | 80 | 100 | 100 |
| 1-81 | 80 | 60 | 10 | 50 | 90 | 70 |
| 5-1 | 320 | 100 | 100 | 90 | 100 | 100 |
| 1-77 | 320 | 100 | 30 | 80 | 60 | 90 |

TABLE I
Comparison with compounds known from WO 2012/028579,
applied by the post-emergence method

| Example | Dosage | Herbicidal efficacy against | | | | | |
|---|---|---|---|---|---|---|---|
| No. | [g/ha] | ALOMY | AVEFA | CYPES | SETVI | STEME | VIOTR |
| 1-1 | 80 | 90 | 100 | 80 | 100 | 100 | 100 |
| 4-803 | 80 | 20 | 0 | 60 | 100 | 100 | 100 |
| 1-2 | 5 | 60 | 70 | 50 | 90 | 90 | 100 |
| 4-804 | 5 | 0 | 0 | 0 | 10 | 10 | 20 |
| 1-3 | 80 | 90 | 100 | 80 | 90 | 100 | 100 |
| 4-805 | 80 | 0 | 0 | 0 | 0 | 0 | 0 |
| 1-1 | 20 | 90 | 90 | 40 | 100 | 100 | 100 |
| 4-812 | 20 | 30 | 50 | 40 | 100 | 100 | 90 |
| 1-2 | 5 | 60 | 70 | | | | 100 |
| 4-813 | 5 | 30 | 40 | | | | 100 |
| 1-3 | 5 | 60 | 70 | 60 | 90 | 90 | 100 |
| 4-814 | 5 | 0 | 30 | 20 | 80 | 90 | 100 |
| 1-49 | 5 | 60 | 50 | 50 | 80 | | 100 |
| 4-803 | 5 | 0 | 0 | 20 | 0 | | 90 |
| 1-51 | 20 | 90 | 90 | 90 | 100 | 100 | 100 |
| 4-805 | 20 | 0 | 0 | 0 | 0 | 0 | 0 |
| 1-49 | 5 | 60 | 50 | 50 | | | 100 |
| 4-812 | 5 | 10 | 20 | 20 | | | 70 |
| 1-51 | 5 | 40 | 60 | 80 | | 100 | 100 |
| 4-814 | 5 | 0 | 30 | 20 | | 90 | 100 |
| 1-2 | 5 | 60 | 70 | 50 | 90 | 90 | 100 |
| 4-136 | 5 | 40 | 80 | 80 | 90 | 100 | 80 |
| 1-2 | 5 | 60 | 70 | 50 | 90 | 90 | 100 |
| 4-175 | 5 | 0 | 0 | 30 | 40 | 80 | 70 |
| 1-3 | 5 | 60 | 70 | | | | 100 |
| 4-176 | 5 | 30 | 10 | | | | 100 |
| 1-1 | 20 | 90 | 90 | 40 | 100 | 100 | 100 |
| 4-78 | 20 | 20 | 80 | 40 | 80 | 80 | 60 |
| 1-2 | 20 | 80 | 90 | 70 | 90 | 90 | 100 |
| 4-79 | 20 | 20 | 40 | 10 | 90 | 40 | 40 |
| 1-3 | 5 | 60 | | 60 | 90 | 90 | 100 |
| 4-80 | 5 | 10 | | 20 | 20 | 90 | 60 |
| 1-1 | 5 | 60 | | | 100 | 100 | 100 |
| 4-108 | 5 | 60 | | | 90 | 70 | 90 |
| 1-2 | 5 | 60 | 70 | 50 | 90 | 90 | |
| 4-109 | 5 | 20 | 40 | 40 | 90 | 90 | |
| 1-3 | 5 | 60 | 70 | | 90 | 90 | 100 |
| 4-110 | 5 | 20 | 20 | | 90 | 90 | 100 |
| 1-1 | 5 | 60 | 40 | | 100 | 100 | 100 |
| 4-128 | 5 | 20 | 0 | | 80 | 60 | 100 |
| 1-2 | 5 | 60 | 70 | 50 | 90 | 90 | 100 |
| 4-129 | 5 | 20 | 0 | 50 | 80 | 70 | 80 |
| 1-3 | 5 | 60 | 70 | 60 | 90 | 90 | 100 |
| 4-130 | 5 | 20 | 0 | 10 | 60 | 100 | 90 |
| 1-1 | 5 | 60 | 40 | 20 | 100 | 100 | 100 |
| 4-122 | 5 | 80 | 40 | 20 | 40 | 90 | 100 |
| 1-2 | 5 | 60 | 70 | 50 | | 90 | 100 |
| 4-123 | 5 | 30 | 10 | 20 | | 90 | 100 |
| 1-3 | 5 | 60 | 70 | 60 | 90 | 90 | 100 |
| 4-124 | 5 | 20 | 60 | 60 | 90 | 70 | 100 |
| 1-1 | 5 | 60 | 40 | | 100 | 100 | 100 |
| 4-406 | 5 | 60 | 0 | | 80 | 100 | 100 |
| 1-49 | 5 | 60 | | 50 | 80 | 70 | 100 |
| 4-229 | 5 | 10 | | 10 | 90 | 40 | 40 |
| 1-51 | 5 | 40 | | 80 | 90 | 100 | 100 |
| 4-231 | 5 | 0 | | 50 | 90 | 90 | 60 |
| 1-49 | 5 | 60 | 50 | | 80 | | 100 |
| 4-292 | 5 | 10 | 0 | | 40 | | 70 |
| 1-51 | 5 | 40 | 60 | 80 | | 100 | 100 |
| 4-756 | 5 | 40 | 0 | 10 | | 100 | 90 |
| 1-49 | 5 | 60 | 50 | 50 | | | 100 |
| 4-245 | 5 | 30 | 20 | 30 | | | 90 |
| 1-51 | 80 | 100 | 100 | 100 | 100 | 100 | 100 |
| 4-634 | 80 | 80 | 80 | 100 | 100 | 100 | 100 |
| 1-51 | 20 | 90 | 90 | 90 | 100 | 100 | 100 |
| 4-640 | 20 | 50 | 70 | 60 | 100 | 100 | 100 |
| 4-1 | 20 | 70 | 20 | 0 | 60 | 80 | 100 |
| 1-573 | 20 | 0 | 0 | 10 | 10 | 10 | 70 |
| 4-3 | 20 | 30 | 50 | 70 | 40 | 60 | 100 |
| 1-574 | 20 | 0 | 0 | 0 | 20 | 50 | 80 |
| 4-3 | 20 | | 50 | 70 | 40 | 60 | 100 |
| 1-90 | 20 | | 0 | 0 | 0 | 10 | 0 |
| 4-1 | 20 | 70 | 20 | 0 | 60 | 80 | 100 |
| 1-119 | 20 | 0 | 0 | 0 | 10 | 40 | 60 |
| 4-3 | 20 | | 50 | 70 | 40 | | 100 |
| 1-121 | 20 | | 0 | 40 | 40 | | 40 |
| 4-1 | 20 | 70 | 20 | 0 | 60 | 80 | 100 |
| 1-387 | 20 | 10 | 0 | 0 | 0 | 60 | 60 |
| 4-1 | 20 | 70 | 20 | 0 | 60 | 80 | 100 |
| 1-139 | 20 | 0 | 0 | 0 | 0 | 40 | 80 |

TABLE J
Comparison with compounds known from WO 2012/028579,
applied by the pre-emergence method

| Example | Dosage | Herbicidal efficacy against | | | | |
|---|---|---|---|---|---|---|
| No. | [g/ha] | ALOMY | AVEFA | CYPES | ECHCG | SETVI |
| 1-1 | 20 | 90 | | 30 | 80 | 70 |
| 4-803 | 20 | 0 | | 0 | 0 | 0 |
| 1-2 | 320 | 100 | 100 | 90 | 100 | 100 |
| 4-804 | 320 | 0 | 10 | 10 | 0 | 0 |
| 1-2 | 80 | 90 | 70 | 80 | 100 | 90 |
| 4-804 | 80 | 0 | 0 | 10 | 0 | 0 |
| 1-3 | 320 | 100 | 100 | 90 | 100 | 100 |
| 4-805 | 320 | 0 | 0 | 0 | 0 | 0 |
| 1-3 | 80 | 90 | 100 | 90 | 100 | 100 |
| 4-805 | 80 | 0 | 0 | 0 | 0 | 0 |
| 1-1 | 20 | 90 | | 30 | | 70 |
| 4-812 | 20 | 50 | | 0 | | 40 |
| 1-3 | 80 | 90 | 100 | 90 | | 100 |
| 4-814 | 80 | 40 | 20 | 20 | | 20 |
| 1-3 | 20 | 50 | 80 | 30 | | 70 |
| 4-814 | 20 | 20 | 10 | 10 | | 10 |
| 1-49 | 20 | 40 | 50 | 50 | 50 | 90 |
| 4-803 | 20 | 0 | 0 | 0 | 0 | 0 |
| 1-50 | 320 | 100 | 100 | 100 | 100 | 100 |
| 4-804 | 320 | 0 | 10 | 10 | 0 | 0 |
| 1-50 | 80 | 100 | 90 | 100 | 100 | 100 |
| 4-804 | 80 | 0 | 0 | 10 | 0 | 0 |
| 1-51 | 320 | 100 | 100 | 100 | 100 | 100 |
| 4-805 | 320 | 0 | 0 | 0 | 0 | 0 |
| 1-51 | 80 | 100 | 90 | 90 | 100 | 100 |
| 4-805 | 80 | 0 | 0 | 0 | 0 | 0 |
| 1-50 | 20 | 90 | | | | 100 |
| 4-813 | 20 | 0 | | | | 10 |
| 1-51 | 80 | 100 | 90 | 90 | | 100 |
| 4-814 | 80 | 40 | 20 | 20 | | 20 |
| 1-51 | 20 | 80 | | 90 | | 100 |
| 4-814 | 20 | 20 | | 10 | | 10 |
| 1-1 | 20 | 90 | | 30 | | |
| 4-135 | 20 | 60 | | 0 | | |
| 1-3 | 20 | | 80 | | 90 | |
| 4-137 | 20 | | 0 | | 20 | |
| 1-1 | 20 | 90 | | 30 | 80 | 70 |
| 4-174 | 20 | 0 | | 10 | 0 | 0 |
| 1-2 | 20 | 70 | 20 | 60 | 40 | |
| 4-175 | 20 | 20 | 0 | 0 | 0 | |
| 1-3 | 20 | 50 | 80 | | 90 | 70 |
| 4-176 | 20 | 20 | 20 | | 20 | 40 |
| 1-1 | 80 | 90 | 90 | 70 | 100 | 100 |
| 4-78 | 80 | 10 | 30 | 10 | 0 | 70 |
| 1-2 | 80 | 90 | 70 | 80 | 100 | 90 |
| 4-79 | 80 | 20 | 20 | 0 | 0 | 20 |
| 1-3 | 20 | 50 | 80 | 30 | 90 | 70 |
| 4-80 | 20 | 0 | 10 | 0 | 0 | 0 |
| 1-1 | 20 | 90 | | 30 | | |
| 4-108 | 20 | 50 | | 0 | | |
| 1-2 | 20 | | | 60 | | |
| 4-109 | 20 | | | 0 | | |
| 1-3 | 20 | | 80 | | | 70 |
| 4-110 | 20 | | 10 | | | 20 |
| 1-1 | 20 | 90 | | 30 | 80 | 70 |
| 4-128 | 20 | 10 | | 0 | 0 | 10 |
| 1-2 | 80 | 90 | 70 | | | |
| 4-129 | 80 | 30 | 50 | | | |
| 1-3 | 20 | 50 | 80 | 30 | 90 | 70 |
| 4-130 | 20 | 20 | 0 | 0 | 70 | 20 |
| 1-1 | 20 | | | 30 | 80 | |
| 4-122 | 20 | | | 0 | 50 | |
| 1-2 | 20 | 70 | 20 | 60 | | |
| 4-123 | 20 | 20 | 0 | 0 | | |
| 1-3 | 20 | 50 | 80 | 30 | | |
| 4-124 | 20 | 10 | 0 | 10 | | |
| 1-1 | 20 | 90 | | | 80 | 70 |
| 4-406 | 20 | 0 | | | 10 | 0 |
| 1-49 | 20 | 40 | 50 | 50 | 50 | 90 |
| 4-229 | 20 | 0 | 0 | 0 | 10 | 50 |
| 1-50 | 80 | 100 | | 100 | | |
| 4-230 | 80 | 60 | | 40 | | |
| 1-50 | 20 | 90 | | 70 | 90 | 100 |
| 4-230 | 20 | 40 | | 0 | 0 | 60 |
| 1-51 | 20 | 80 | | 90 | 100 | |
| 4-231 | 20 | 20 | | 50 | 70 | |
| 1-49 | 20 | 40 | 50 | | | 90 |
| 4-292 | 20 | 20 | 0 | | | 20 |
| 1-50 | 80 | 100 | 90 | 100 | 100 | 100 |
| 4-293 | 80 | 20 | 0 | 40 | 0 | 10 |
| 1-51 | 20 | 80 | | 90 | 100 | 100 |
| 4-756 | 20 | 40 | | 0 | 0 | 40 |
| 1-49 | 80 | | 80 | 70 | | |
| 4-245 | 80 | | 60 | 30 | | |
| 1-49 | 20 | | | 50 | | |
| 4-245 | 20 | | | 0 | | |
| 1-50 | 20 | 90 | | 70 | 90 | 100 |
| 4-246 | 20 | 70 | | 50 | 60 | 70 |
| 1-51 | 20 | 80 | | 90 | | |
| 4-247 | 20 | 60 | | 70 | | |
| 1-49 | 80 | 90 | 80 | 70 | 100 | |
| 4-632 | 80 | 30 | 0 | 20 | 70 | |
| 1-50 | 20 | 90 | | 70 | | 100 |
| 4-633 | 20 | 0 | | 20 | | 10 |
| 1-51 | 20 | 80 | | 90 | 100 | 100 |
| 4-634 | 20 | 60 | | 60 | 80 | 30 |
| 1-49 | 20 | | 50 | 50 | | |
| 4-638 | 20 | | 0 | 0 | | |
| 1-50 | 20 | 90 | | | | 100 |
| 4-639 | 20 | 30 | | | | 70 |
| 1-51 | 20 | | | 90 | | |
| 4-640 | 20 | | | 0 | | |
| 4-1 | 320 | 40 | 80 | 50 | 100 | 100 |
| 1-573 | 320 | 0 | 0 | 0 | 60 | 0 |
| 4-3 | 320 | 90 | 80 | 100 | 100 | 100 |
| 1-574 | 320 | 0 | 0 | 50 | 30 | 0 |
| 4-1 | 80 | | 30 | 30 | 70 | 80 |
| 1-146 | 80 | | 0 | 0 | 30 | 0 |
| 4-3 | 80 | 60 | 30 | 100 | 100 | 90 |
| 1-148 | 80 | 0 | 0 | 0 | 0 | 0 |
| 4-1 | 80 | | 30 | 30 | 70 | 80 |
| 1-186 | 80 | | 0 | 0 | 0 | 0 |
| 4-3 | 80 | 60 | 30 | 100 | 100 | 90 |
| 1-188 | 80 | 10 | 0 | 0 | 0 | 0 |
| 4-3 | 320 | 90 | 80 | | 100 | 100 |
| 1-90 | 320 | 0 | 10 | | 30 | 20 |
| 4-1 | 320 | 40 | 80 | 50 | 100 | 100 |
| 1-119 | 320 | 0 | 40 | 0 | 20 | 40 |
| 4-3 | 80 | 60 | 30 | 100 | 100 | 90 |
| 1-121 | 80 | 0 | 0 | 20 | 0 | 0 |
| 4-1 | 80 | | 30 | 30 | 70 | 80 |
| 1-387 | 80 | | 0 | 0 | 0 | 0 |
| 4-3 | 80 | 60 | 30 | 100 | 100 | 90 |
| 1-389 | 80 | 30 | 0 | 0 | 0 | 0 |
| 4-1 | 320 | 40 | 80 | | 100 | 100 |
| 1-139 | 320 | 0 | 30 | | 20 | 0 |

TABLE K
Comparison with compounds known from WO 2012/028579,
applied by the pre-emergence method

| | Dosage | Herbicidal efficacy against | | | |
|---|---|---|---|---|---|
| Example No. | [g/ha] | ABUTH | MATIN | POLCO | VIOTR |
| 1-1 | 20 | 90 | 70 | 60 | 100 |
| 4-803 | 20 | 0 | 10 | 0 | 0 |
| 1-2 | 320 | 100 | 100 | 80 | 100 |
| 4-804 | 320 | 80 | 0 | 0 | 0 |
| 1-2 | 80 | 100 | 100 | 70 | 100 |
| 4-804 | 80 | 0 | 0 | 0 | 0 |
| 1-3 | 320 | 100 | 100 | 90 | 100 |
| 4-805 | 320 | 20 | 30 | 0 | 0 |
| 1-3 | 80 | 100 | 100 | 90 | 100 |
| 4-805 | 80 | 0 | 30 | 0 | 0 |
| 1-3 | 80 | | 100 | 90 | |
| 4-814 | 80 | | 80 | 20 | |
| 1-3 | 20 | 100 | 80 | 30 | 100 |
| 4-814 | 20 | 40 | 20 | 10 | 80 |
| 1-49 | 20 | 70 | 80 | | 100 |
| 4-803 | 20 | 0 | 10 | | 0 |
| 1-50 | 320 | 100 | 100 | 90 | 100 |
| 4-804 | 320 | 80 | 0 | 0 | 0 |
| 1-50 | 80 | 100 | 100 | 80 | 100 |
| 4-804 | 80 | 0 | 0 | 0 | 0 |
| 1-51 | 320 | 100 | 100 | 90 | 100 |
| 4-805 | 320 | 20 | 30 | 0 | 0 |
| 1-51 | 80 | 100 | 100 | 80 | 100 |
| 4-805 | 80 | 0 | 30 | 0 | 0 |
| 1-50 | 20 | | 90 | | |
| 4-813 | 20 | | 70 | | |
| 1-51 | 80 | | 100 | 80 | |
| 4-814 | 80 | | 80 | 20 | |
| 1-51 | 20 | 100 | 90 | 30 | 100 |
| 4-814 | 20 | 40 | 20 | 10 | 80 |
| 1-1 | 20 | | 70 | 60 | 100 |
| 4-135 | 20 | | 0 | 0 | 50 |
| 1-3 | 20 | | | 30 | |
| 4-137 | 20 | | | 0 | |
| 1-1 | 20 | 90 | 70 | 60 | 100 |
| 4-174 | 20 | 70 | 30 | 0 | 70 |
| 1-2 | 20 | | 70 | | 80 |
| 4-175 | 20 | | 40 | | 0 |
| 1-3 | 20 | | | 30 | 100 |
| 4-176 | 20 | | | 0 | 80 |
| 1-1 | 80 | 100 | 90 | 70 | 100 |
| 4-78 | 80 | 50 | 0 | 0 | 10 |
| 1-2 | 80 | 100 | 100 | 70 | 100 |
| 4-79 | 80 | 0 | 0 | 0 | 0 |
| 1-3 | 20 | 100 | 80 | 30 | 100 |
| 4-80 | 20 | 0 | 0 | 0 | 0 |
| 1-1 | 20 | 90 | 70 | | 100 |
| 4-108 | 20 | 60 | 0 | | 20 |
| 1-2 | 20 | | | | 80 |
| 4-109 | 20 | | | | 10 |
| 1-3 | 20 | | | | 100 |
| 4-110 | 20 | | | | 80 |
| 1-1 | 20 | 90 | 70 | 60 | 100 |
| 4-128 | 20 | 70 | 50 | 0 | 50 |
| 1-2 | 80 | | | 70 | |
| 4-129 | 80 | | | 10 | |
| 1-3 | 20 | | 80 | 30 | |
| 4-130 | 20 | | 20 | 0 | |
| 1-1 | 20 | | | 60 | 100 |
| 4-122 | 20 | | | 0 | 70 |
| 1-2 | 20 | | 70 | | 80 |
| 4-123 | 20 | | 0 | | 20 |
| 1-3 | 20 | | | 30 | |
| 4-124 | 20 | | | 0 | |
| 1-1 | 20 | 90 | | 60 | 100 |
| 4-406 | 20 | 40 | | 10 | 0 |
| 1-49 | 20 | 70 | 80 | | 100 |
| 4-229 | 20 | 0 | 0 | | 0 |
| 1-50 | 80 | 100 | 100 | 80 | 100 |
| 4-230 | 80 | 80 | 0 | 0 | 30 |
| 1-50 | 20 | 100 | | | 100 |
| 4-230 | 20 | 30 | | | 0 |
| 1-51 | 20 | 100 | 90 | 30 | 100 |
| 4-231 | 20 | 60 | 70 | 0 | 0 |
| 1-50 | 80 | 100 | | 80 | 100 |
| 4-293 | 80 | 40 | | 0 | 70 |
| 1-51 | 20 | 100 | | 30 | 100 |
| 4-756 | 20 | 70 | | 0 | 20 |
| 1-49 | 20 | | | | 100 |
| 4-245 | 20 | | | | 50 |
| 1-50 | 20 | | | | 100 |
| 4-246 | 20 | | | | 20 |
| 1-49 | 80 | 100 | | 50 | 100 |
| 4-632 | 80 | 60 | | 0 | 70 |
| 1-50 | 20 | 100 | 90 | | 100 |
| 4-633 | 20 | 60 | 60 | | 0 |
| 1-51 | 20 | 100 | 90 | 30 | |
| 4-634 | 20 | 70 | 70 | 0 | |
| 1-49 | 20 | | 80 | | |
| 4-638 | 20 | | 40 | | |
| 1-50 | 20 | | 90 | | 100 |
| 4-639 | 20 | | 60 | | 80 |
| 1-51 | 20 | | | 30 | |
| 4-640 | 20 | | | 0 | |
| 4-1 | 320 | 100 | | 30 | 100 |
| 1-573 | 320 | 60 | | 0 | 80 |
| 4-3 | 320 | 100 | 100 | 70 | 100 |
| 1-574 | 320 | 40 | 70 | 0 | 10 |
| 4-1 | 80 | 90 | 80 | 30 | 100 |
| 1-146 | 80 | 70 | 60 | 0 | 30 |
| 4-3 | 80 | 100 | | 40 | |
| 1-148 | 80 | 0 | | 0 | |
| 4-1 | 80 | 90 | 80 | 30 | 100 |
| 1-186 | 80 | 0 | 0 | 0 | 0 |
| 4-3 | 80 | 100 | 100 | 40 | 100 |
| 1-188 | 80 | 0 | 40 | 0 | 40 |
| 4-3 | 320 | 100 | 100 | 70 | 100 |
| 1-90 | 320 | 0 | 0 | 10 | 0 |
| 4-1 | 320 | 100 | | | 100 |
| 1-119 | 320 | 40 | | | 0 |
| 4-3 | 80 | 100 | | 40 | 100 |
| 1-121 | 80 | 60 | | 0 | 50 |
| 4-1 | 80 | 90 | 80 | 30 | 100 |
| 1-387 | 80 | 30 | 30 | 0 | 0 |
| 4-3 | 80 | 100 | 100 | | 100 |
| 1-389 | 80 | 30 | 50 | | 80 |
| 4-1 | 320 | 100 | 90 | 30 | |
| 1-139 | 320 | 70 | 70 | 0 | |

TABLE L
Comparison with compounds known from WO2012126932,
applied by the pre-emergence method

| Example | Dosage | Herbicidal efficacy against | | | | | | |
|---|---|---|---|---|---|---|---|---|
| No. | [g/ha] | ALOMY | AVEFA | CYPES | LOLMU | SETVI | POLCO | VIOTR |
| 7-1 | 80 | 60 | 20 | 60 | 0 | | 30 | |
| 2-143 | 80 | 30 | 0 | 0 | 0 | | 0 | |
| 7-3 | 80 | 90 | 90 | 100 | 80 | 100 | 40 | 100 |
| 2-145 | 80 | 90 | 80 | 90 | 60 | 100 | 10 | 100 |
| 7-1 | 80 | 60 | 20 | | 0 | 70 | | 90 |
| 2-183 | 80 | 0 | 0 | | 0 | 0 | | 0 |
| 7-3 | 80 | 90 | 90 | 100 | 80 | 100 | 40 | 100 |
| 2-185 | 80 | 80 | 20 | 70 | 40 | 100 | 40 | 100 |
| 7-1 | 320 | 70 | 70 | 70 | 30 | 100 | | 100 |
| 2-130 | 320 | 70 | 30 | 0 | 30 | 100 | | 90 |
| 7-1 | 80 | 60 | 20 | 60 | 0 | 70 | 30 | 90 |
| 2-136 | 80 | 0 | 0 | 0 | 0 | 20 | 0 | 0 |
| 7-1 | 320 | | 70 | 70 | 30 | 100 | | 100 |
| 2-116 | 320 | | 40 | 50 | 10 | 90 | | 80 |
| 7-2 | 80 | 80 | 70 | 80 | 10 | 90 | 30 | 100 |
| 2-117 | 80 | 20 | 0 | 50 | 0 | 20 | 0 | 0 |
| 7-3 | 80 | 90 | 90 | 100 | 80 | 100 | | 100 |
| 2-118 | 80 | 30 | 50 | 70 | 40 | 60 | | 90 |
| 7-1 | 320 | 70 | 70 | 70 | 30 | 100 | 40 | 100 |
| 2-85 | 320 | 0 | 0 | 40 | 0 | 0 | 0 | 0 |
| 7-3 | 320 | 100 | 100 | 100 | 100 | 100 | 70 | 100 |
| 2-87 | 320 | 70 | 30 | 20 | 0 | 80 | 20 | 60 |

TABLE M
Comparison with compounds known from WO2012126932,
applied by the post-emergence method

| Example | Dosage | Herbicidal efficacy against | | | | | |
|---|---|---|---|---|---|---|---|
| No. | [g/ha] | ALOMY | AVEFA | SETVI | MATIN | VIOTR | VERPE |
| 7-1 | 20 | 70 | 40 | 100 | | 100 | 90 |
| 2-143 | 20 | 50 | 20 | 100 | | 60 | 80 |
| 7-2 | 20 | | 70 | 100 | 70 | 100 | 100 |
| 2-144 | 20 | | 70 | 90 | 90 | 60 | 100 |
| 7-1 | 20 | 70 | 40 | 100 | | 100 | |
| 2-183 | 20 | 20 | 10 | 70 | | 70 | |
| 7-1 | 20 | 70 | 40 | 100 | 40 | 100 | 90 |
| 2-130 | 20 | 40 | 20 | 80 | 10 | 30 | 80 |
| 7-1 | 20 | 70 | 40 | 100 | 40 | 100 | 90 |
| 2-136 | 20 | 30 | 0 | 90 | 20 | 30 | 60 |
| 7-1 | 20 | 70 | 40 | 100 | 40 | 100 | 90 |
| 2-116 | 20 | 70 | 40 | 70 | 40 | 60 | 50 |
| 7-2 | 20 | | 70 | 100 | 70 | 100 | 100 |
| 2-117 | 20 | | 60 | 70 | 30 | 90 | 70 |
| 7-3 | 80 | 100 | 100 | 100 | 100 | 100 | 100 |
| 2-118 | 80 | 70 | 100 | 90 | 70 | 100 | 90 |
| 7-3 | 20 | 60 | 90 | 90 | 90 | 100 | 100 |
| 2-118 | 20 | 60 | 90 | 90 | 50 | 90 | 80 |
| 7-1 | 20 | 70 | 40 | 100 | 40 | 100 | 90 |
| 2-85 | 20 | 0 | 0 | 40 | 20 | 0 | 50 |
| 7-3 | 80 | 100 | 100 | 100 | 100 | 100 | 100 |
| 2-87 | 80 | 60 | 70 | 80 | 0 | 70 | 80 |

1. A benzoylamide of formula (I) or a salt thereof 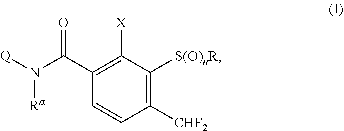
in which the symbols and indices are defined as follows:
Q represents a radical Q1, Q2, Q3 or Q4, 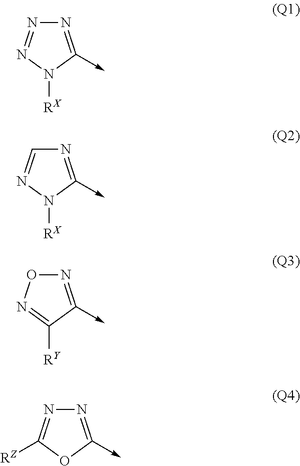
X represents (C₁-C₆)-alkyl, (C₃-C₆)-cycloalkyl or halogen,
R represents (C₁-C₆)-alkyl, (C₃-C₆)-cycloalkyl, (C₃-C₆)-cycloalkyl-(C₁-C₆)-alkyl, (C₁-C₆)-alkyl-O—(C₁-C₆)-alkyl,
R^{a} represents hydrogen, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₆)-cycloalkyl, halo-(C₃-C₆)-cycloalkyl, (C₃-C₆)-cycloalkyl-(C₁-C₆)-alkyl, halo-(C₃-C₆)-cycloalkyl-(C₁-C₆)-alkyl, R¹(O)C—(C₁-C₆)-alkyl, R¹O(O)C—(C₁-C₆)-alkyl, (R¹)₂N(O)C—(C₁-C₆)-alkyl, NC—(C₁-C₆)-alkyl, R¹O—(C₁-C₆)-alkyl, R¹(O)C₀—(C₁-C₆)-alkyl, R²(O)₂SO—(C₁-C₆)-alkyl, (R¹)₂N—(C₁-C₆)-alkyl, R¹(O)C(R¹)N—(C₁-C₆)-alkyl, R²(O)₂S(R¹)N—(C₁-C₆)-alkyl, R²(O)ₙS—(C₁-C₆)-alkyl, R¹O(O)₂S—(C₁-C₆)-alkyl, (R¹)₂N(O)₂S—(C₁-C₆)-alkyl, R¹(O)C, R¹O(O)C, (R¹)₂N(O)C, R¹O, (R¹)₂N, R²O(O)C(R¹)N, (R¹)₂N(O)C(R¹)N, R²(O)₂S,
or benzyl substituted in each case by s radicals from the group consisting of methyl, ethyl, methoxy, nitro, trifluoromethyl and halogen,
R^{X} represents (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, where the six radicals mentioned above are in each case substituted by s radicals from the group consisting of nitro, cyano, (R⁶)₃Si, (R⁵O)₂(O)P, R²(O)ₙS, (R¹)₂N, R¹O, R¹(O)C, R¹O(O)C, R¹(O)CO, R²O(O)CO, R¹(O)C(R¹)N, R²(O)₂S(R¹)N, (C₃-C₆)-cycloalkyl, heteroaryl, heterocyclyl and phenyl, where the four last-mentioned radicals are substituted by s radicals from the group consisting of (C₁-C₆)-alkyl, halo-(C₁-C₆)-alkyl, (C₁-C₆)-alkoxy, halo-(C₁-C₆)-alkoxy and halogen, and where heterocyclyl carries n oxo groups,
or R^{X} represents (C₃-C₇)-cycloalkyl, heteroaryl, heterocyclyl or phenyl, where the four radicals mentioned above are in each case substituted by s radicals from the group consisting of halogen, nitro, cyano, (C₁-C₆)-alkyl, halo-(C₁-C₆)-alkyl, (C₃-C₆)-cycloalkyl, (C₁-C₆)-alkyl-S(O)ₙ, (C₁-C₆)-alkoxy, halo-(C₁-C₆)-alkoxy and (C₁-C₆)-alkoxy-(C₁-C₄)-alkyl,
R^{Y} represents hydrogen, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₇)-cycloalkyl, (C₁-C₆)-alkoxy, halo-(C₁-C₆)-alkoxy, (C₂-C₆)-alkenyloxy, (C₂-C₆)-alkynyloxy, cyano, nitro, methylsulfenyl, methylsulfinyl, methylsulfonyl, acetylamino, benzoylamino, methoxycarbonyl, ethoxycarbonyl, methoxycarbonylmethyl, ethoxycarbonylmethyl, benzoyl, methylcarbonyl, piperidinylcarbonyl, trifluoromethylcarbonyl, halogen, amino, aminocarbonyl, methylaminocarbonyl, dimethylaminocarbonyl, methoxymethyl, or represents heteroaryl, heterocyclyl or phenyl, each of which is substituted by s radicals from the group consisting of (C₁-C₆)-alkyl, halo-(C₁-C₆)-alkyl, (C₁-C₆)-alkoxy, halo-(C₁-C₆)-alkoxy and halogen, and where heterocyclyl carries n oxo groups,
R^{Z} represents hydrogen, (C₁-C₆)-alkyl, R¹O—(C₁-C₆)-alkyl, R′CH₂, (C₃-C₇)-cycloalkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, R¹O, R¹(H)N, methoxycarbonyl, ethoxycarbonyl, methylcarbonyl, dimethylamino, trifluoromethylcarbonyl, acetylamino, methylsulfenyl, methylsulfinyl, methylsulfonyl, or represents heteroaryl, heterocyclyl, benzyl or phenyl, each of which is substituted by s radicals from the group consisting of halogen, nitro, cyano, (C₁-C₆)-alkyl, halo-(C₁-C₆)-alkyl, (C₃-C₆)-cycloalkyl, (C₁-C₆)-alkyl-S(O)ₙ, (C₁-C₆)-alkoxy, halo-(C₁-C₆)-alkoxy and (C₁-C₆)-alkoxy-(C₁-C₄)-alkyl, where heterocyclyl carries n oxo groups,
R¹ represents hydrogen, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₆)-cycloalkyl, (C₃-C₆)-cycloalkenyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, phenyl-N(R³)—(C₁-C₆)-alkyl, heteroaryl-N(R³)—(C₁-C₆)-alkyl, heterocyclyl-N(R³)—(C₁-C₆)-alkyl, phenyl-S(O)ₙ—(C₁-C₆)-alkyl, heteroaryl-S(O)ₙ—(C₁-C₆)-alkyl or heterocyclyl-S(O)ₙ—(C₁-C₆)-alkyl, where the fifteen last-mentioned radicals are in each case substituted by s radicals from the group consisting of nitro, halogen, cyano, thiocyanato, (C₁-C₆)-alkyl, halo-(C₁-C₆)-alkyl, (C₃-C₆)-cycloalkyl, R³O(O)C, (R³)₂N(O)C, R³O, (R³)₂N, R⁴(O)ₙS, R³O(O)₂S, (R³)₂N(O)₂S and R³O—(C₁-C₆)-alkyl, and where heterocyclyl carries n oxo groups,
R² represents (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₆)-cycloalkyl, (C₃-C₆)-cycloalkenyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, phenyl-N(R³)—(C₁-C₆)-alkyl, heteroaryl-N(R³)—(C₁-C₆)-alkyl, heterocyclyl-N(R³)—(C₁-C₆)-alkyl, phenyl-S(O)ₙ—(C₁-C₆)-alkyl, heteroaryl-S(O)ₙ—(C₁-C₆)-alkyl or heterocyclyl-S(O)ₙ—(C₁-C₆)-alkyl, where the fifteen last-mentioned radicals are in each case substituted by s radicals from the group consisting of nitro, halogen, cyano, thiocyanato, (C₁-C₆)-alkyl, halo-(C₁-C₆)-alkyl, (C₃-C₆)-cycloalkyl, R³O(O)C, (R³)₂N(O)C, R³O, (R³)₂N, R⁴(O)ₙS, R³O(O)₂S, (R³)₂N(O)₂S and R³O—(C₁-C₆)-alkyl, and where heterocyclyl carries n oxo groups,
R³ represents hydrogen, (C₁-C₆)-alkyl, halo-(C₁-C₆)-alkyl, (C₂-C₆)-alkenyl, (C₂-C₆)-alkynyl, (C₃-C₆)-cycloalkyl, (C₃-C₆)-cycloalkyl-(C₁-C₆)-alkyl or phenyl,
R⁴ is (C₁-C₆)-alkyl, halo-(C₁-C₆)-alkyl, (C₂-C₆)-alkenyl, (C₂-C₆)-alkynyl, (C₃-C₆)-cycloalkyl, (C₃-C₆)-cycloalkyl-(C₁-C₆)-alkyl or phenyl,
R⁵ represents hydrogen or (C₁-C₄)-alkyl,
R⁶ represents (C₁-C₄)-alkyl,
R′ represents acetoxy, acetamido, N-methylacetamido, benzoyloxy, benzamido, N-methylbenzamido, methoxycarbonyl, ethoxycarbonyl, benzoyl, methylcarbonyl, piperidinylcarbonyl, morpholinylcarbonyl, trifluoromethylcarbonyl, aminocarbonyl, methylaminocarbonyl, dimethylaminocarbonyl, (C₃-C₆)-cycloalkyl, or represents heteroaryl or heterocyclyl, in each case substituted by s radicals from the group consisting of methyl, ethyl, methoxy, trifluoromethyl and halogen,
n represents 0, 1 or 2,
s represents 0, 1, 2 or 3,
with the proviso that the compounds 4-difluoromethyl-3-ethylsulfinyl-2-methyl-N-(5-methyl-1,3,4-oxadiazol-2-yl)benzamide and 4-difluoromethyl-3-ethylsulfonyl-2-methyl-N-(5-methyl-1,3,4-oxadiazol-2-yl)benzamide and sodium salts thereof are excluded.

2. The benzoylamide as claimed in claim 1, in which
Q represents a radical Q1, Q2, Q3 or Q4, 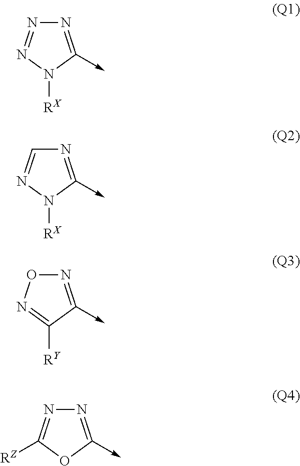
X represents (C₁-C₆)-alkyl or (C₃-C₆)-cycloalkyl,
R represents (C₁-C₆)-alkyl, (C₃-C₆)-cycloalkyl, (C₃-C₆)-cycloalkyl-(C₁-C₆)-alkyl, (C₁-C₆)-alkyl (C₁-C₆)-alkyl,
R^{a} represents hydrogen,
R^{X} represents (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, where the six radicals mentioned above are in each case substituted by s radicals from the group consisting of R²(O)ₙS, (R¹)₂N, R¹O, R¹(O)C, R¹O(O)C, R¹(O)CO, R²O(O)CO, R¹(O)C(R¹)N, R²(O)₂S(R¹)N, (C₃-C₆)-cycloalkyl, heteroaryl, heterocyclyl and phenyl, where the four last-mentioned radicals are substituted by s radicals from the group consisting of (C₁-C₆)-alkyl, halo-(C₁-C₆)-alkyl, (C₁-C₆)-alkoxy and halogen, and where heterocyclyl carries n oxo groups,
or R^{X} represents (C₃-C₇)-cycloalkyl, where this radical is substituted by s radicals from the group consisting of halogen, (C₁-C₆)-alkyl and halo-(C₁-C₆)-alkyl,
R^{Y} represents hydrogen, (C₁-C₆)-alkyl, halo-(C₁-C₆)-alkyl, (C₃-C₇)-cycloalkyl, (C₁-C₆)-alkoxy, methoxycarbonyl, methoxycarbonylmethyl, halogen, amino, aminocarbonyl or methoxymethyl,
R^{Z} represents hydrogen, (C₁-C₆)-alkyl, R¹O—(C₁-C₆)-alkyl, R′CH₂, (C₃-C₇)-cycloalkyl, halo-(C₁-C₆)-alkyl, R¹O, R¹(H)N, methoxycarbonyl, acetylamino or methylsulfonyl,
R¹ represents hydrogen, (C₁-C₆)-alkyl, halo-(C₁-C₆)-alkyl, (C₃-C₆)-cycloalkyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, where the nine last-mentioned radicals are in each case substituted by s radicals from the group consisting of nitro, halogen, (C₁-C₆)-alkyl, halo-(C₁-C₆)-alkyl, R³O(O)C, (R³)₂N(O)C, R³O, (R³)₂N, R⁴(O)ₙS and R³O—(C₁-C₆)-alkyl, and where heterocyclyl carries n oxo groups,
R² represents (C₁-C₆)-alkyl, halo-(C₁-C₆)-alkyl, (C₃-C₆)-cycloalkyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, where the nine last-mentioned radicals are in each case substituted by s radicals from the group consisting of nitro, halogen, (C₁-C₆)-alkyl, halo-(C₁-C₆)-alkyl, R³O(O)C, (R³)₂N(O)C, R³O, (R³)₂N, R⁴(O)ₙS and R³O—(C₁-C₆)-alkyl, and where heterocyclyl carries n oxo groups,
R³ represents hydrogen or (C₁-C₆)-alkyl,
R⁴ represents (C₁-C₆)-alkyl,
R′ represents acetoxy, acetamido, methoxycarbonyl or (C₃-C₆)-cycloalkyl,
n represents 0, 1 or 2,
s represents 0, 1, 2 or 3.

3. The benzoylamide as claimed in claim 1, in which
Q represents a radical Q1, Q2, Q3 or Q4, 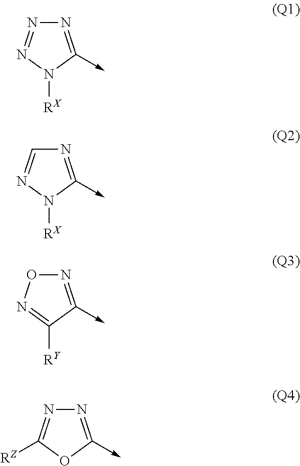
X represents halogen,
R represents (C₁-C₆)-alkyl, (C₃-C₆)-cycloalkyl, (C₃-C₆)-cycloalkyl-(C₁-C₆)-alkyl, (C₁-C₆)-alkyl-O—(C₁-C₆)-alkyl,
R^{a} represents hydrogen,
R^{X} represents (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, where the six radicals mentioned above are in each case substituted by s radicals from the group consisting of R²(O)ₙS, (R¹)₂N, R¹O, R¹(O)C, R¹O(O)C, R¹(O)CO, R²O(O)CO, R¹(O)C(R¹)N, R²(O)₂S(R¹)N, (C₃-C₆)-cycloalkyl, heteroaryl, heterocyclyl and phenyl, where the four last-mentioned radicals are substituted by s radicals from the group consisting of (C₁-C₆)-alkyl, halo-(C₁-C₆)-alkyl, (C₁-C₆)-alkoxy and halogen, and where heterocyclyl carries n oxo groups,
or R^{X} represents (C₃-C₇)-cycloalkyl, where this radical is substituted by s radicals from the group consisting of halogen, (C₁-C₆)-alkyl and halo-(C₁-C₆)-alkyl,
R^{Y} represents hydrogen, (C₁-C₆)-alkyl, halo-(C₁-C₆)-alkyl, (C₃-C₇)-cycloalkyl, (C₁-C₆)-alkoxy, methoxycarbonyl, methoxycarbonylmethyl, halogen, amino, aminocarbonyl or methoxymethyl,
R^{Z} represents hydrogen, (C₁-C₆)-alkyl, R¹O—(C₁-C₆)-alkyl, R′CH₂, (C₃-C₇)-cycloalkyl, halo-(C₁-C₆)-alkyl, R¹O, R¹(H)N, methoxycarbonyl, acetylamino or methylsulfonyl,
R¹ represents hydrogen, (C₁-C₆)-alkyl, halo-(C₁-C₆)-alkyl, (C₃-C₆)-cycloalkyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, where the nine last-mentioned radicals are in each case substituted by s radicals from the group consisting of nitro, halogen, (C₁-C₆)-alkyl, halo-(C₁-C₆)-alkyl, R³O(O)C, (R³)₂N(O)C, R³O, (R³)₂N, R⁴(O)ₙS and R³O—(C₁-C₆)-alkyl, and where heterocyclyl carries n oxo groups,
R² represents (C₁-C₆)-alkyl, halo-(C₁-C₆)-alkyl, (C₃-C₆)-cycloalkyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, where the nine last-mentioned radicals are in each case substituted by s radicals from the group consisting of nitro, halogen, (C₁-C₆)-alkyl, halo-(C₁-C₆)-alkyl, R³O(O)C, (R³)₂N(O)C, R³O, (R³)₂N, R⁴(O)ₙS and R³O—(C₁-C₆)-alkyl, and where heterocyclyl carries n oxo groups,
R³ represents hydrogen or (C₁-C₆)-alkyl,
R⁴ represents (C₁-C₆)-alkyl,
R′ represents acetoxy, acetamido, methoxycarbonyl or (C₃-C₆)-cycloalkyl,
n represents 0, 1 or 2,
s represents 0, 1, 2 or 3.

4. The benzoylamide as claimed in claim 1, in which
Q represents a radical Q1, Q2, Q3 or Q4, 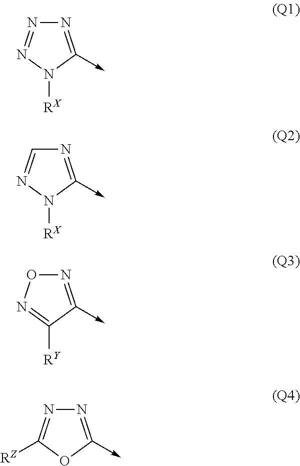
X represents methyl, ethyl or cyclopropyl,
R represents methyl, ethyl, cyclopropylmethyl or methoxyethyl,
R^{a} represents hydrogen,
R^{X} represents methyl, ethyl or n-propyl,
R^{Y} represents methyl or chlorine,
R^{Z} represents methyl,
n represents 0, 1 or 2.

5. The benzoylamide as claimed in claim 1, in which
Q represents a radical Q1, Q2, Q3 or Q4, 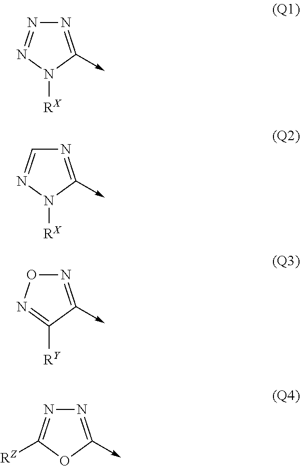
X represents fluorine, chlorine, bromine or iodine,
R represents methyl, ethyl, cyclopropylmethyl or methoxyethyl,
R^{a} represents hydrogen,
R^{X} represents methyl, ethyl or n-propyl,
R^{Y} represents methyl or chlorine,
R^{Z} represents methyl,
n represents 0, 1 or 2.

6. A herbicidal composition comprising at least one compound as claimed in claim 1 mixed with one or more formulation auxiliaries.

7. The herbicidal composition as claimed in claim 6, comprising at least one further pesticidally active substance selected from the group consisting of insecticides, acaricides, herbicides, fungicides, safeners, and growth regulators.

8. A method for controlling one or more unwanted plants, comprising applying an effective amount of at least one compound of the formula (I) as claimed in claim 1 or of herbicidal compositions thereof is applied to the plants or a site of unwanted vegetation.

9. A product comprising a compound as claimed in claim 1 or of herbicidal compositions thereof for controlling one or more unwanted plants.

10. The product as claimed in claim 9, wherein the compound is used for controlling unwanted plants in one or more crops of useful plants.

11. The product as claimed in claim 10, wherein the useful plants are transgenic useful plants.

12. A compound of the formula (II) 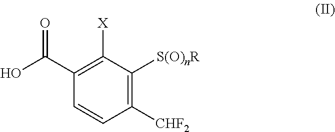
in which the symbols and indices are defined as follows:
X represents (C₃-C₆)-cycloalkyl or halogen,
R represents (C₁-C₆)-alkyl, (C₃-C₆)-cycloalkyl, (C₃-C₆)-cycloalkyl-(C₁-C₆)-alkyl, (C₁-C₆)-alkyl (C₁-C₆)-alkyl,
n represents 0, 1 or 2.

13. The compound as claimed in claim 12, in which
X represents cyclopropyl, fluorine, chlorine, bromine or iodine,
R represents methyl, ethyl, cyclopropylmethyl or methoxyethyl,
n represents 0, 1 or 2.

14. A compound of the formula (III), 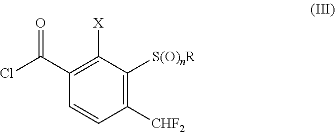
in which the symbols and indices are defined as follows:
X represents (C₁-C₆)-alkyl, (C₃-C₆)-cycloalkyl or halogen,
R represents (C₁-C₆)-alkyl, (C₃-C₆)-cycloalkyl, (C₃-C₆)-cycloalkyl-(C₁-C₆)-alkyl, (C₁-C₆)-alkyl-O—(C₁-C₆)-alkyl,
n represents 0, 1 or 2.

15. The compound as claimed in claim 14, in which
X represents methyl, ethyl, cyclopropyl, fluorine, chlorine, bromine or iodine,
R represents methyl, ethyl, cyclopropylmethyl or methoxyethyl,
n represents 0, 1 or 2.
